(19) United States
(12) Patent Application Publication
(10) Pub. No.: US 2026/0124327 A1
(43) Pub. Date: May 7, 2026

(54) Compounds Targeting Carbonic Anhydrase IX and Uses Thereof

(71) Applicant: RayzeBio, Inc., San Diego, CA (US)

(72) Inventors: Andrew R. Hudson, San Diego, CA (US); Nicholas D. Smith, San Diego, CA (US); Joe A. Tran, San Marcos, CA (US); Yalda Bravo, San Diego, CA (US)

(73) Assignee: RayzeBio, Inc., San Diego, CA (US)

(21) Appl. No.: 19/379,951

(22) Filed: Nov. 5, 2025

Related U.S. Application Data
(60) Provisional application No. 63/716,788, filed on Nov. 6, 2024.

(51) Int. Cl. A61K 51/06; A61K 51/04; C07B 59/00; C07D 417/14; C07D 513/04; C07F 1/08; C07F 5/00; C07F 7/24

(52) U.S. Cl. CPC A61K 51/065; A61K 51/0459; A61K 51/0482; C07B 59/004; C07D 417/14; C07D 513/04; C07F 1/08; C07F 5/003; C07F 7/24; C07B 2200/05

(57) ABSTRACT

Disclosed herein are compounds that target carbonic anhydrase 9 (CA IX) (such as compounds of structural formula (I) or (II) wherein values for variables are as described herein) and methods for the prevention and/or treatment of certain cancers, including solid tumors, using such compounds. Also disclosed herein are pharmaceutically acceptable salts of such compounds and pharmaceutical compositions comprising such compounds or salts for use in such methods of preventing or treating cancers.

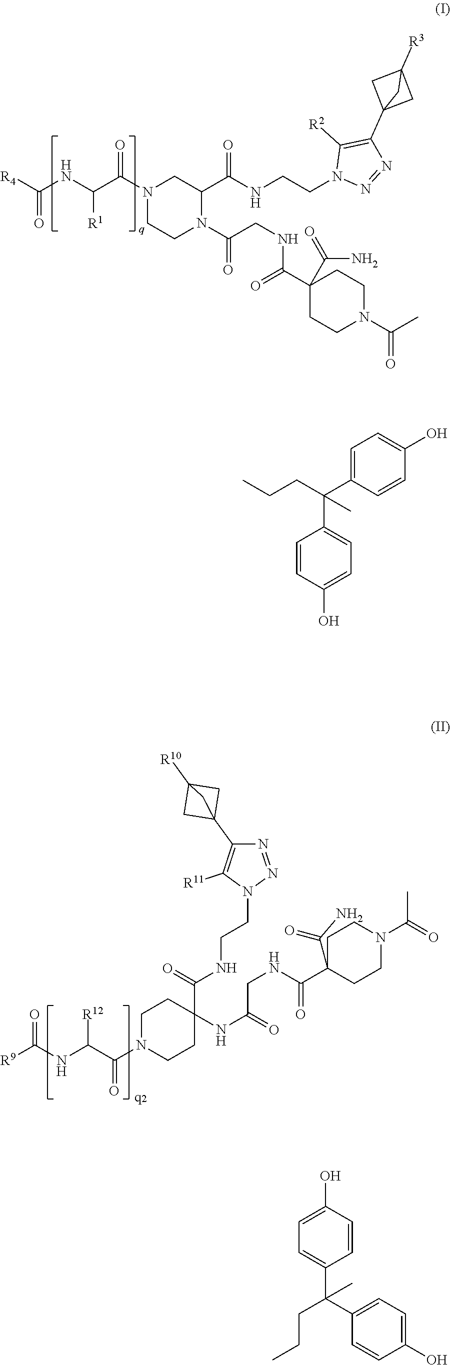

### CROSS-REFERENCE TO RELATED APPLICATIONS

This application claims the benefit of priority of U.S. Provisional Application No. 63/716,788, filed on Nov. 6, 2024, which is incorporated herein by reference in its entirety for any purpose.

### FIELD

The present disclosure relates generally to compounds, compositions, and methods for their preparation, and use of the compounds and compositions for treating diseases and conditions associated with the expression of carbonic anhydrase 9.

### BACKGROUND

The expression of distinct proteins on the surface of tumor cells offers the opportunity to diagnose and characterize disease by probing the phenotypic identity and biochemical composition and activity of the tumor. Radioactive molecules that selectively bind to specific tumor cell surface proteins allow the use of noninvasive imaging techniques, such as molecular imaging or nuclear medicine, for detecting the presence and quantity of tumor associated proteins, thereby providing vital information related to the diagnosis and extent of disease, prognosis, and therapeutic management options. As radiopharmaceuticals can be prepared that are not only capable of imaging disease but also delivering a therapeutic radionuclide to the diseased tissue, therapy, in particular cancer therapy, can be realized. The selective expression of carbonic anhydrase 9 (CA-IX, or CA9) on tumors in response to hypoxia makes it an attractive target to exploit for noninvasive imaging as well as targeted radiotherapy.

In order to grow beyond more than a few millimeters in diameter, tumor micrometastasis need to obtain a supply of oxygen to sustain the high metabolic rate characteristic of rapid growth, and do so by inducing the formation of new blood vessels. The distance that tumor cells reside from blood vessels is inversely proportional to the oxygen pressure of the tumor. Even when angiogenesis occurs and a blood supply is established, the less vascular interior region of the growing tumor mass remains hypoxic and eventually undergoes necrosis.

Hypoxia is associated with a poor response to radiation therapy, and leads to tumor resistance. Since oxygen is necessary for the cytotoxic actions of free radicals generated by radiation, higher, often incompatible, levels of radiation are required to promote damage to the tumor. Therefore, there is a need for non-invasive techniques to stratify patients based on cancer hypoxia who are not expected to respond to radiation therapy because of low oxygen, and who may be candidates for alternative hypoxia-activated chemotherapies that are becoming available. As hypoxia constitutes a major difference between the tumor and normal tissues, it can be exploited for the development of tumor specific probes.

Hypoxia is a potent stimulus for the expression of specific genes, several which function to trigger vasculogenesis and therefore supply oxygen to the tumor, increase metabolism to increase the oxygen extraction factor, and promote a favorable environment for tumor growth. The activation of hypoxia inducible genes is in part mediated by a transcription factor, HIF-1α. Under normoxic conditions, HIF-1α is hydroxylated on proline residues that reside in the oxygen induced degradation domain of the protein by proline hydroxylase. Hydroxyproline facilitates binding of Von-Hippel-Lindau Factor (VHL), a tumor suppressor that, when bound, promotes the ubiquitination and degradation of HIF-1α. During hypoxia, proline hydroxylase is inhibited, and VHL no longer binds HIF-1α; the now stabilized HIF-1α translocates to the nucleus and associates with HIF-1α. This heterodimeric transcription factor then binds to HIF-1 responsive DNA sequences in the promoter region of target genes including the carbonic anhydrase isoform CA-IX, as well as VEGF, erythropoietin, and glucose transporters.

Carbonic anhydrases are a family of enzymes comprised of 16 isozymes that catalyze the reaction: CO₂+H₂O [symbol] HCO₃⁻+H⁺, and therefore play an important role in pH regulation. Specific isozymes are found either in the cytosol, anchored to the membrane, within the mitochondria, or secreted from the cell. The well-studied constitutively expressed isozyme, carbonic anhydrase II, is found in the cytosol of most cell types, and is the primary isoform responsible for the regulation of intracellular pH.

CA-IX is a membrane-anchored isoform of the enzyme with its catalytic domain in the extracellular space. It has a limited tissue distribution and is found at low levels primarily in the gastrointestinal tract. The expression of CA-IX is under the control of HIF-1α, and this isozyme is highly expressed in tumor cells exposed to hypoxia both in vitro and in vivo. Increased CA-IX expression has been detected in carcinomas of the cervix, ovary, kidney, esophagus, lung, breast, and brain. CA-IX has been reported to promote extracellular acidification. The low extracellular pH as a result of the activity of CA-IX leads to tumorigenic transformation, chromosomal rearrangements, extracellular matrix breakdown, migration and invasion, induction of growth factors, protease activation, and chemoresistance.

Currently, many small molecule inhibitors for CA-IX show undesired side effects due to inhibition of other CA isozymes present in the target organ. There is a need for a CA-IX selective inhibitor to potentially reduce undesired side effects that may arise from inhibition of other, including non-membrane-associated, CA isozymes.

### SUMMARY

Disclosed herein are compounds targeting the CA-IX receptor (e.g., compounds that bind to the CA-IX receptor) and pharmaceutical compositions thereof. In some embodiments, the compounds disclosed herein are selective for CA-IX over CA-XII. Certain compounds described herein bind to CA IX, e.g., with strong affinity, and demonstrate reduced stomach uptake when administered to a subject.

In some embodiments, the compounds disclosed herein also bind to albumin, e.g., with high affinity. In some embodiments, compounds disclosed herein that bind to albumin have reduced uptake in the stomach.

The compounds can be useful for treating cancer. The compounds can also be useful in imaging and disease diagnosis.

In certain embodiments, the compound is of structural formula (I) or (II):

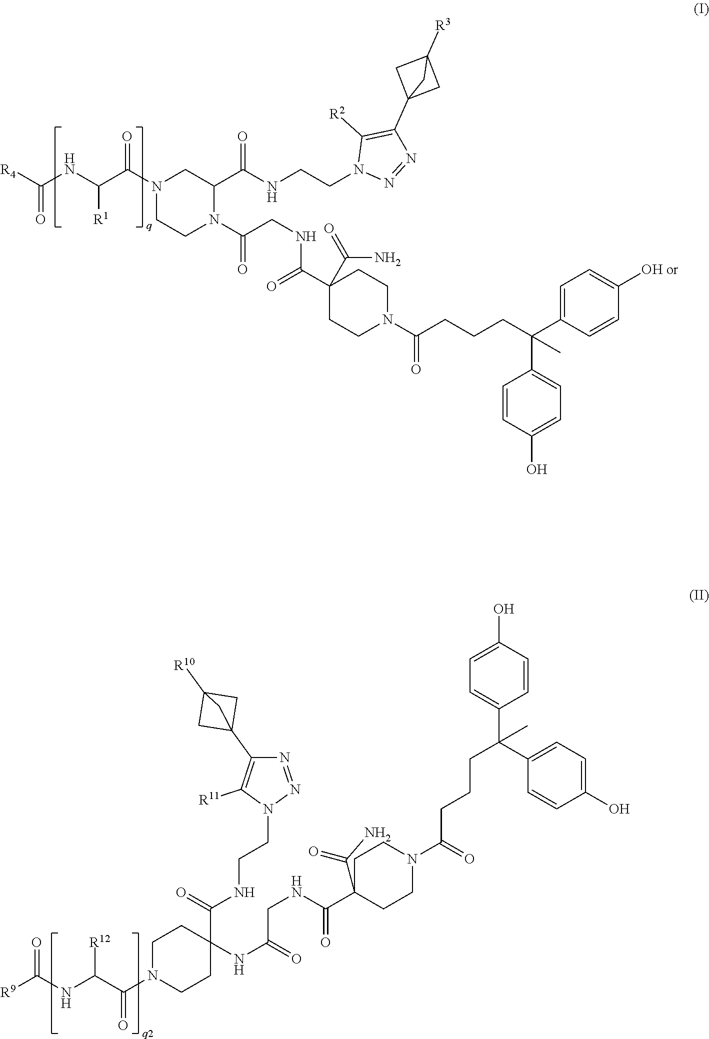

or a stereoisomer or mixture of stereoisomers, or pharmaceutically acceptable salt thereof, wherein values for the variables (e.g., R¹, R², R³, R₄, R⁹, R¹⁰, R¹¹, R¹², q, q2) are as described herein.

Also disclosed herein are pharmaceutical compositions comprising a compound of structural formula (I) or (II) (e.g., a radiopharmaceutical compound of structural formula (I) or (II)), or a stereoisomer or mixture of stereoisomers, or pharmaceutically acceptable salt thereof, and a pharmaceutically acceptable excipient or carrier.

Also disclosed herein is a method of diagnosing or imaging a disease or disorder in a subject in need thereof, the method comprising administering to the subject an effective amount of a compound of structural formula (I) or (II) (e.g., a radiopharmaceutical compound of structural formula (I) or (II)), or a stereoisomer or mixture of stereoisomers, or pharmaceutically acceptable salt thereof, or a pharmaceutical composition comprising a compound of structural formula (I) or (II) (e.g., a radiopharmaceutical compound of structural formula (I) or (II)), or a stereoisomer or mixture of stereoisomers, or pharmaceutically acceptable salt thereof.

Also disclosed herein is a method of treating a disease or disorder in a subject in need thereof, the method comprising administering to the subject a therapeutically effective amount of a compound of structural formula (I) or (II) (e.g., a radiopharmaceutical compound of structural formula (I) or (II)), or a stereoisomer or mixture of stereoisomers, or pharmaceutically acceptable salt thereof, or a pharmaceutical composition comprising a compound of structural formula (I) or (II) (e.g., a radiopharmaceutical compound of structural formula (I) or (II)), or a stereoisomer or mixture of stereoisomers, or pharmaceutically acceptable salt thereof.

Also disclosed herein is a composition for a use described herein (e.g., diagnosing and/or imaging a disease or disorder described herein; treating a disease or disorder described herein), wherein the composition is a composition disclosed herein. Also disclosed herein is use of a compound (e.g., a compound of structural formula (I) or (II), or a stereoisomer or mixture of stereoisomers, or pharmaceutically acceptable salt thereof) or composition disclosed herein for, e.g., diagnosing and/or imaging a disease or disorder described herein and/or treating a disease or disorder described herein. Also disclosed herein is use of a compound (e.g., a compound of structural formula (I) or (II), or a stereoisomer or mixture of stereoisomers, or pharmaceutically acceptable salt thereof) or composition disclosed herein for the manufacture of a medicament for a use described herein (e.g., diagnosing and/or imaging a disease or disorder described herein; treating a disease or disorder described herein).

The present embodiments can be understood more fully by reference to the detailed description and examples, which are intended to exemplify non-limiting embodiments.

Further non-limiting, numbered embodiments include:

Embodiment 1. A compound of the following structural formula:

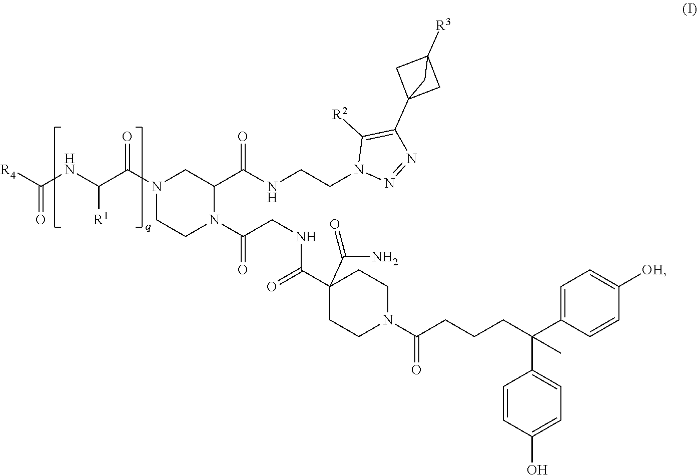

or a pharmaceutically acceptable salt thereof, wherein:
q is 0 or 1;
R¹ is

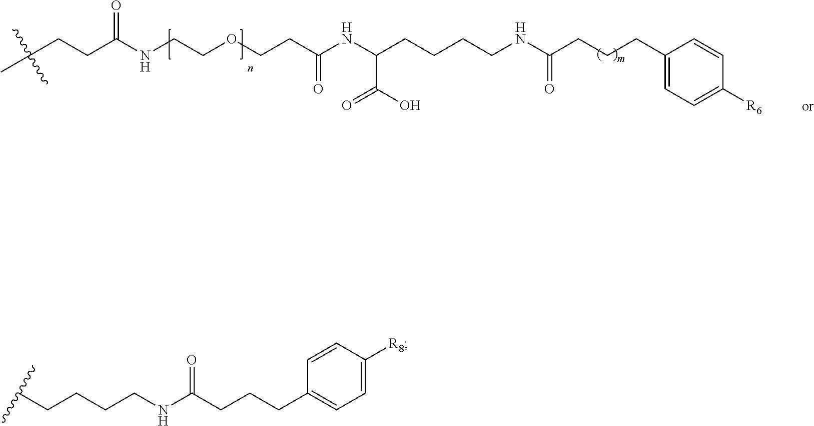

n is 2, 3, 4, 5, 6, 7, 8, 9, 10, 11, 12, 13, 14, 15, or 16;
m is 1, 2, or 3;
   R⁶ is H, halogen, —CH₃, —OCH₃, or —OCH₂CH₃;
R₈ is halogen or —CH₃;
R² is H or —COOH;
R³ is

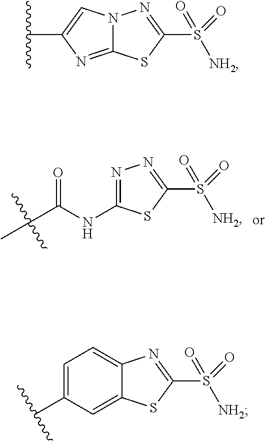 [SMILES: *c1cn2nc(S(N)(=O)=O)sc2n1 ; *C(=O)Nc1nnc(S(N)(=O)=O)s1 ; "or" ; *c1ccc2nc(S(N)(=O)=O)sc2c1]

and
R⁴ comprises a metal chelator (CL).

Embodiment 2. The compound of embodiment 1, wherein q is 0.

Embodiment 3. The compound of embodiment 1, wherein q is 1.

Embodiment 4. The compound of embodiment 1, of the following structural formula:

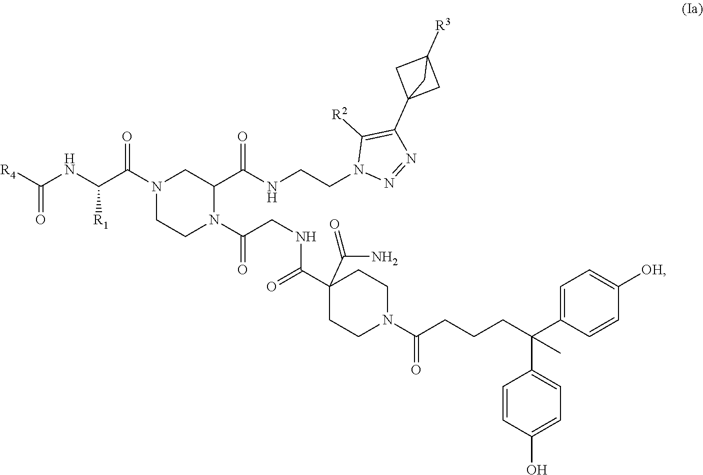

or a pharmaceutically acceptable salt thereof.

Embodiment 5. The compound of any one of embodiments 1-4, where R³ is

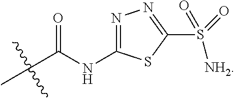 [SMILES: *C(=O)Nc1nnc(S(N)(=O)=O)s1]

Embodiment 6. The compound of any one of embodiments 1-4, where R³ is

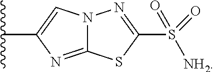 [SMILES: *c1cn2nc(S(N)(=O)=O)sc2n1]

Embodiment 7. The compound of any one of embodiments 1-4, where R³ is

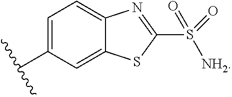 [SMILES: *c1ccc2nc(S(N)(=O)=O)sc2c1]

Embodiment 8. The compound of any one of embodiments 1-7, wherein the metal chelator (CL) is NOTA, DOTA, or DOTA-monoamide.

Embodiment 9. The compound of any one of embodiments 1-8, wherein R⁴ is

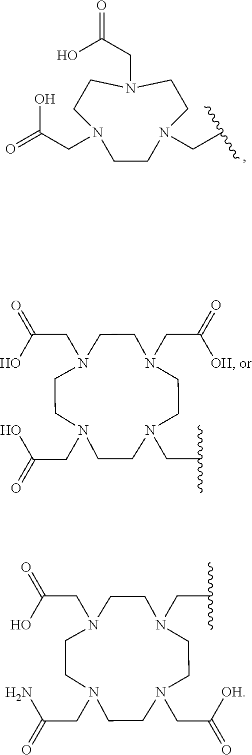

Embodiment 10. The compound of embodiment 9, wherein R⁴ is

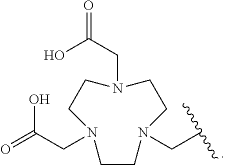 [SMILES: *CN1CCN(CC(=O)O)CCN(CC(=O)O)CC1]

Embodiment 11. The compound of embodiment 9, wherein R⁴ is

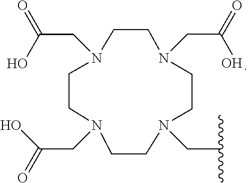 [SMILES: *CN1CCN(CC(=O)O)CCN(CC(=O)O)CCN(CC(=O)O)CC1]

Embodiment 12. The compound of embodiment 9, wherein R⁴ is

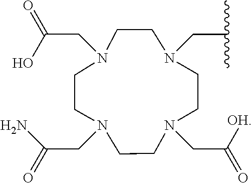 [SMILES: *CN1CCN(CC(=O)O)CCN(CC(N)=O)CCN(CC(=O)O)CC1]

Embodiment 13. The compound of any one of embodiments 1-12, wherein R² is H.

Embodiment 14. The compound of any one of embodiments 1-12, wherein R² is —COOH.

Embodiment 15. The compound of any one of embodiments 1-14, wherein R¹ is

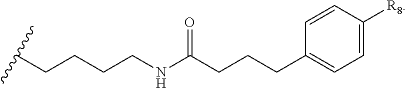

Embodiment 16. The compound of any one of embodiments 1-15, wherein R₈ is —CH₃.

Embodiment 17. The compound of any one of embodiments 1-15, wherein R₈ is halogen.

Embodiment 18. The compound of embodiment 17, wherein R₈ is F.

Embodiment 19. The compound of embodiment 17, wherein R₈ is Cl.

Embodiment 20. The compound of embodiment 17, wherein R₈ is I.

Embodiment 21. The compound of embodiment 17, wherein R₈ is Br.

Embodiment 22. The compound of any one of embodiments 1-14, wherein R¹ is

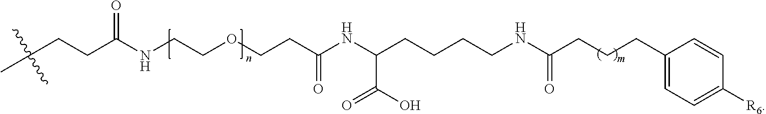

Embodiment 23. The compound of any one of embodiments 1-15, wherein R¹ is

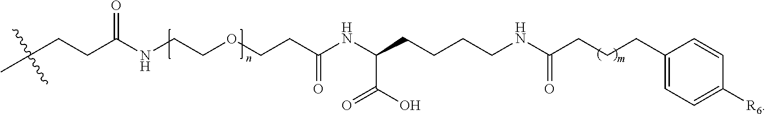

Embodiment 24. The compound of any one of embodiments 1-14, 22, and 23, wherein n is 6, 8, or 16.

Embodiment 25. The compound of embodiment 24, wherein n is 6.

Embodiment 26. The compound of embodiment 24, wherein n is 8.

Embodiment 27. The compound of embodiment 24, wherein n is 16.

Embodiment 28. The compound of any one of embodiments 1-14 and 22-27, wherein mis 1.

Embodiment 29. The compound of any one of embodiments 1-14 and 22-27, wherein m is 2.

Embodiment 30. The compound of any one of embodiments 1-14 and 22-27, wherein m is 3.

Embodiment 31. The compound of any one of embodiments 1-14 and 22-30, wherein R⁶ is H.

Embodiment 32. The compound of any one of embodiments 1-14 and 22-30, wherein R⁶ is a halogen.

Embodiment 33. The compound of embodiment 32, wherein R⁶ is F.

Embodiment 34. The compound of embodiment 32, wherein R⁶ is Cl.

Embodiment 35. The compound of embodiment 32, wherein R⁶ is Br.

Embodiment 36. The compound of embodiment 32, wherein R⁶ is I.

Embodiment 37. The compound of any one of embodiments 1-14 and 22-30, wherein R⁶ is —CH₃.

Embodiment 38. The compound of any one of embodiments 1-14 and 22-30, wherein R⁶ is —OCH₃.

Embodiment 39. The compound of any one of embodiments 1-14 and 22-30, wherein R⁶ is —OCH₂CH₃.

Embodiment 40. The compound of any one of embodiments 1-39, further comprising a radionuclide bound to the metal chelator (CL).

Embodiment 41. The compound of embodiment 40, wherein the radionuclide is an alpha particle-emitting radionuclide.

Embodiment 42. The compound of embodiment 41, wherein the alpha particle-emitting radionuclide is Ac-225, Bi-213, Bi-209, Tb-149, Ra-223, Th-227, Fr-223, Gd-148, Th-229, Pb-212, or Po-213.

Embodiment 43. The compound of embodiment 42, wherein the alpha particle-emitting radionuclide is Ac-225.

Embodiment 44. The compound of embodiment 40, wherein the radionuclide is a beta particle-emitting radionuclide.

Embodiment 45. The compound of embodiment 44, wherein the beta particle-emitting radionuclide is Cu-67, Lu-177, Y-90, Rh-105, Yb-175, Tm-167, Pm-153, Sm-153, or In-111.

Embodiment 46. The compound of embodiment 45, wherein the beta particle-emitting radionuclide is lutetium-177.

Embodiment 47. The compound of embodiment 40, wherein the radionuclide is a positron-emitting radionuclide.

Embodiment 48. A compound having a structure in Table 2, or a pharmaceutically acceptable salt thereof.

Embodiment 49. A compound of the following structural formula:

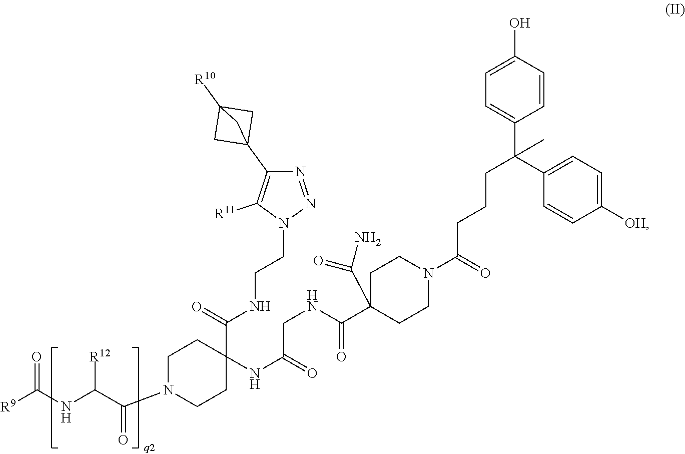

or a pharmaceutically acceptable salt thereof, wherein:
q2 is 0 or 1;

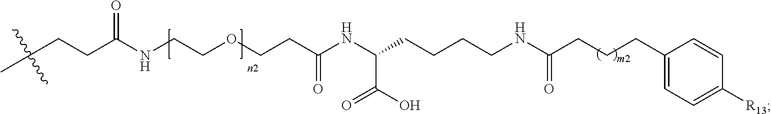

R¹² is
   n2 is 2, 3, 4, 5, 6, 7, 8, 9, 10, 11, 12, 13, 14, 15, or 16;
m2 is 1, 2, or 3;
   R₁₃ is H, halogen, —CH₃, —OCH₃, or —OCH₂CH₃;
R¹¹ is H or —COOH;
R¹⁰ is

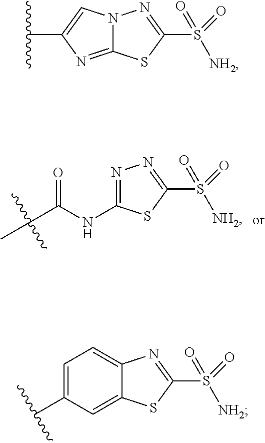 [SMILES: *c1cn2nc(S(N)(=O)=O)sc2n1 ; *C(=O)Nc1nnc(S(N)(=O)=O)s1 ; "or" ; *c1ccc2nc(S(N)(=O)=O)sc2c1]

and
R⁹ comprises a metal chelator (CL).

Embodiment 50. The compound of embodiment 49, wherein q2 is 0.

Embodiment 51. The compound of embodiment 49, wherein q2 is 1.

Embodiment 52. The compound of embodiment 49, of the following structural formula:

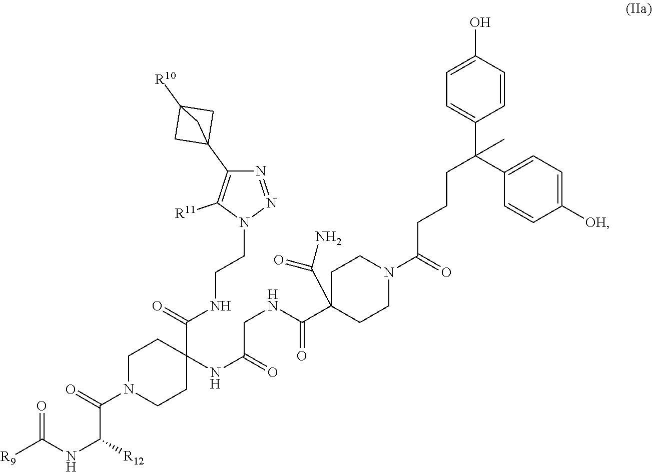

or a pharmaceutically acceptable salt thereof.

Embodiment 53. The compound of any one of embodiments 49-52, where R¹⁰ is

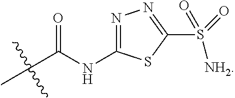 [SMILES: *C(=O)Nc1nnc(S(N)(=O)=O)s1]

Embodiment 54. The compound of any one of embodiments 49-52, where R¹⁰ is

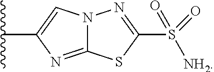 [SMILES: *c1cn2nc(S(N)(=O)=O)sc2n1]

Embodiment 55. The compound of any one of embodiments 49-52, where R¹⁰ is

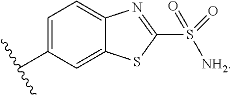 [SMILES: *c1ccc2nc(S(N)(=O)=O)sc2c1]

Embodiment 56. The compound of any one of embodiments 49-55, wherein the metal chelator (CL) is NOTA, DOTA, or DOTA-monoamide.

Embodiment 57. The compound of any one of embodiments 49-56, wherein R⁹ is

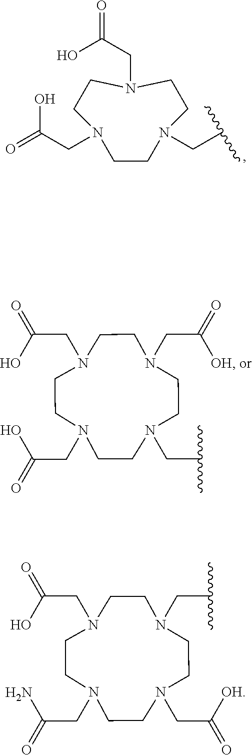

Embodiment 58. The compound of embodiment 57, wherein R⁹ is

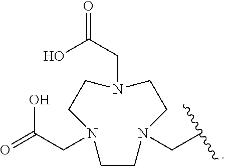 [SMILES: *CN1CCN(CC(=O)O)CCN(CC(=O)O)CC1]

Embodiment 59. The compound of embodiment 57, wherein R⁹ is

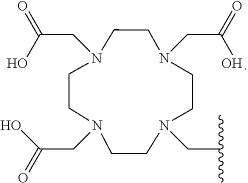 [SMILES: *CN1CCN(CC(=O)O)CCN(CC(=O)O)CCN(CC(=O)O)CC1]

Embodiment 60. The compound of embodiment 57, wherein R⁹ is

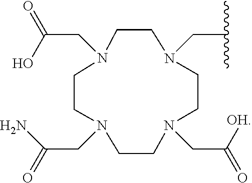 [SMILES: *CN1CCN(CC(=O)O)CCN(CC(N)=O)CCN(CC(=O)O)CC1]

Embodiment 61. The compound of any one of embodiments 49-59, wherein R₁₁ is H.

Embodiment 62. The compound of any one of embodiments 49-59, wherein R₁₁ is —COOH.

Embodiment 63. The compound of any one of embodiments 49-62, wherein R¹² is

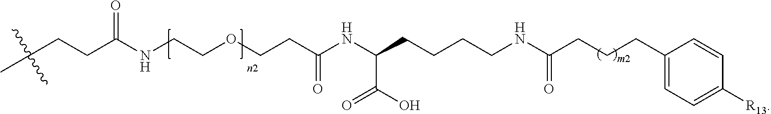

Embodiment 64. The compound of any one of embodiments 49-63, wherein n2 is 6, 8, or 16.

Embodiment 65. The compound of embodiment 64, wherein n2 is 6.

Embodiment 66. The compound of embodiment 64, wherein n2 is 8.

Embodiment 67. The compound of embodiment 64, wherein n2 is 16.

Embodiment 68. The compound of any one of embodiments 49-67, wherein m2 is 1.

Embodiment 69. The compound of any one of embodiments 49-67, wherein m2 is 2.

Embodiment 70. The compound of any one of embodiments 49-67, wherein m2 is 3.

Embodiment 71. The compound of any one of embodiments 49-70, wherein R¹³ is H.

Embodiment 72. The compound of any one of embodiments 49-70, wherein R¹³ is halogen.

Embodiment 73. The compound of embodiment 72, wherein R¹³ is F.

Embodiment 74. The compound of embodiment 72, wherein R¹³ is Cl.

Embodiment 75. The compound of embodiment 72, wherein R¹³ is Br.

Embodiment 76. The compound of embodiment 72, wherein R¹³ is I.

Embodiment 77. The compound of any one of embodiments 49-70, wherein R¹³ is —CH₃.

Embodiment 78. The compound of any one of embodiments 46-70, wherein R¹³ is —OCH₃.

Embodiment 79. The compound of any one of embodiments 49-70, wherein R¹³ is —OCH₂CH₃.

Embodiment 80. The compound of any one of embodiments 49-79, further comprising a radionuclide bound to the metal chelator (CL).

Embodiment 81. The compound of embodiment 80, wherein the radionuclide is an alpha particle-emitting radionuclide.

Embodiment 82. The compound of embodiment 81, wherein the alpha particle-emitting radionuclide is Ac-225, Bi-213, Bi-209, Tb-149, Ra-223, Th-227, Fr-223, Gd-148, Th-229, Pb-212, or Po-213.

Embodiment 83. The compound of embodiment 82, wherein the alpha particle-emitting radionuclide is Ac-225.

Embodiment 84. The compound of embodiment 80, wherein the radionuclide is a beta particle-emitting radionuclide.

Embodiment 85. The compound of embodiment 84, wherein the beta particle-emitting radionuclide is Cu-67, Lu-177, Y-90, Rh-105, Yb-175, Tm-167, Pm-153, Sm-153, or In-111.

Embodiment 86. The compound of embodiment 85, wherein the beta particle-emitting radionuclide is lutetium-177.

Embodiment 87. The radiopharmaceutical conjugate of embodiment 80, wherein the radionuclide is a positron-emitting radionuclide.

Embodiment 88. A compound having a structure in Table 1, or a pharmaceutically acceptable salt thereof.

Embodiment 89. A compound of the following structure:

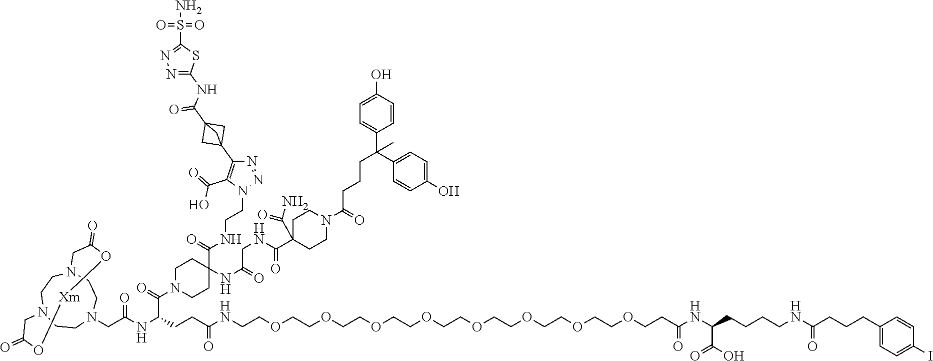

or a pharmaceutically acceptable salt thereof, wherein Xₘ is a radionuclide.

Embodiment 90. A compound of the following structure:

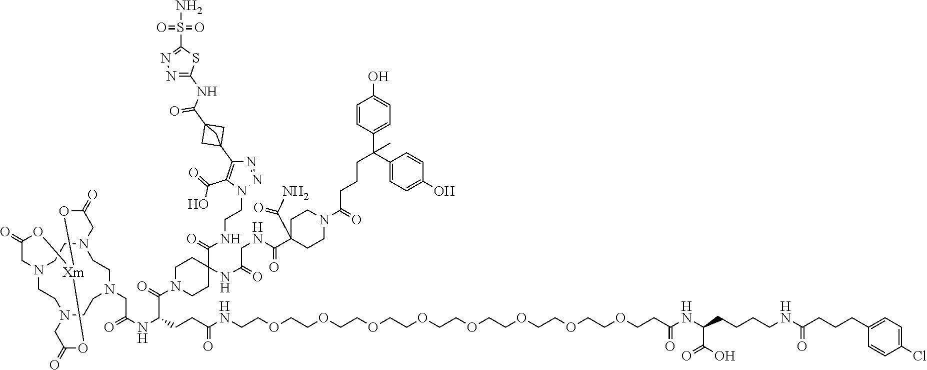

or a pharmaceutically acceptable salt thereof, wherein Xₘ is a radionuclide.

Embodiment 91. A compound of the following structure:

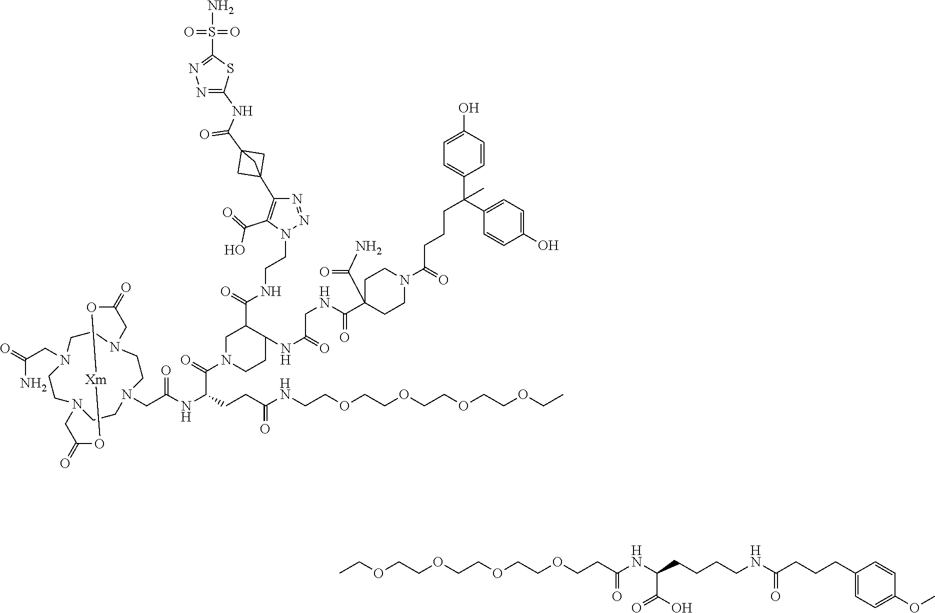

or a pharmaceutically acceptable salt thereof, wherein Xₘ is a radionuclide.

Embodiment 92. A compound of the following structure:

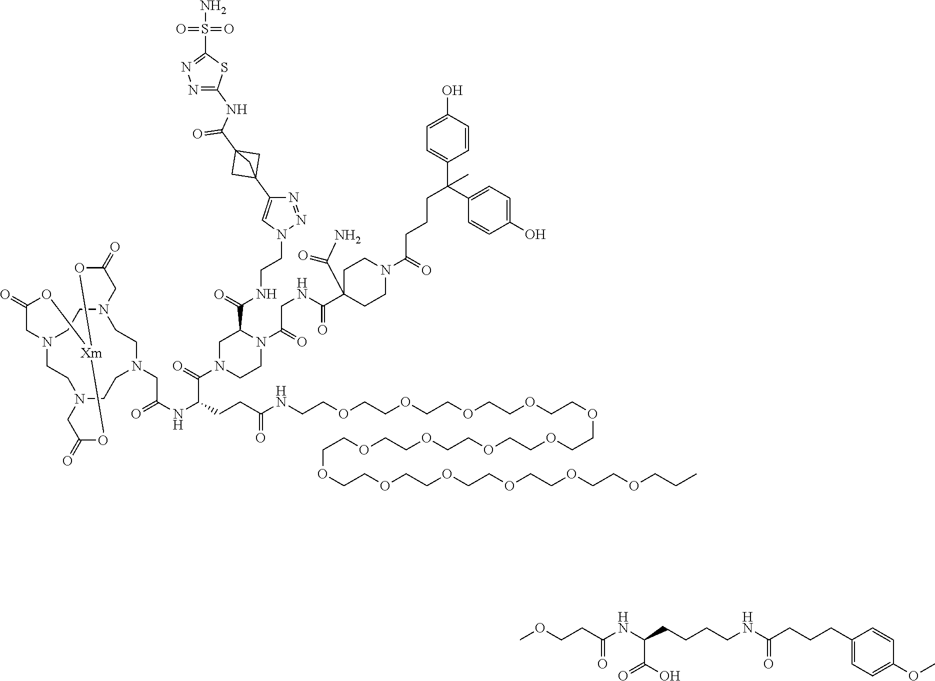

or a pharmaceutically acceptable salt thereof, wherein Xₘ is a radionuclide.

Embodiment 93. The compound of any one of embodiments 89-92, wherein the radionuclide is an alpha particle-emitting radionuclide.

Embodiment 94. The compound of embodiment 93, wherein the alpha particle-emitting radionuclide is Ac-225, Bi-213, Bi-209, Tb-149, Ra-223, Th-227, Fr-223, Gd-148, Th-229, Pb-212, or Po-213.

Embodiment 95. The compound of embodiment 94, wherein the alpha particle-emitting radionuclide is Ac-225.

Embodiment 96. The compound of any one of embodiments 89-92, wherein the radionuclide is a beta particle-emitting radionuclide.

Embodiment 97. The compound of embodiment 96, wherein the beta particle-emitting radionuclide is Cu-67, Lu-177, Y-90, Rh-105, Yb-175, Tm-167, Pm-153, Sm-153, or In-111.

Embodiment 98. The compound of embodiment 97, wherein the beta particle-emitting radionuclide is Lu-177.

Embodiment 99. The compound of any one of embodiments 40, 80, and 89-92, wherein the radionuclide is Cu-67, Lu-177, Ac-225, or Ga-68.

Embodiment 100. A pharmaceutical composition comprising a compound of any one of embodiments 1-99, or a pharmaceutically acceptable salt thereof, and a pharmaceutically acceptable excipient or carrier.

Embodiment 101. A method of diagnosing or imaging a disease or disorder in a subject in need thereof, the method comprising administering to the subject an effective amount of a compound of any one of embodiments 40-48 and 80-99, or a pharmaceutically acceptable salt thereof, or the pharmaceutical composition of embodiment 100.

Embodiment 102. A method of treating a disease or disorder in a subject in need thereof, the method comprising administering to the subject a therapeutically effective amount of the compound of any one of embodiments 40-48 and 80-99, or a pharmaceutically acceptable salt thereof, or the pharmaceutical composition of embodiment 100.

Embodiment 103. The method of embodiment 101 or 102, wherein the disease or disorder is a cancer.

Embodiment 104. The method of embodiment 103, wherein the cancer is hypoxic.

Embodiment 105. The method of embodiment 103 or 104, wherein the cancer is renal cell carcinoma.

Embodiment 106. The method of any one of embodiments 103-105, wherein the cancer is clear cell renal cell carcinoma.

### BRIEF DESCRIPTION OF THE DRAWINGS

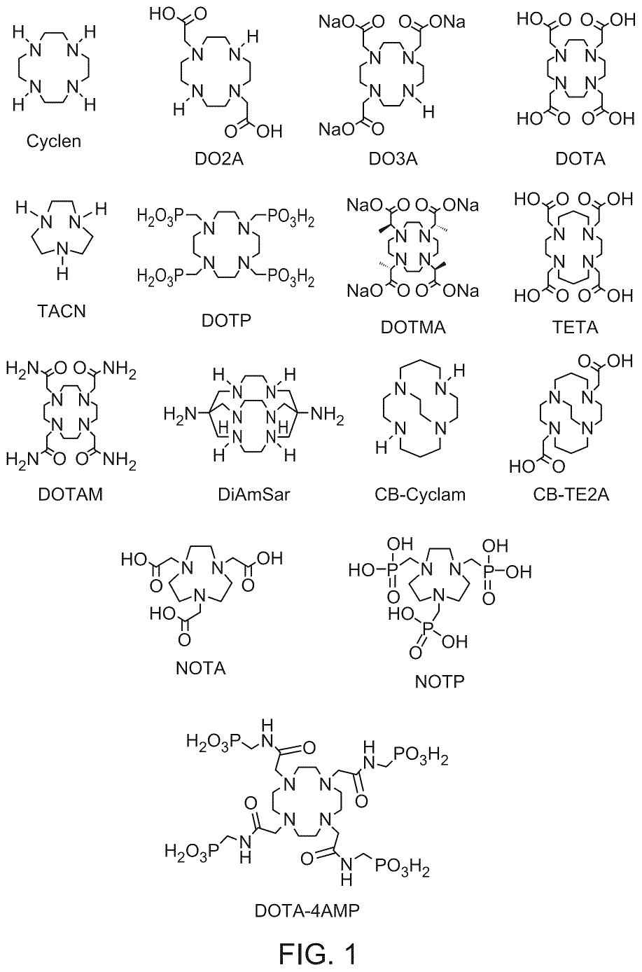

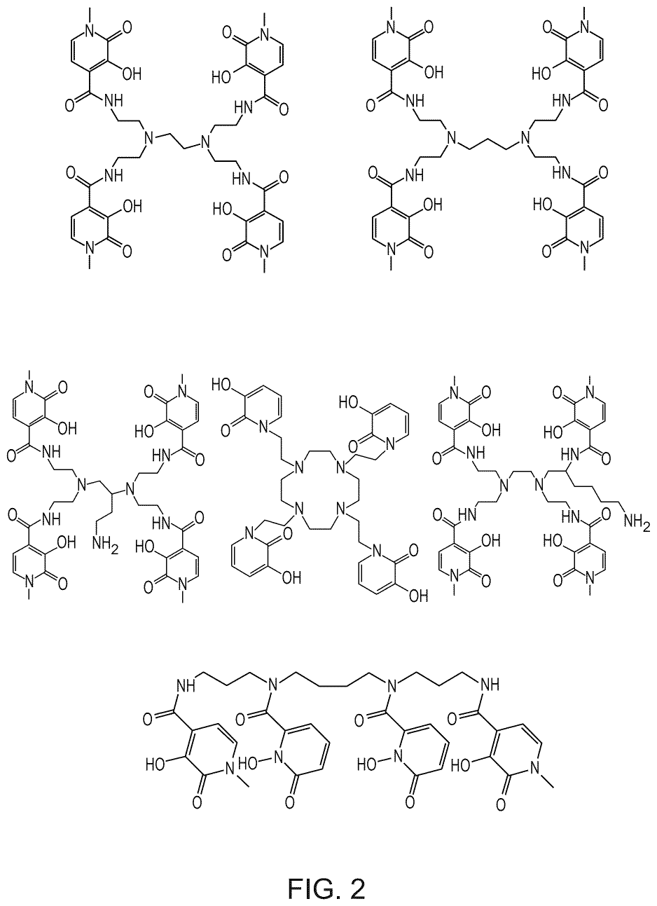

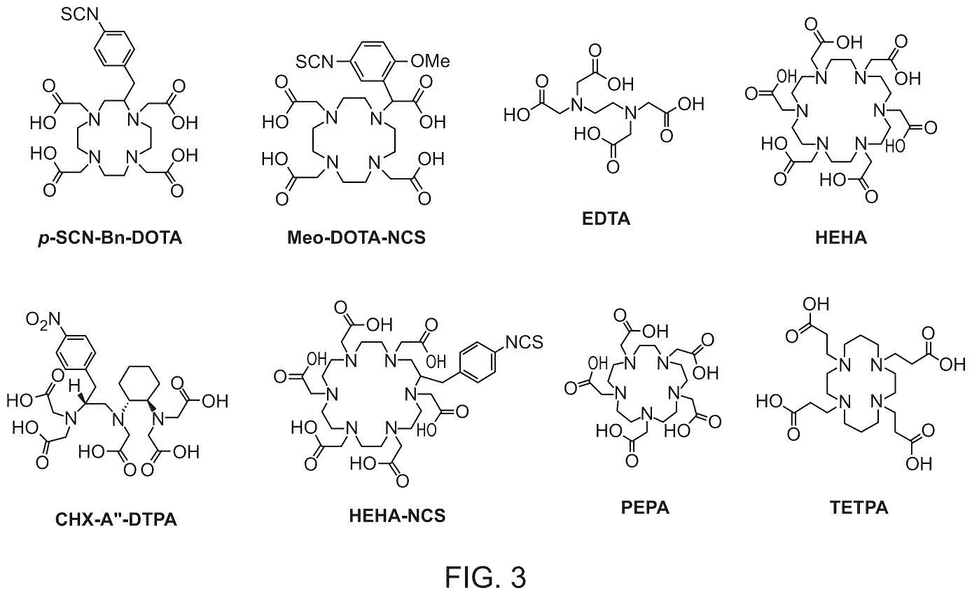

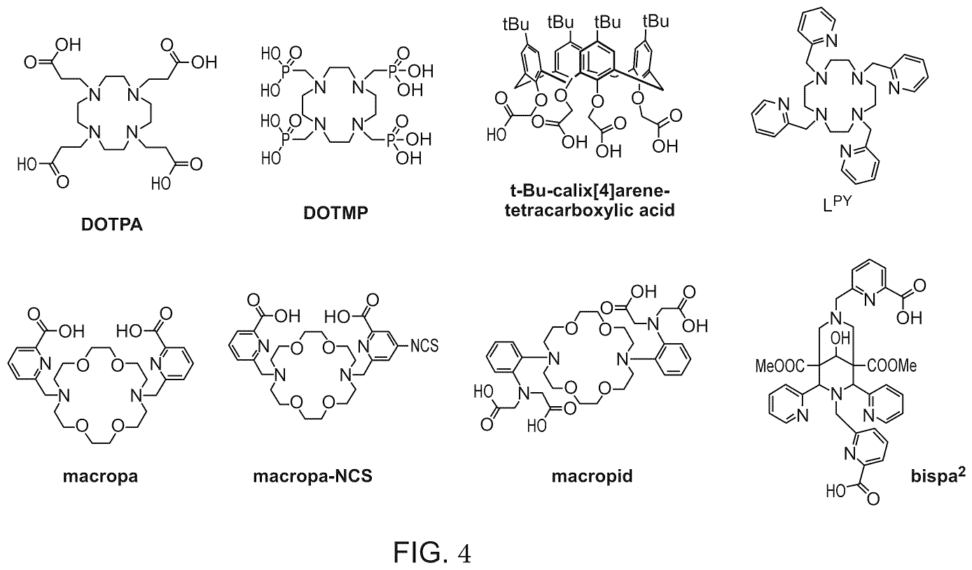

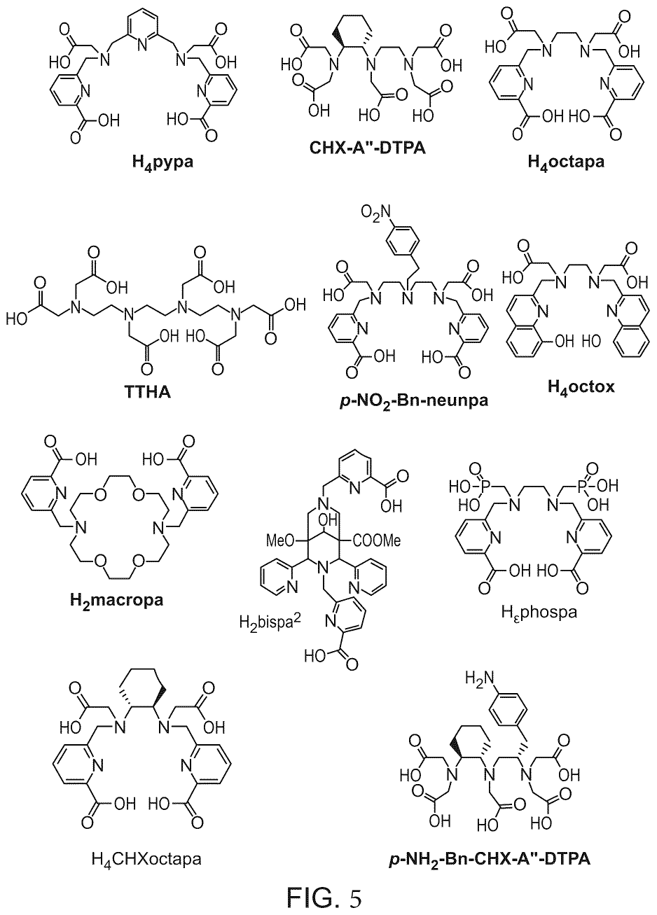

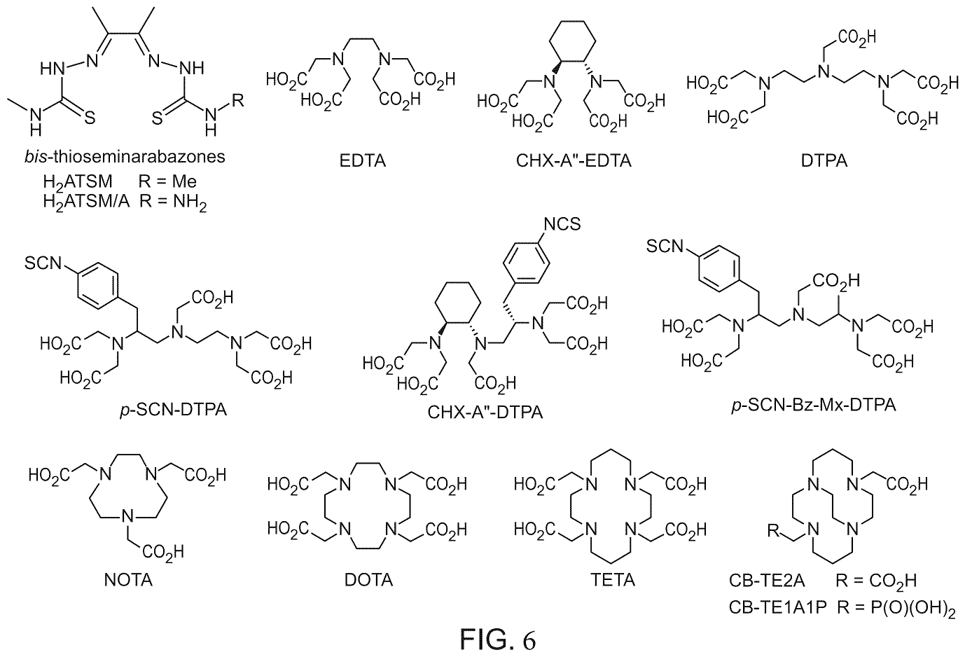

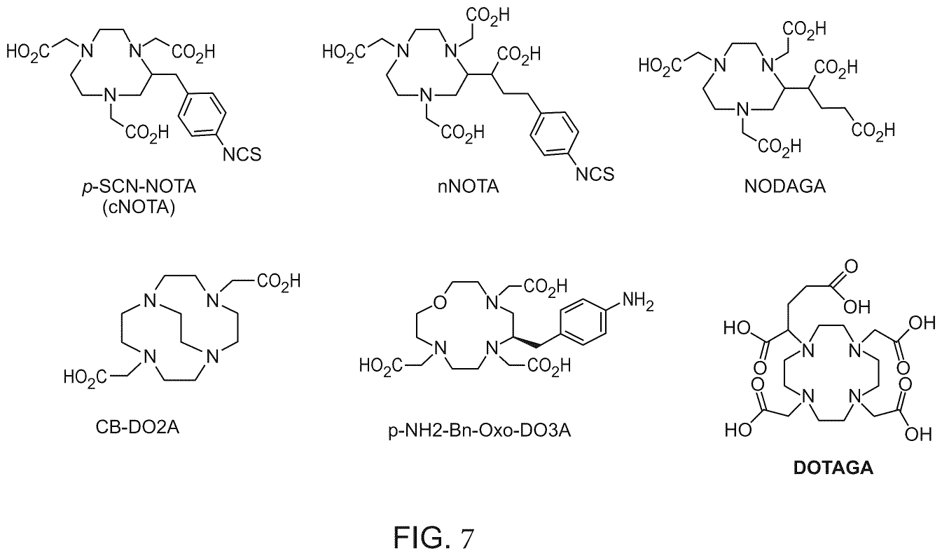

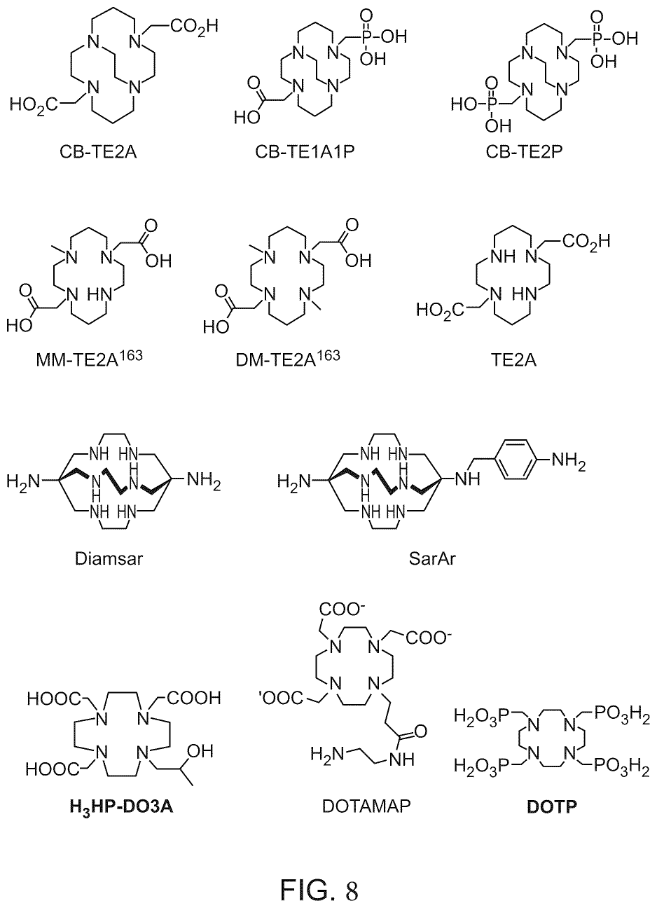

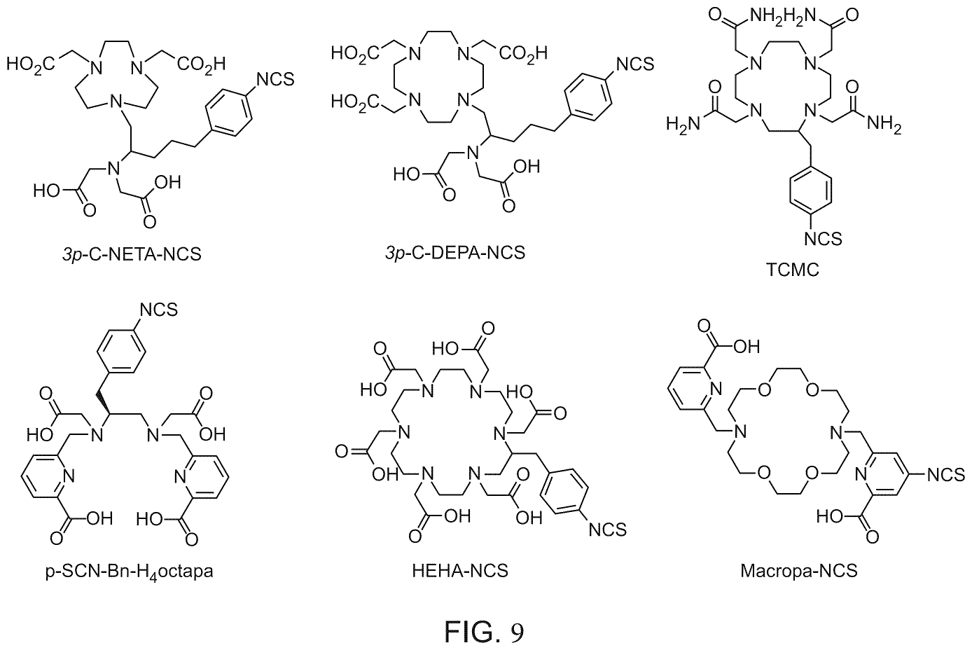

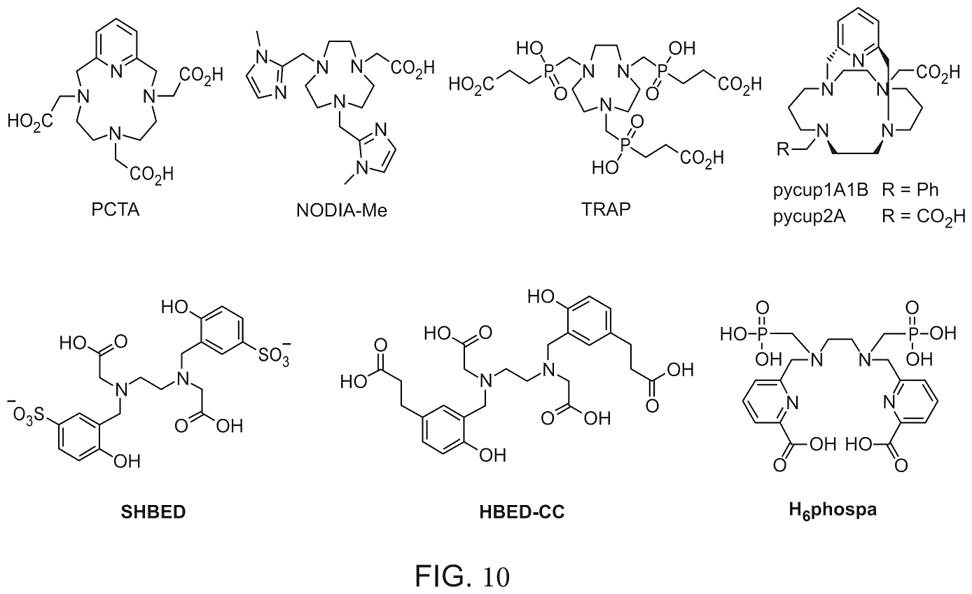

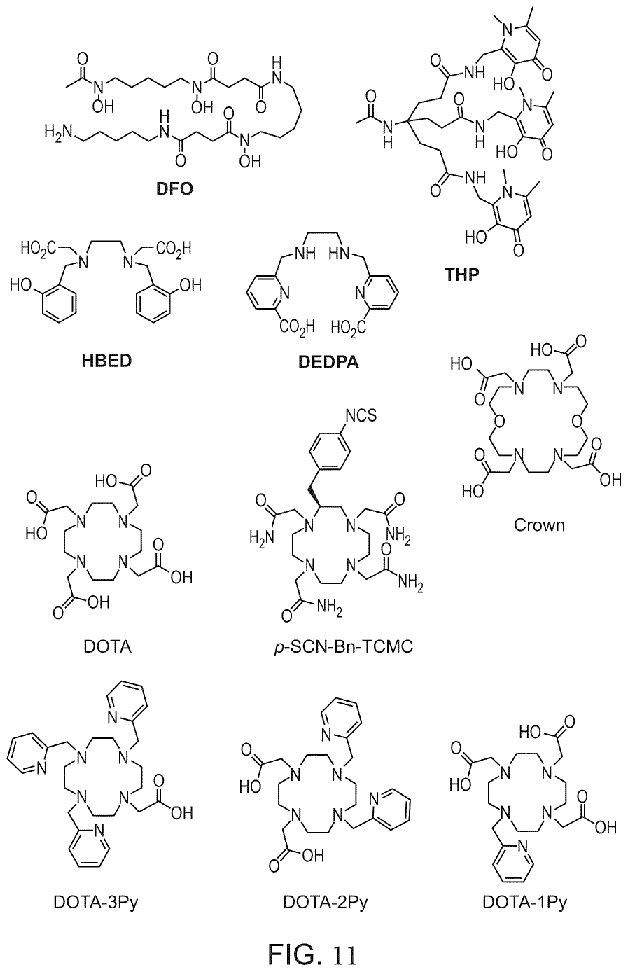

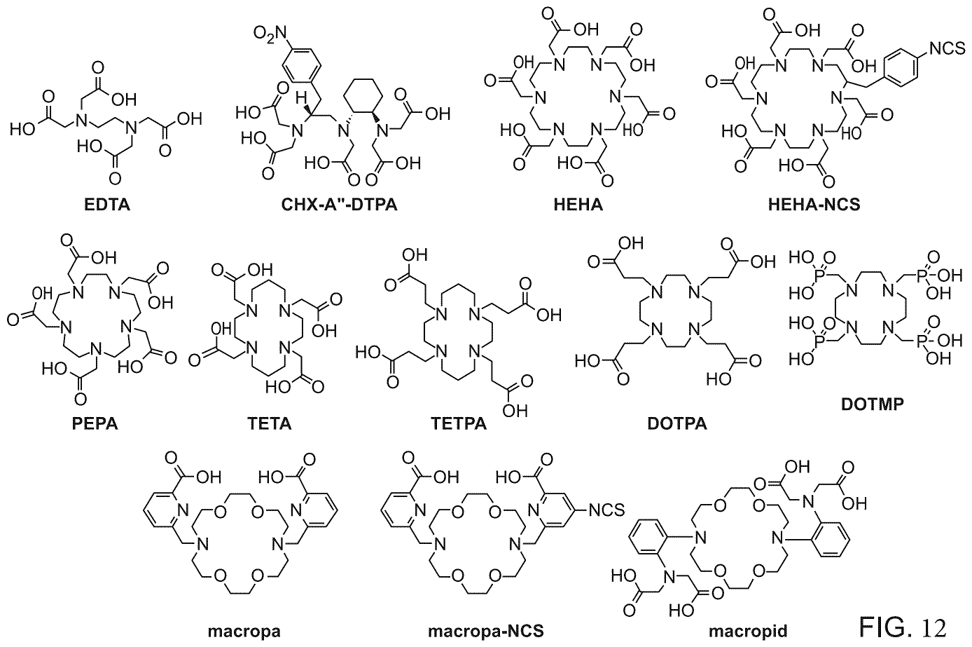

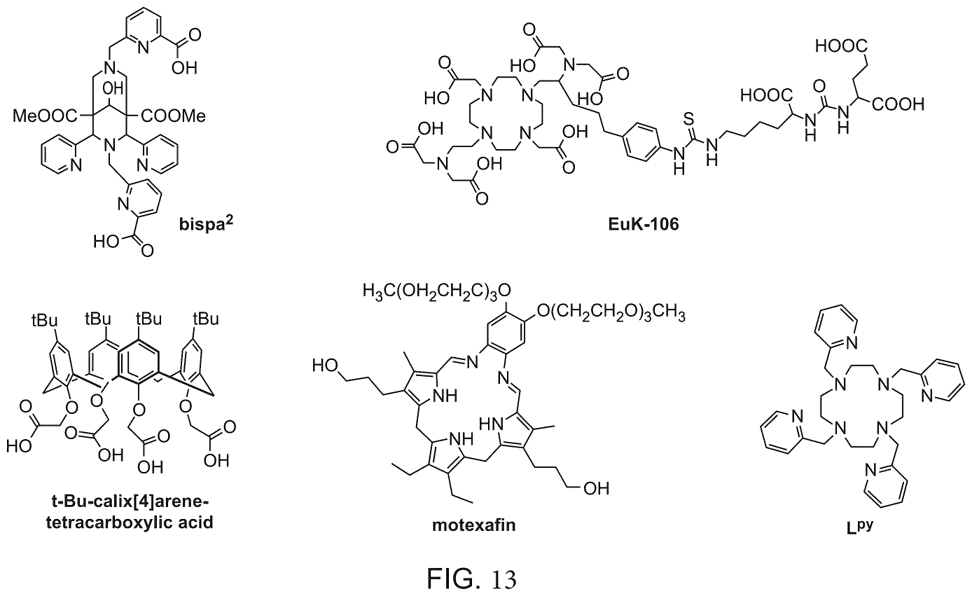

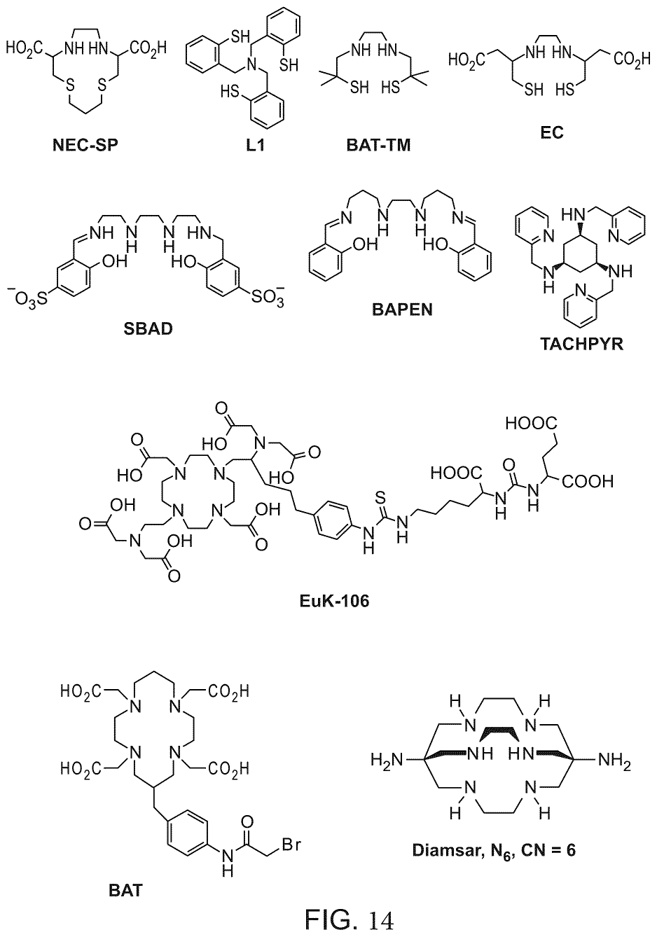

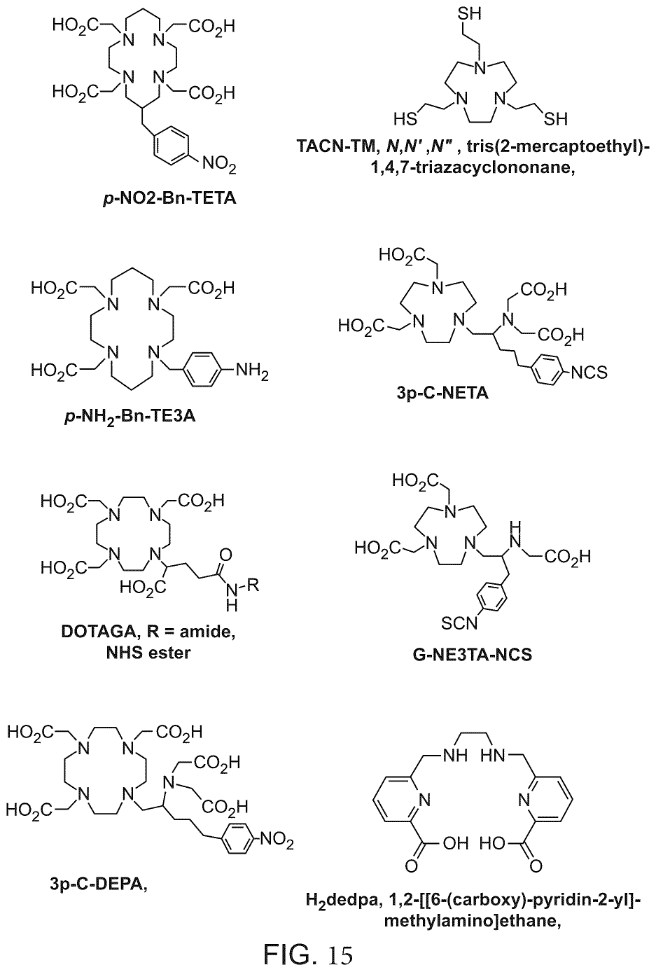

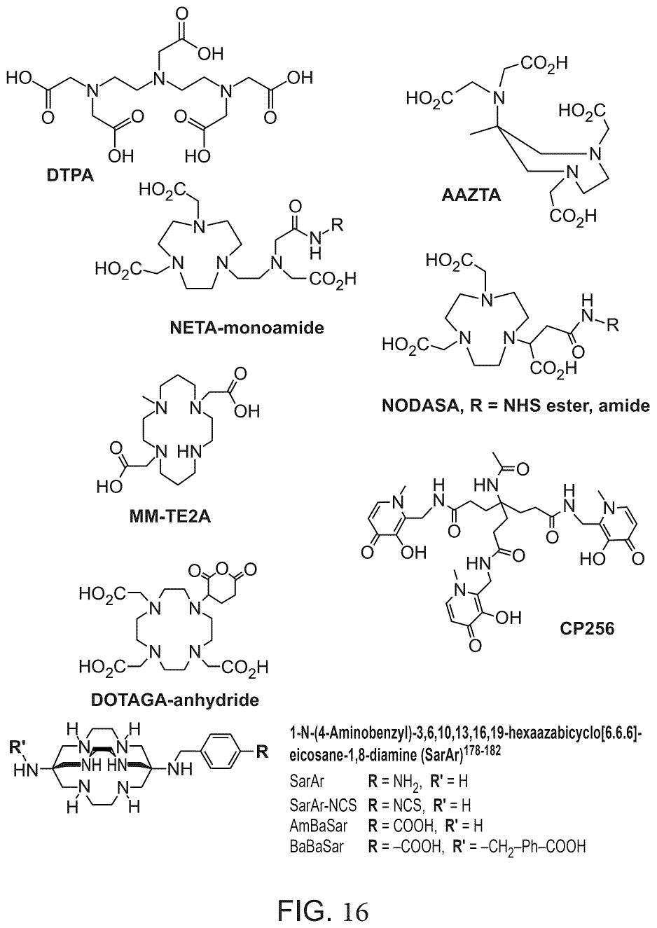

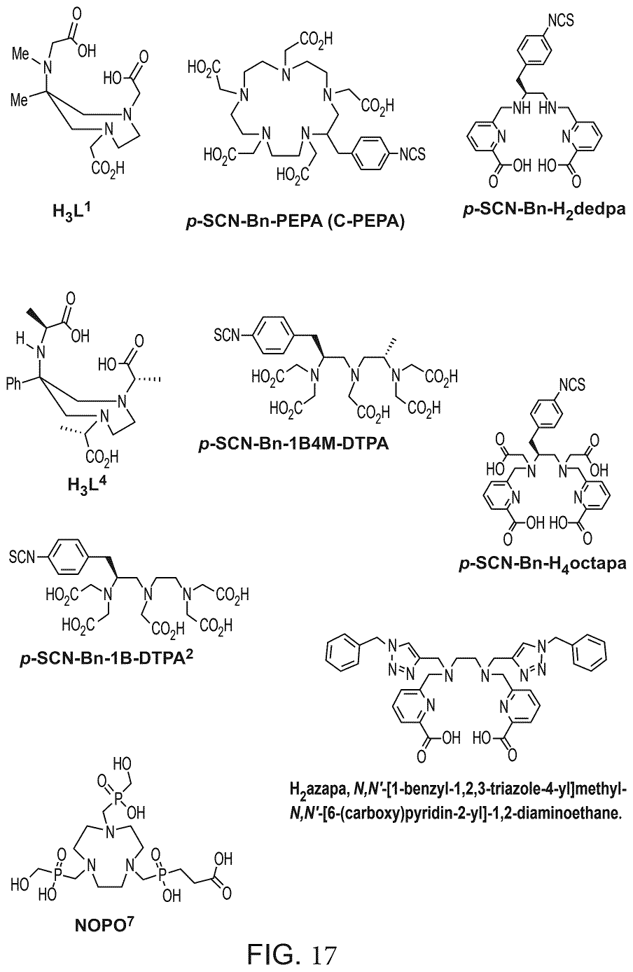

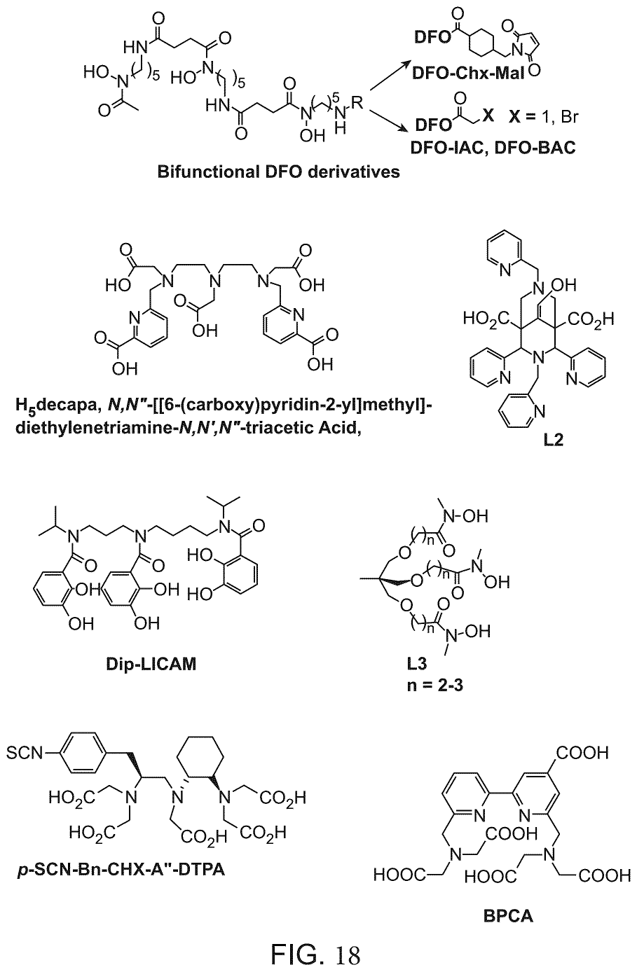

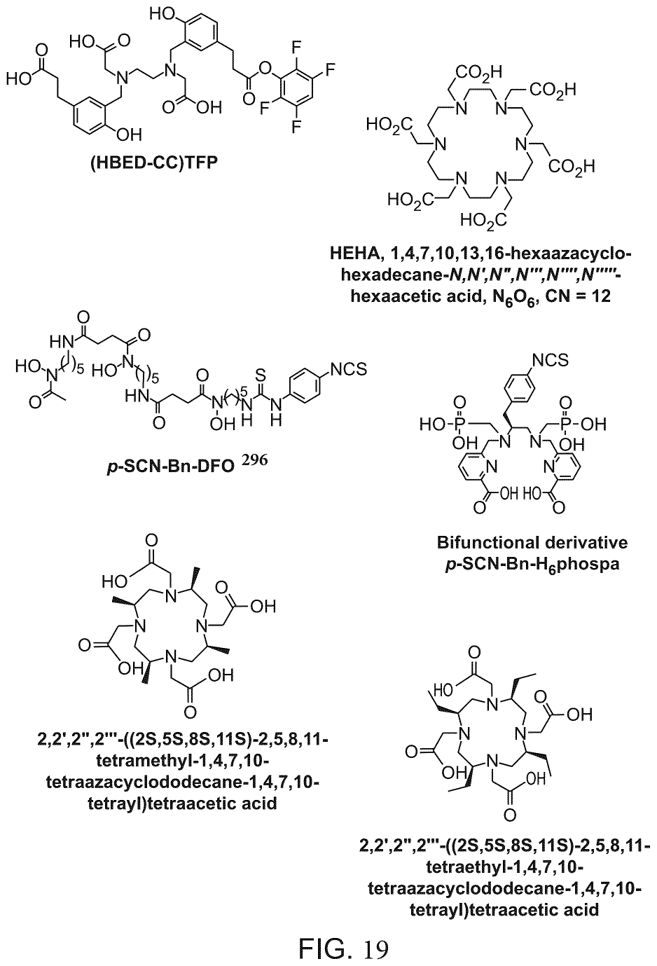

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “figure” and “FIG.” herein), of which: FIG. 1 to FIG. 19 illustrate the structures of representative metal chelators.

### DETAILED DESCRIPTION

### Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

The use of the word “a” or “an” when used in conjunction with the term “comprising” in the claims and/or the description can mean “one”, but it is also consistent with the meaning of “one or more”, “at least one” and “one or more than one”.

As used herein, the terms “comprising” and “including” can be used interchangeably. The terms “comprising” and “including” are to be interpreted as specifying the presence of the stated features or components as referred to, but do not preclude the presence of additional features or components. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: A; B; C; A and B; A and C; B and C; A, B and C. An exception to this definition will occur only when a combination of elements, functions, steps or acts are in some way inherently mutually exclusive.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means that the numeric value or range of values may vary within 25%, 20%, 15%, 10%, 9%, 8%, 7%, 6%, 5%, 4%, 3%, 2%, 1.5%, 1%, 0.5%, or 0.25% of the recited value or range of values. In one embodiment, the term “about” refers to a value that is no more than 10% above or below the value being modified by the term. For example, the term “about 10 mg/m²” means a range of from 9 mg/m² to 11 mg/m².

The term “optional” or “optionally” means that the subsequently described circumstance may or may not occur, so that the description includes instances wherein the circumstance occurs, and instances wherein the circumstance does not occur.

Unless specifically stated otherwise, where a compound may assume alternative tautomeric, regioisomeric and/or stereoisomeric forms, all alternative isomers are intended to be encompassed within the scope of the claimed subject matter. For example, where a compound has one or more chiral centers, it can occur as racemate, individual enantiomer or diastereomer, or mixture thereof; it is intended that all such isomeric forms be encompassed herein. Where a compound can have one or more tautomeric forms, it is intended that all tautomers be encompassed herein.

Thus, the compounds provided herein may be enantiomerically pure, or be stereoisomeric or diastereomeric mixtures. As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A typical stereomerically pure compound comprises greater than about 80% by weight of one stereoisomer of the compound and less than about 20% by weight of the other stereoisomers of the compound, in one embodiment greater than about 90% by weight of one stereoisomer of the compound and less than about 10% by weight of the other stereoisomers of the compound, in one embodiment greater than about 95% by weight of one stereoisomer of the compound and less than about 5% by weight of the other stereoisomers of the compound, and in one embodiment greater than about 97% by weight of one stereoisomer of the compound and less than about 3% by weight of the other stereoisomers of the compound. A stereomerically pure Compound A, A-S, or A-R used herein comprises greater than about 85% by weight of one stereoisomer of the compound, in one embodiment greater than about 90% by weight of one stereoisomer of the compound, in one embodiment greater than about 95% by weight of one stereoisomer of the compound, and in one embodiment greater than about 97% by weight of one stereoisomer of the compound. As used herein and unless otherwise indicated, the term “stereomerically enriched” means a composition that comprises greater than about 60% by weight of one stereoisomer of a compound, in one embodiment greater than about 70% by weight of one stereoisomer of a compound, and in one embodiment greater than about 80% by weight of one stereoisomer of a compound. As used herein and unless otherwise indicated, the term “enantiomerically pure” means a stereomerically pure composition of a compound having one chiral center. Similarly, the term “enantiomerically enriched” means a stereomerically enriched composition of a compound having one chiral center. As used herein, stereoisomeric or diastereomeric mixtures means a composition that comprises more than one stereoisomer of a compound. A typical stereoisomeric mixture of a compound comprises about 50% by weight of one stereoisomer of the compound and about 50% by weight of the other stereoisomers of the compound, or comprises greater than about 50% by weight of one stereoisomer of the compound and less than about 50% by weight of the other stereoisomers of the compound, or comprises greater than about 45% by weight of one stereoisomer of the compound and less than about 55% by weight of the other stereoisomers of the compound, or comprises greater than about 40% by weight of one stereoisomer of the compound and less than about 60% by weight of the other stereoisomers of the compound, or comprises greater than about 35% by weight of one stereoisomer of the compound and less than about 65% by weight of the other stereoisomers of the compound.

It is to be understood that the compounds provided herein may contain chiral centers. Such chiral centers may be of either the (R) or(S) configuration, or may be a mixture thereof. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its(S) form.

Optically active (+) and (−), (R)- and(S)—, or (D)- and (L)-isomers may be prepared using chiral synthons or chiral reagents, or resolved using conventional techniques, such as chromatography on a chiral stationary phase.

A compound provided herein can contain unnatural proportions of atomic isotopes at one or more of its atoms. For example, the compound may be radiolabeled with radioactive isotopes, such as for example tritium (³H) or carbon-14 (¹⁴C), or may be isotopically enriched, such as with deuterium (D or ²H), carbon-13 (¹³C), or nitrogen-15 (¹⁵N). As used herein, an “isotopolog” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., non-Hodgkin lymphoma therapeutic agents, research reagents, e.g., binding assay reagents, and diagnostic agents, e.g., in vivo imaging agents. All isotopic variations of the compounds described herein, whether radioactive or not, are intended to be encompassed within the scope of the embodiments described herein. In some embodiments, provided herein are isotopologues of the compounds, for example, the isotopologues are deuterium, carbon-13 (¹³C), and/or nitrogen-15 (¹⁵N) enriched compounds.

As used herein, “deuterated”, means a compound wherein at least one hydrogen (H) has been replaced by deuterium (D or ²H), that is, the compound is enriched in deuterium in at least one position. In some embodiments, isotopologues provided herein are deuterium enriched compounds. In some embodiments, isotopologues provided herein are deuterium enriched compounds, where the deuteration occurs on the chiral center.

In the description herein, if there is any discrepancy between a chemical name and a chemical structure, the structure controls.

As used herein and unless otherwise indicated, the term “solvate” means a compound provided herein or a salt thereof, that further includes a stoichiometric or non-stoichiometric amount of solvent bound by non-covalent intermolecular forces. Where the solvent is water, the solvate is a hydrate.

As used herein and unless otherwise specified, the term “pharmaceutically acceptable salt” encompasses non-toxic acid and base addition salts of the compound to which the term refers. Acceptable non-toxic acid addition salts include those derived from organic and inorganic acids known in the art, which include, for example, hydrochloric acid, hydrobromic acid, phosphoric acid, sulfuric acid, methanesulfonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N′-dibenzylethylenediamine, chloroprocaine, choline, diethanolamine, ethylenediamine, meglumine (N-methylglucamine), lysine, and procaine.

It is understood that, independently of stereomerical or isotopic composition, each compound provided herein can be provided in the form of any of the pharmaceutically acceptable salts provided herein. Equally, it is understood that the isotopic composition may vary independently from the stereomerical composition of each compound provided herein. Further, the isotopic composition, while being restricted to those elements present in the respective compound or salt thereof, may otherwise vary independently from the selection of the pharmaceutically acceptable salt of the respective compound.

As used herein and unless otherwise specified, an “alkyl” group is a saturated, straight chain or branched, non-cyclic hydrocarbon having from 1 to 10 carbon atoms, typically from 1 to 8 carbons or, in some embodiments, from 1 to 6, 1 to 4, 1 to 3, or 2 to 6 carbon atoms. Representative straight chain alkyl groups include-methyl, -ethyl, -n-propyl, -n-butyl, -n-pentyl and -n-hexyl; while branched alkyls include-isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like.

Alkyl groups can be substituted or unsubstituted. When the alkyl groups described herein are said to be “substituted,” they may be substituted with any substituent or substituents as those found in the exemplified compounds and embodiments disclosed herein, as well as halogen; hydroxy; alkoxy; cycloalkyloxy, aryloxy, heterocyclyloxy, heteroaryloxy, heterocycloalkyoxy, cycloalkylalkyloxy, aralkyloxy, heterocyclylalkyloxy, heteroarylalkyloxy, heterocycloalkyalkyloxy; oxo (═O); amino, alkylamino, cycloalkylamino, arylamino, heterocyclylamino, heteroarylamino, heterocycloalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In some embodiments, one or more hydrogens, such as one, two, three, four, or five hydrogens, in an alkyl, alkenyl, or alkynyl group may be replaced with halogen.

As used herein and unless otherwise specified, a “cycloalkyl” group is a saturated, or partially saturated cyclic hydrocarbon having from 3 to 10 carbon atoms in a single ring or multiple condensed, spiro, or bridged rings, which can be optionally substituted. In some embodiments, a cycloalkyl group has 3 to 8 ring carbon atoms, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. In some embodiments, a cycloalkyl is saturated.

As used herein and unless otherwise specified, an “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryl groups include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

As used herein and unless otherwise specified, “hetero” refers to an atom that is not carbon or hydrogen. Suitable heteroatoms include oxygen, sulfur, and nitrogen.

As used herein and unless otherwise specified, a “heteroaryl” group is an aromatic ring system having one to four heteroatoms as ring atoms in a heteroaromatic ring system, wherein the remainder of the ring atoms are carbon atoms. In some embodiments, heteroaryl groups contain 3 to 6 ring atoms, and in others from 5 to 9, 6 to 9, 5 to 10, or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indol-2-onyl), isoindolin-1-onyl, azaindolyl, pyrrolopyridyl (e.g., 1H-pyrrolo[2,3-b]pyridyl), indazolyl, benzimidazolyl (e.g., 1H-benzo[d]imidazolyl), azabenzimidazolyl, imidazopyridyl (e.g., 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl, 3,4-dihydroisoquinolin-1 (2H)-onyl, tetrahydroquinolinyl, quinoxalinyl, and quinazolinyl groups. A heteroaryl group can be substituted or unsubstituted. The phrase “heteroaryl groups” also includes groups containing fused rings, such as fused heteroaromatic-aliphatic or fused heteroaromatic-heteroaliphatic ring systems.

As used herein and unless otherwise specified, a “heterocyclyl” or “heterocycloalkyl” is a cycloalkyl in which one to four of the ring carbon atoms are independently replaced with a heteroatom. Suitable heteroatoms include oxygen, sulfur and nitrogen. In some embodiments, heterocyclyl groups include 3 to 10 ring members, whereas other such groups have 3 to 5, 3 to 6, or 3 to 8 ring members. In some embodiments, a heterocyclyl group has 4 to 7 ring members. A heterocyclyl group can be substituted or unsubstituted. When a heterocyclyl is substituted, a substituent can be bonded to the heterocyclyl at any ring atom (i.e., at any carbon atom or heteroatom of the heterocyclic ring). The phrase includes fused, spiro, and bridged polycyclic ring systems containing a heteroatom such as, but not limited to, quinuclidyl. Representative examples of a heterocyclyl group include, but are not limited to, aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2 (1H)-one. In some embodiments, heterocyclyl is saturated.

As used herein and unless otherwise specified, a “cycloalkylalkyl” group is a radical of the formula: -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopropylmethyl, cyclobutylmethyl, cyclopentylmethyl, cyclohexylmethyl, cyclopropylethyl, cyclobutylethyl, cyclopentylethyl, cyclohexylethyl, cyclopentylpropyl, cyclohexylpropyl and the like.

As used herein and unless otherwise specified, an “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and aralkyl groups wherein the aryl group is fused to a cycloalkyl group such as indan-4-yl ethyl.

As used herein and unless otherwise specified, a “heterocyclylalkyl” group is a radical of the formula: -alkyl-heterocyclyl, wherein alkyl and heterocyclyl are defined above. A “heteroarylalkyl” group is a radical of the formula: -alkyl-heteroaryl, wherein alkyl and heteroaryl are defined above. Substituted heterocyclylalkyl groups may be substituted at the alkyl, the heterocyclyl, or both the alkyl and the heterocyclyl portions of the group. Representative heterocylylalkyl groups include but are not limited to morpholin-4-yl ethyl, morpholin-4-yl propyl, furan-2-yl methyl, furan-3-yl methyl, pyridin-3-yl methyl, tetrahydrofuran-2-yl ethyl, and indol-2-yl propyl.

As used herein and unless otherwise specified, “halogen” or “halo” is fluorine, chlorine, bromine, or iodine. In some embodiments, halogen is fluorine, chlorine, or bromine. In some embodiments, halogen is fluorine or chlorine. In some embodiments, halogen is fluorine or bromine. In some embodiments, halogen is chlorine or bromine.

As used herein and unless otherwise specified, a “haloalkyl” group is an alkyl group as described above substituted with one or more independently selected halogens, wherein halogen is as described above. In some embodiments, haloalkyl is perhalogenated. Examples of haloalkyl include but are not limited to difluoromethyl, trifluoromethyl, chlorofluoromethyl, and the like.

As used herein and unless otherwise specified, a “hydroxyalkyl” group is an alkyl group as described above substituted with one or more hydroxy groups.

As used herein and unless otherwise specified, an “alkoxy” group is —O-(alkyl), wherein alkyl is defined above. An “alkylthio” group is —S-(alkyl), wherein alkyl is defined above.

As used herein and unless otherwise specified, an “alkoxyalkyl” group is -(alkyl)-O-(alkyl), wherein alkyl is defined above.

As used herein and unless otherwise specified, a “cycloalkyloxy” group is —O-(cycloalkyl), wherein cycloalkyl is defined above.

As used herein and unless otherwise specified, an “aryloxy” group is —O-(aryl), wherein aryl is defined above.

As used herein and unless otherwise specified, a “heterocyclyloxy” group is —O-(heterocyclyl), wherein heterocyclyl is defined above. A “heteroaryloxy” group is —O-(heteroaryl), wherein heteroaryl is defined above.

As used herein and unless otherwise specified, an “amino” group is a radical of the formula: —NH₂, —NH(R^{#}), or —N(R^{#})₂, wherein each R^{#} is independently an alkyl, cycloalkyl, cycloalkylalkyl, aryl, aralkyl, heterocyclyl (e.g., heteroaryl or heterocycloalkyl), or heterocyclylalkyl (e.g., heteroarylalkyl or heterocycloalkylalkyl) group defined above, each of which is independently substituted or unsubstituted.

In one embodiment, an “amino” group is an “alkylamino” group, which is a radical of the formula: —NH-alkyl or —N(alkyl)₂, wherein each alkyl is independently defined above. The term “cycloalkylamino”, “arylamino”, “heterocyclylamino”, “heteroarylamino”, “heterocycloalkylamino”, or the like, mirrors the above description for “alkylamino” where the term “alkyl” is replaced with “cycloalkyl”, “aryl”, “heterocyclyl”, “heteroaryl”, “heterocycloalkyl”, or the like, respectively.

As used herein and unless otherwise specified, a “carboxy” group is a radical of the formula: —C(O)OH.

As used herein and unless otherwise specified, an “acyl” group is a radical of the formula: —C(O)(R^{#}) or —C(O)H, wherein R^{#} is defined above. A “formyl” group is a radical of the formula: —C(O)H.

As used herein and unless otherwise specified, an “amido” group is a radical of the formula: —C(O)—NH₂, —C(O)—NH(R^{#}), —C(O)—N(R^{#})₂, —NH—C(O)H, —NH—C(O)—(R^{#}), —N(R^{#})—C(O)H, or —N(R^{#})—C(O)—(R^{#}), wherein each R^{#} is independently defined above.

In one embodiment, an “amido” group is an “aminocarbonyl” group, which is a radical of the formula: —C(O)—NH₂, —C(O)—NH(R^{#}), —C(O)—N(R^{#})₂, wherein each R^{#} is independently defined above.

In one embodiment, an “amido” group is an “acylamino” group, which is a radical of the formula: —NH—C(O)H, —NH—C(O)—(R^{#}), —N(R^{#})—C(O)H, or —N(R^{#})—C(O)—(R^{#}), wherein each R^{#} is independently defined above.

As used herein and unless otherwise specified, a “sulfonylamino” group is a radical of the formula: —NHSO₂(R^{#}) or —N(R^{#})SO₂(R^{#}), wherein each R^{#} is defined above.

As used herein and unless otherwise specified, an “ester” group is a radical of the formula: —C(O)—O—(R^{#}) or —O—C(O)—(R^{#}), wherein R^{#} is defined above.

In one embodiment, an “ester” group is an “alkoxycarbonyl” group, which is a radical of the formula: —C(O)—O-(alkyl), wherein alkyl is defined above. The term “cycloalkyloxycarbonyl”, “aryloxycarbonyl”, “heterocyclyloxycarbonyl”, “heteroaryloxycarbonyl”, “heterocycloalkyloxycarbonyl”, or the like, mirrors the above description for “alkoxycarbonyl” where the term “alkoxy” is replaced with “cycloalkyloxy”, “aryloxy”, “heterocyclyloxy”, “heteroaryloxy”, “heterocycloalkyloxy”, or the like, respectively.

As used herein and unless otherwise specified, a “carbamate” group is a radical of the formula: —O—C(O)—NH₂, —O—C(O)—NH(R^{#}), —O—C(O)—N(R^{#})₂, —NH—C(O)—O—(R^{#}), or —N(R^{#})—C(O)—O—(R^{#}), wherein each R^{#} is independently defined above.

As used herein and unless otherwise specified, a “urea” group is a radical of the formula: —NH(CO)NH₂, —NHC(O)NH(R^{#}), —NHC(O)N(R)₂, —N(R^{#})C(O)NH₂, —N(R^{#})C(O)NH(R^{#}), or —N(R^{#})C(O)N(R^{#})₂, wherein each R^{#} is independently defined above.

As used herein and unless otherwise specified, a “sulfinyl” group is a radical of the formula: —S(O)R^{#}, wherein R^{#} is defined above.

As used herein and unless otherwise specified, a “sulfonyl” group is a radical of the formula: —S(O)₂R^{#}, wherein R^{#} is defined above.

As used herein and unless otherwise specified, an “aminosulfonyl” group is a radical of the formula: —SO₂NH₂, —SO₂NH(R^{#}), or —SO₂N (R^{#})₂, wherein each R^{#} is independently defined above.

When the groups described herein, with the exception of alkyl groups, are said to be “substituted,” they may be substituted with any appropriate substituent or substituents. Illustrative examples of substituents are those found in the exemplary compounds and embodiments disclosed herein, as well as halogen; alkyl, alkenyl, alkynyl, cycloalkyl, aryl, heterocyclyl, heteroaryl, cycloalkylalkyl, aralkyl, heterocyclylalkyl, heteroarylalkyl, optionally further substituted; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; oxo (═O); oxide (e.g., a nitrogen atom substituted with an oxide is called N-oxide); amino, alkylamino, cycloalkylamino, arylamino, heterocyclylamino, heteroarylamino, imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In some embodiments, one or more hydrogens, such as one, two, three, four, or five hydrogens, in a substituent may be replaced with halogen. In some embodiments, substitution replaces a hydrogen atom with alkyl, alkoxy, aryloxy, halogen, or haloalkyl.

“Tautomers” refers to isomeric forms of a compound that are in equilibrium with each other through the migration of a proton. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

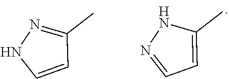

As readily understood by one skilled in the art, a wide variety of functional groups and other structures may exhibit tautomerism. Unless otherwise specified, all tautomeric forms of the compounds of the present disclosure are within the scope of the present disclosure.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself.

The term “therapeutically effective amount” means an amount capable of treating a disorder, disease or condition, or symptoms thereof.

The term “effective amount” includes a therapeutically effective amount, as well as an amount useful for imaging and/or diagnosing a disorder, disease or condition, or symptoms thereof.

The term “subject” or “patient” includes humans. In some embodiments, the subject is a human.

Unless otherwise defined, the technical and scientific terms used herein have the same meaning as commonly understood by one of ordinary skill in the art to which this application belongs.

### Compounds

The present disclosure provides certain compounds that comprise a targeting ligand that binds to a CA-IX protein. In some embodiments, the compounds described herein comprise a targeting ligand that binds to a CA-IX protein and a metal chelator that is configured to bind with a radionuclide. In some embodiments, the compounds described herein comprise a radionuclide.

The present disclosure further provides certain compounds that comprise a targeting ligand that binds to a CA-IX protein and an additional ligand that binds to albumin. In some embodiments, an additional ligand that binds to albumin reduces uptake of such compounds in the stomach.

Disclosed herein is a compound of the following structural formula:

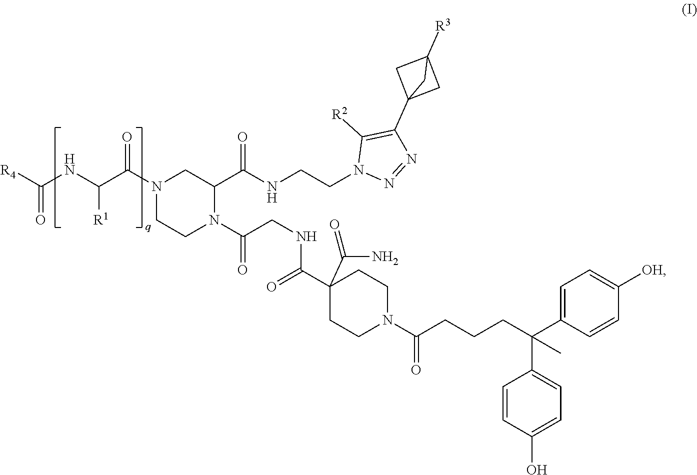

or a pharmaceutically acceptable salt thereof, wherein:
q is 0 or 1;
R¹ is

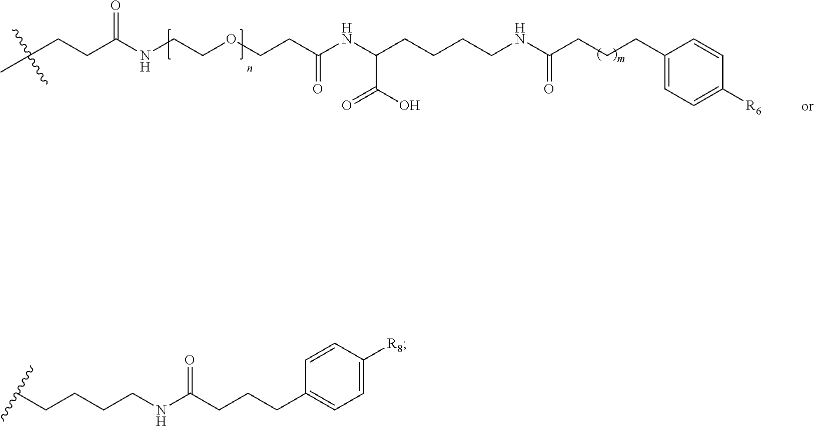

n is 2, 3, 4, 5, 6, 7, 8, 9, 10, 11, 12, 13, 14, 15, or 16;
m is 1, 2, or 3;
   R₆ is H, halogen, —CH₃, —OCH₃, or —OCH₂CH₃;
R₈ is halogen or —CH₃;
R² is H or —COOH;
R³ is

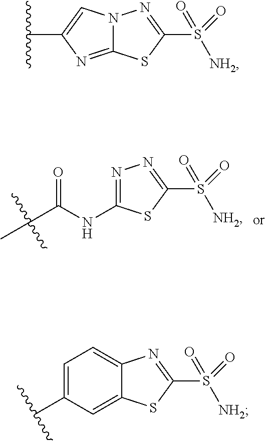 [SMILES: *c1cn2nc(S(N)(=O)=O)sc2n1 ; *C(=O)Nc1nnc(S(N)(=O)=O)s1 ; "or" ; *c1ccc2nc(S(N)(=O)=O)sc2c1]

and
R⁴ comprises a metal chelator (CL).
   It is understood that although formula (I) is shown without specific stereochemistry, the structure of formula (I) encompasses stereoisomers thereof, such as formula (Ia) and (Ib), as well as mixtures of stereoisomers thereof.

In some embodiments, q is 0. In some embodiments, q is 1.

Also disclosed herein is a compound of the following structural formula:

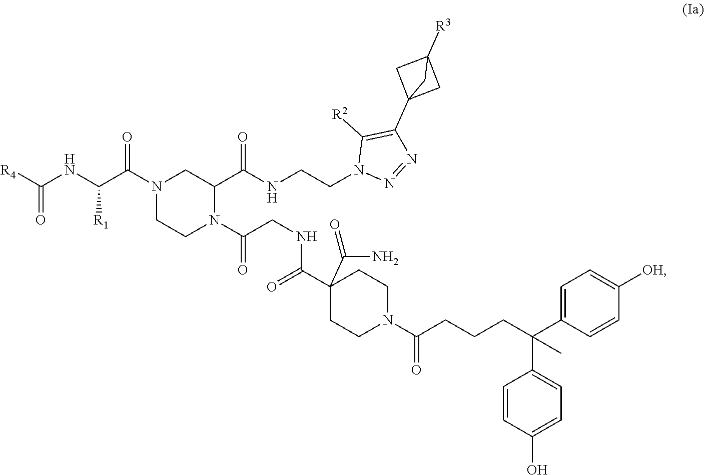

or a pharmaceutically acceptable salt thereof.

Also disclosed herein is a compound of the following structural formula:

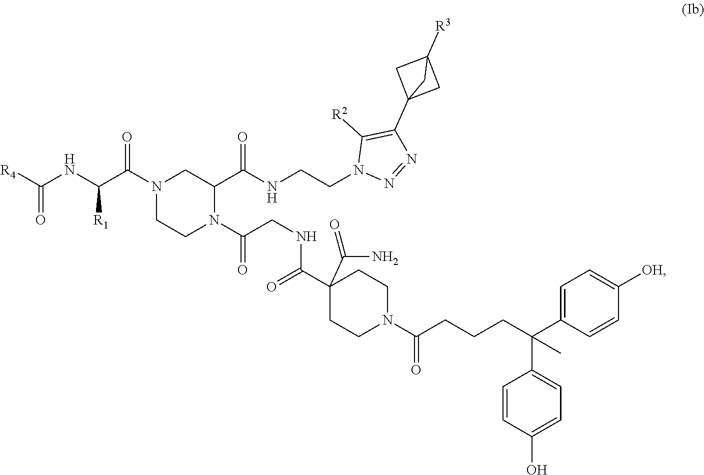

or a pharmaceutically acceptable salt thereof.

In some embodiments, R³ is

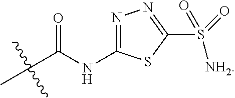 [SMILES: *C(=O)Nc1nnc(S(N)(=O)=O)s1]

In some embodiments, R³ is

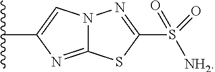 [SMILES: *c1cn2nc(S(N)(=O)=O)sc2n1]

In some embodiments, R³ is

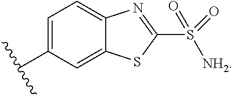 [SMILES: *c1ccc2nc(S(N)(=O)=O)sc2c1]

In some embodiments, R⁴ is

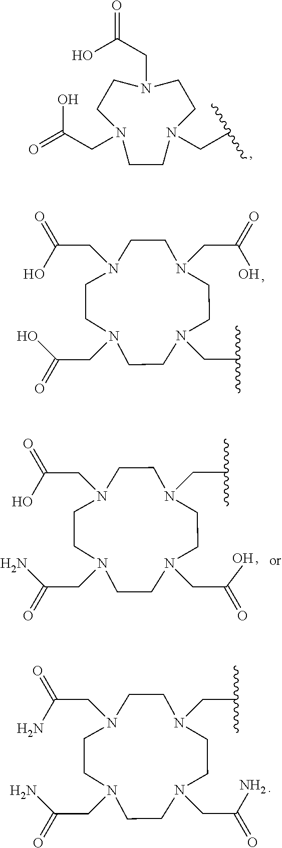 [SMILES: *CN1CCN(CC(=O)O)CCN(CC(=O)O)CC1 ; *CN1CCN(CC(=O)O)CCN(CC(=O)O)CCN(CC(=O)O)CC1 ; *CN1CCN(CC(=O)O)CCN(CC(N)=O)CCN(CC(=O)O)CC1 ; *CN1CCN(CC(N)=O)CCN(CC(N)=O)CCN(CC(N)=O)CC1]

In some embodiments, R⁴ is

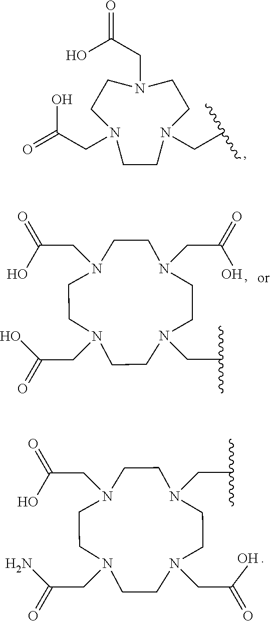 [SMILES: *CN1CCN(CC(=O)O)CCN(CC(=O)O)CC1 ; *CN1CCN(CC(=O)O)CCN(CC(=O)O)CCN(CC(=O)O)CC1 ; *CN1CCN(CC(=O)O)CCN(CC(N)=O)CCN(CC(=O)O)CC1]

In some embodiments, R⁴ is

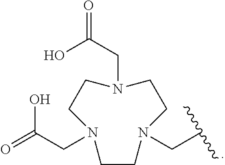 [SMILES: *CN1CCN(CC(=O)O)CCN(CC(=O)O)CC1]

In some embodiments, R⁴ is

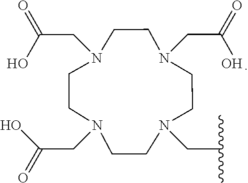 [SMILES: *CN1CCN(CC(=O)O)CCN(CC(=O)O)CCN(CC(=O)O)CC1]

In some embodiments, R⁴ is

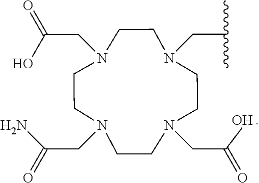 [SMILES: *CN1CCN(CC(=O)O)CCN(CC(N)=O)CCN(CC(=O)O)CC1]

In some embodiments, R⁴ is

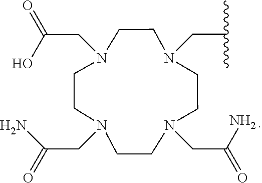 [SMILES: *CN1CCN(CC(N)=O)CCN(CC(N)=O)CCN(CC(=O)O)CC1]

In some embodiments, R² is H. In alternative embodiments, R² is —COOH.

In some embodiments, R¹ is

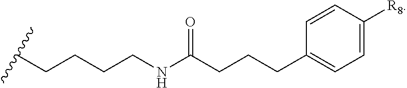

In alternative embodiments, R¹ is

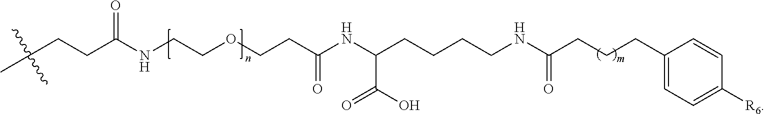

In more particular embodiments, R¹ is

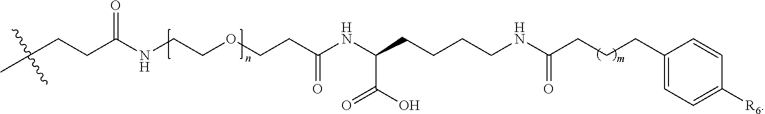

In other particular embodiments, R¹ is

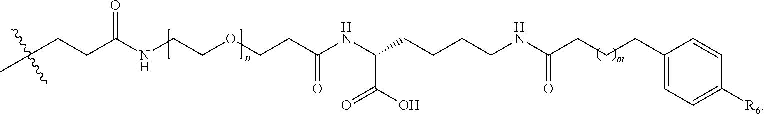

In some embodiments, R₈ is —CH₃. In some embodiments, R₈ is halogen. In some embodiments, R₈ is F. In some embodiments, R₈ is Cl. In some embodiments, R₈ is I. In some embodiments, R⁸ is Br.

In some embodiments, n is 6, 8, or 16. In some embodiments, n is 6. In some embodiments, n is 8. In some embodiments, n is 16.

In some embodiments, m is 1. In some embodiments, m is 2. In some embodiments, m is 3.

In some embodiments, R⁶ is H. In some embodiments, R⁶ is a halogen. In some embodiments, R⁶ is F. In some embodiments, R⁶ is Cl. In some embodiments, R⁶ is Br. In some embodiments, R⁶ is I. In some embodiments, R⁶ is —CH₃. In some embodiments, R⁶ is —OCH₃. In some embodiments, R⁶ is —OCH₂CH₃.

Disclosed herein is a compound of the following structural formula:

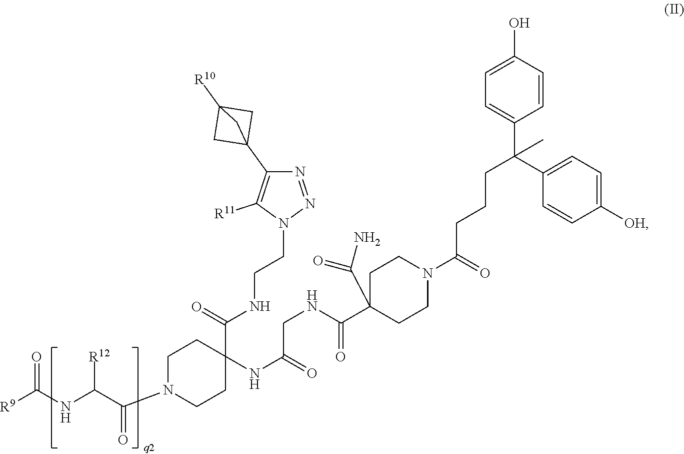

or a pharmaceutically acceptable salt thereof, wherein:
q2 is 0 or 1;
R¹² is

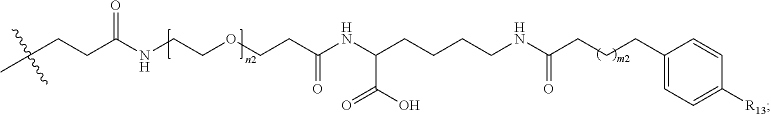

n2 is 2, 3, 4, 5, 6, 7, 8, 9, 10, 11, 12, 13, 14, 15, or 16;
m2 is 1, 2, or 3;
   R¹³ is H, halogen, —CH₃, —OCH₃, or —OCH₂CH₃;
R¹¹ is H or —COOH;
R¹⁰ is

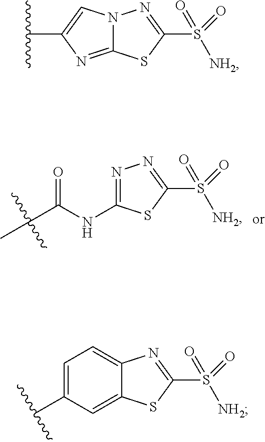 [SMILES: *c1cn2nc(S(N)(=O)=O)sc2n1 ; *C(=O)Nc1nnc(S(N)(=O)=O)s1 ; "or" ; *c1ccc2nc(S(N)(=O)=O)sc2c1]

and
R⁹ comprises a metal chelator (CL).
   It is understood that although formula (II) is shown without specific stereochemistry, the structure of formula (II) encompasses stereoisomers thereof, such as formula (IIa) and (IIb), as well as mixtures of stereoisomers thereof.

In some embodiments, q2 is 0. In some embodiments, q2 is 1.

Also disclosed herein is a compound of the following structural formula:

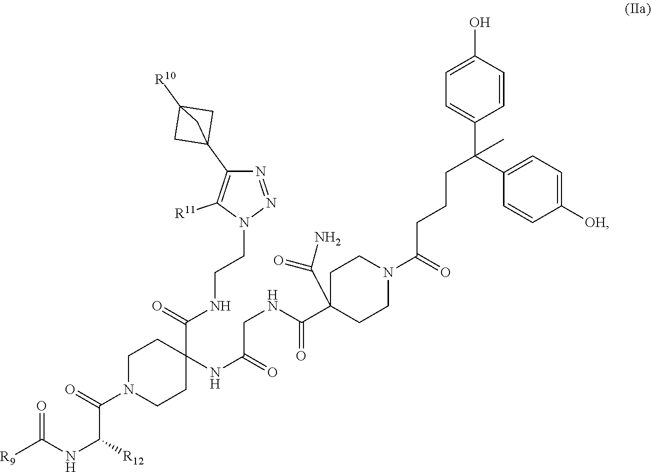

or a pharmaceutically acceptable salt thereof.

Also disclosed herein is a compound of the following structural formula:

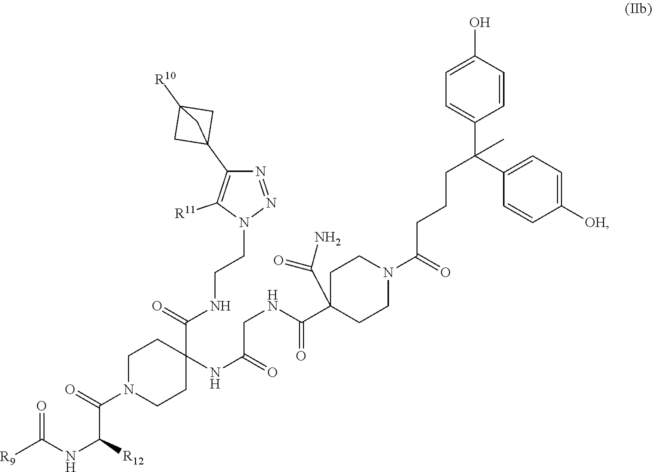

or a pharmaceutically acceptable salt thereof.

In some embodiments, R¹⁰ is

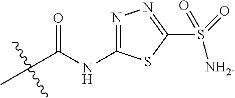 [SMILES: *C(=O)Nc1nnc(S(N)(=O)=O)s1]

In some embodiments, R¹⁰ is

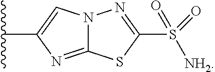 [SMILES: *c1cn2nc(S(N)(=O)=O)sc2n1]

In some embodiments, R¹⁰ is

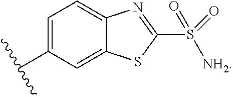 [SMILES: *c1ccc2nc(S(N)(=O)=O)sc2c1]

In some embodiments, wherein R⁹ is

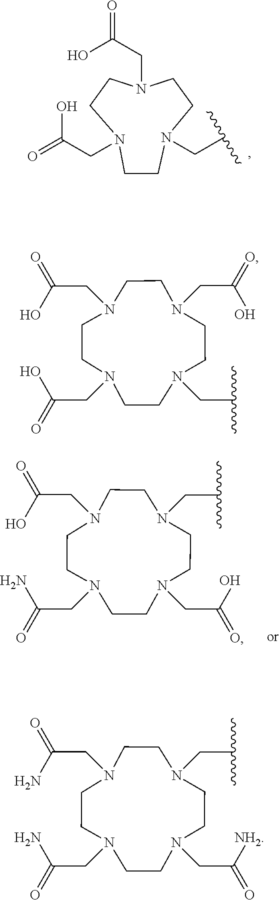

In some embodiments, R⁹ is

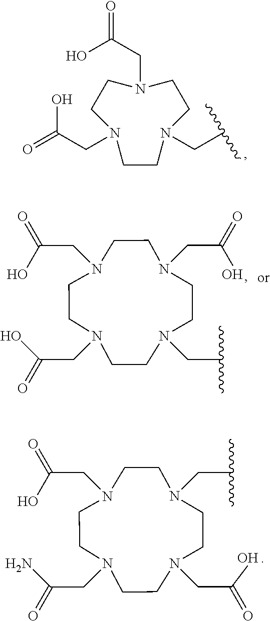 [SMILES: *CN1CCN(CC(=O)O)CCN(CC(=O)O)CC1 ; *CN1CCN(CC(=O)O)CCN(CC(=O)O)CCN(CC(=O)O)CC1 ; *CN1CCN(CC(=O)O)CCN(CC(N)=O)CCN(CC(=O)O)CC1]

In some embodiments, R⁹ is

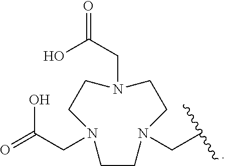 [SMILES: *CN1CCN(CC(=O)O)CCN(CC(=O)O)CC1]

In some embodiments, R⁹ is

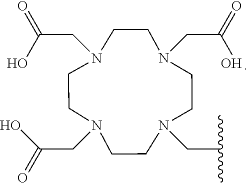 [SMILES: *CN1CCN(CC(=O)O)CCN(CC(=O)O)CCN(CC(=O)O)CC1]

In some embodiments, R⁹ is

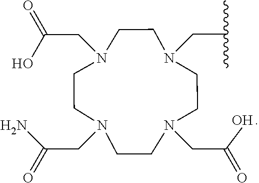 [SMILES: *CN1CCN(CC(=O)O)CCN(CC(N)=O)CCN(CC(=O)O)CC1]

In some embodiments, R⁹ is

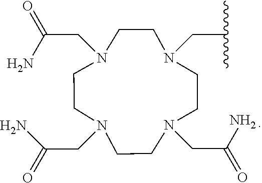 [SMILES: *CN1CCN(CC(N)=O)CCN(CC(N)=O)CCN(CC(N)=O)CC1]

In some embodiments, R¹¹ is H. In alternative embodiments, R¹¹ is —COOH.

In some embodiments, R¹² is

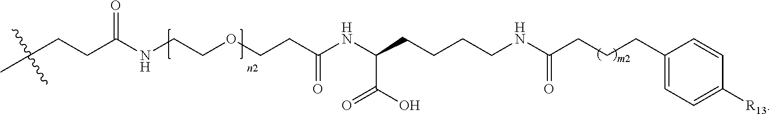

In some embodiments, R¹² is

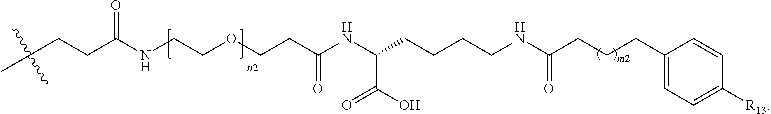

In some embodiments, n2 is 6, 8, or 16. In some embodiments, n2 is 6. In some embodiments, n2 is 8. In some embodiments, n2 is 16.

In some embodiments, m2 is 1. In some embodiments, m2 is 2. In some embodiments, m2 is 3.

In some embodiments, R₁₃ is H. In some embodiments, R₁₃ is halogen. In some embodiments, R₁₃ is F. In some embodiments, R₁₃ is Cl. In some embodiments, R₁₃ is Br. In some embodiments, R₁₃ is I. In some embodiments, R₁₃ is —CH₃. In some embodiments, R₁₃ is —OCH₃. In some embodiments, R₁₃ is —OCH₂CH₃.

Also disclosed herein is a compound of the following structure:

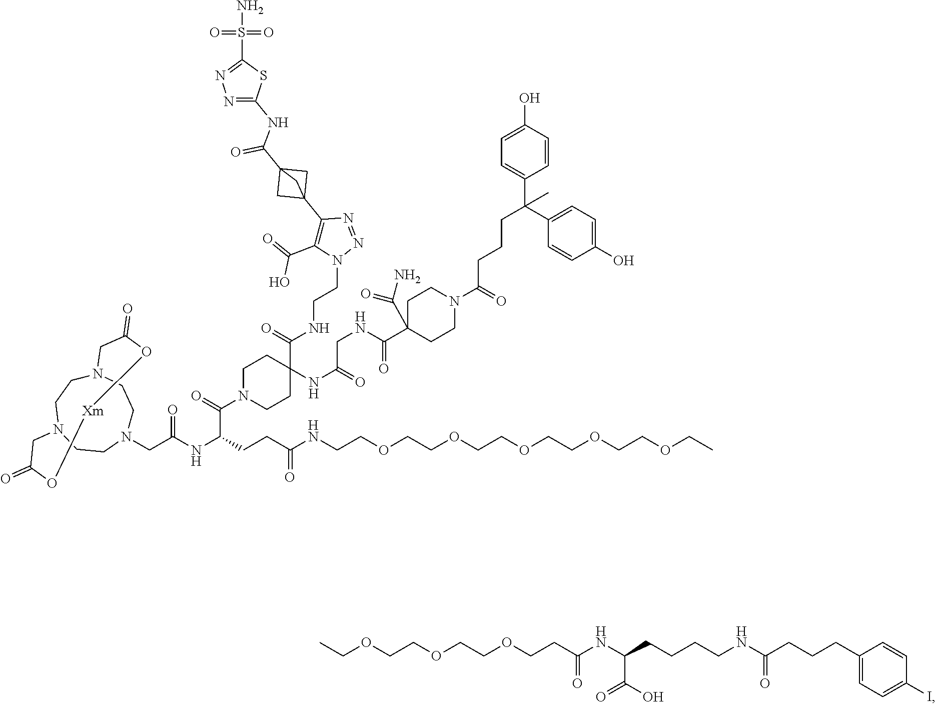

or a stereoisomer or mixture of stereoisomers, or pharmaceutically acceptable salt thereof, wherein Xₘ is a radionuclide.

Also disclosed herein is a compound of the following structure:

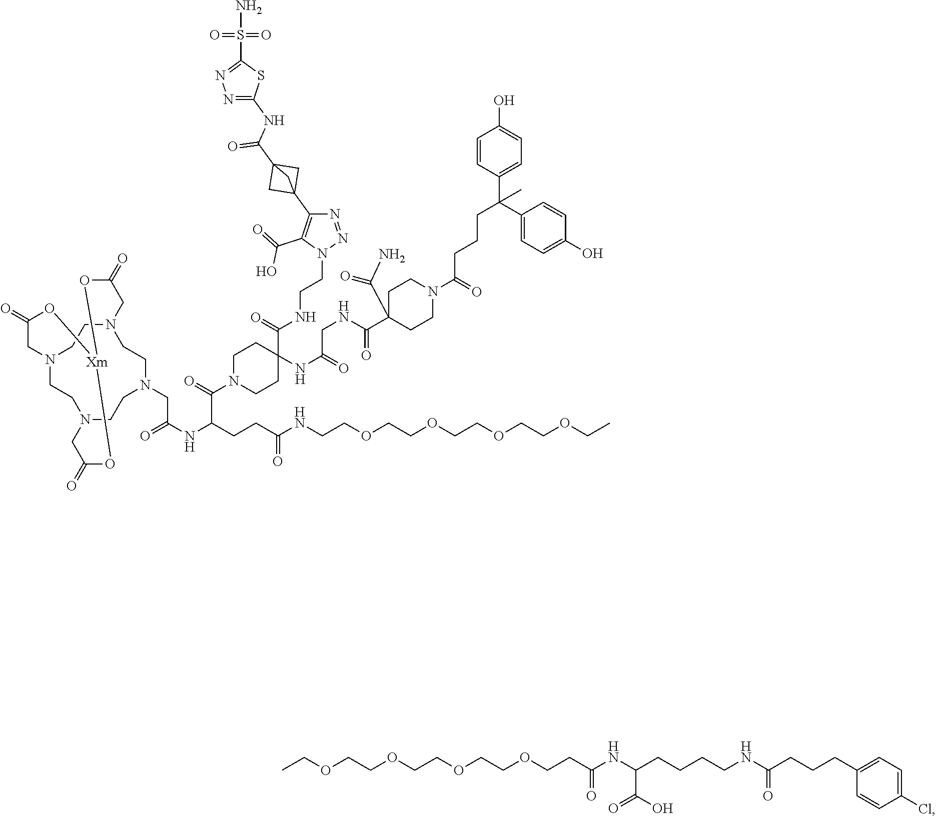

or a stereoisomer or mixture of stereoisomers, or pharmaceutically acceptable salt thereof, wherein Xₘ is a radionuclide.

Also disclosed herein is a compound of the following structure:

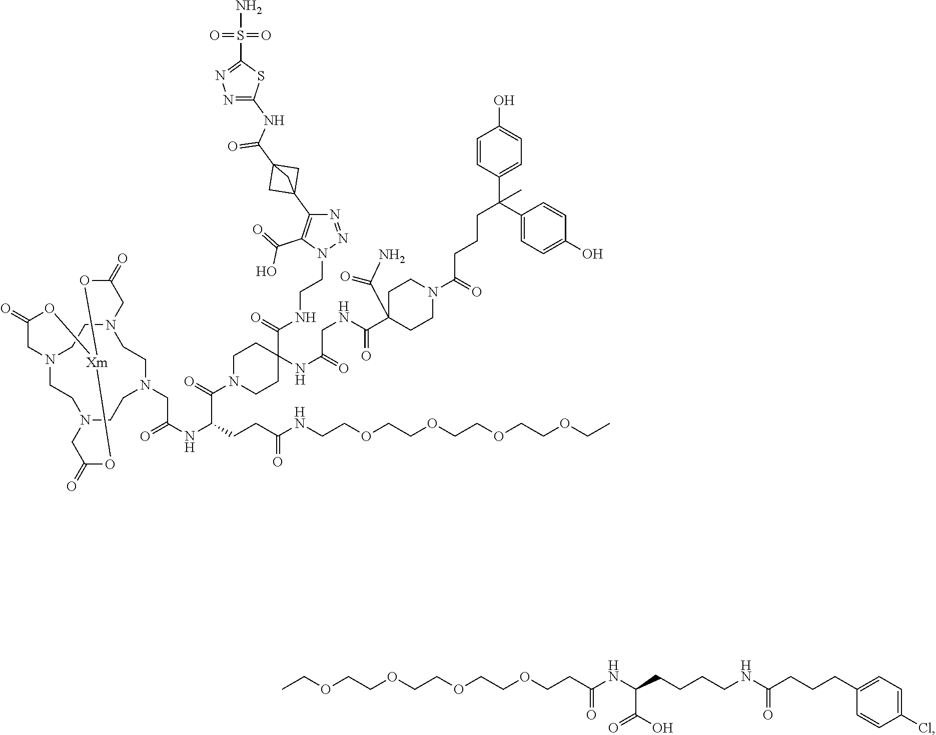

or a stereoisomer or mixture of stereoisomers, or pharmaceutically acceptable salt thereof, wherein Xₘ is a radionuclide.

Also disclosed herein is a compound of the following structure:

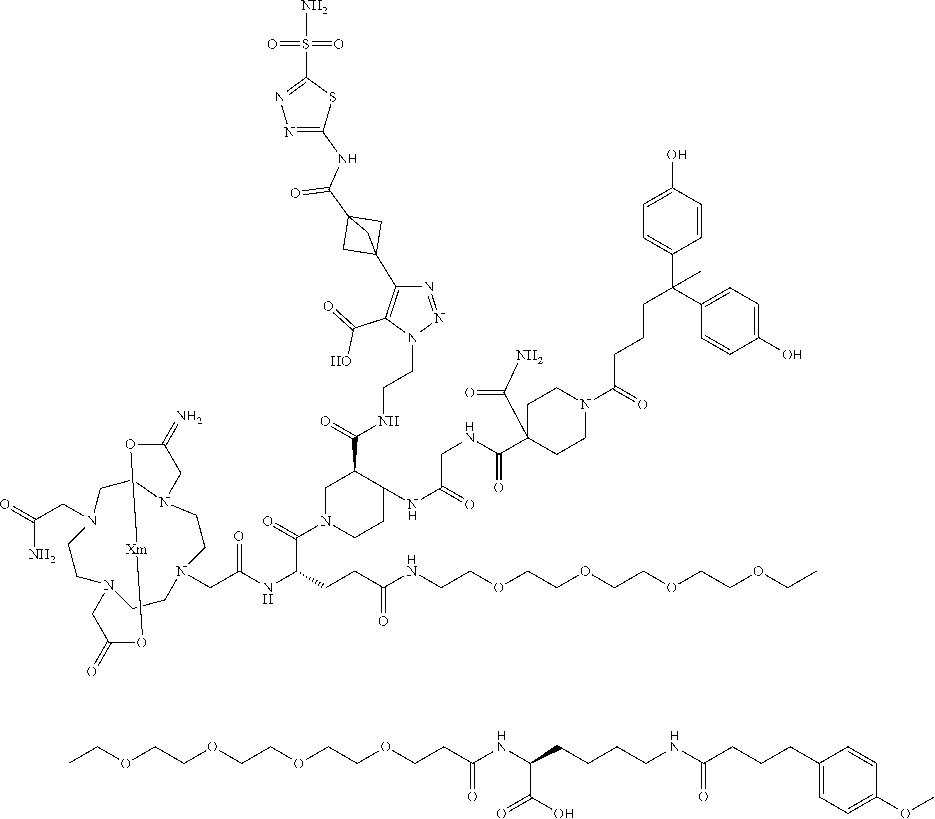

or a stereoisomer or mixture of stereoisomers, or pharmaceutically acceptable salt thereof, wherein Xₘ is a radionuclide.

A compound of the following structure:

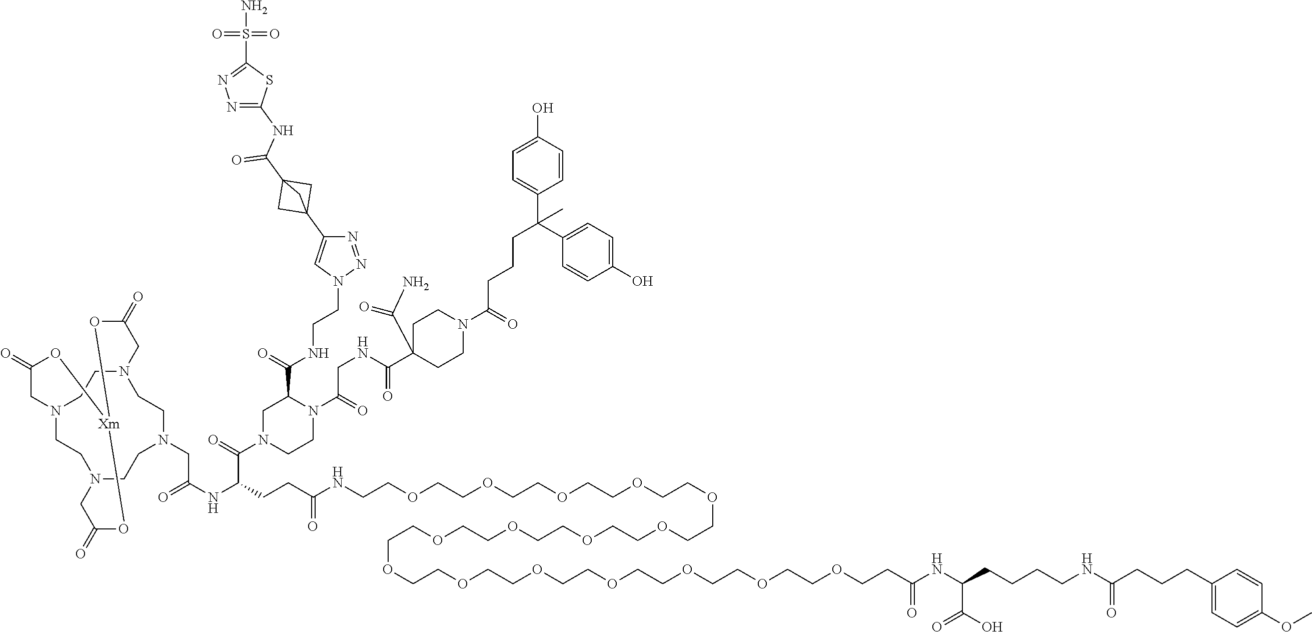

or a stereoisomer or mixture of stereoisomers, or pharmaceutically acceptable salt thereof, wherein Xₘ is a radionuclide.

In some embodiments of a compound of Formula (I), (Ia), (Ib), (II), (IIa), and/or (IIb) the metal chelator is a metal chelator disclosed herein. In some embodiments, the metal chelator (CL) is NOTA, DOTA, or DOTA-monoamide.

In some embodiments of a compound of Formula (I), (Ia), (Ib), (II), (IIa), and/or (IIb) the compound further comprises a radionuclide bound to the metal chelator (CL).

In some embodiments of any of the aforementioned structural formulas (e.g., a compound of Formula (I), (Ia), (Ib), (II), (IIa), and/or (IIb)), the radionuclide is an alpha particle-emitting radionuclide. In some embodiments, the alpha particle-emitting radionuclide is Ac-225, Bi-213, Bi-209, Tb-149, Ra-223, Th-227, Fr-223, Gd-148, Th-229, Pb-212, or Po-213. In some embodiments, the alpha particle-emitting radionuclide is Ac-225, Bi-212, Bi-213, Bi-209, Tb-149, Ra-223, Ra-224, Th-227, Fr-223, Gd-148, Th-229, Pb-212, or Po-213. Ac-225, or Pb-212. In some embodiments, the alpha particle-emitting radionuclide is Pb-212. In some embodiments, the alpha particle-emitting radionuclide is Ac-225. In some embodiments, the radionuclide is a beta particle-emitting radionuclide. In some embodiments, the beta particle-emitting radionuclide is Cu-67, Lu-177, Y-90, Rh-105, Yb-175, Tm-167, Pm-153, Sm-153, or In-111. In some embodiments, the beta particle-emitting radionuclide is Cu-67, Lu-177, Y-90, Rh-105, Yb-175, Tm-167, Pm-153, Sm-153, or Tb-161. In some embodiments, the beta particle-emitting radionuclide is Lu-177, Tb-161, or Cu-67. In some embodiments, the beta particle-emitting radionuclide is lutetium-177. In some embodiments, the photon-emitting radionuclide is Tc-99m, In-111, Ga-67, Y-90, Lu-177, Sm-153, Re-186, Re-188, or Tb-161. In some embodiments, the photon-emitting radionuclide is In-111, Ga-67, Lu-177, or Tb-161. In some embodiments, the photon-emitting radionuclide is In-111 or Tb-161. In some embodiments, the photon-emitting radionuclide is Tb-161. In some embodiments, the photon-emitting radionuclide is In-111. In some embodiments, the radionuclide is a positron-emitting radionuclide. In some embodiments, positron-emitting radionuclide is Ga-68, Cu-61, Cu-62, Cu-64, Zr-89, Tb-152, Sc-44, Y-86, Ti-45, Mn-52, As-72, or AlF-18. In some embodiments, the positron-emitting radionuclide is Ga-68, Cu-61, Cu-62, Cu-64, Tb-152, or AlF-18. In some embodiments, the positron-emitting radionuclide is Cu-61, Cu-62, or Cu-64. In some embodiments, the positron-emitting radionuclide is Ga-68. In some embodiments, the positron-emitting radionuclide is Tb-152. In some embodiments, the positron-emitting radionuclide is AlF-18. In some embodiments, the radionuclide is Cu-67, Lu-177, Ac-225, or Ga-68. In some embodiments, the radionuclide is Cu-67, Lu-177, Ac-225, In-111, or Tb-161, Ga-68.

Representative compounds of structural formulas (I), (Ia), (Ib), (II), (Ila), and (IIb) are set forth in Table 1 and Table 2. In some embodiments, provided herein is one or more compounds of Table 1, or Table 2, or a stereoisomer or mixture of stereoisomers, or pharmaceutically acceptable salt thereof. In some embodiments, provided herein is a compound of Table 1, or a stereoisomer or mixture of stereoisomers, or pharmaceutically acceptable salt thereof. In some embodiments, provided herein is a compound of Table 2, or a stereoisomer or mixture of stereoisomers, or pharmaceutically acceptable salt thereof. In some embodiments, provided herein is one or more compounds of Table 1, or Table 2, or a pharmaceutically acceptable salt thereof. In some embodiments, provided herein is a compound of Table 1, or a pharmaceutically acceptable salt thereof. In some embodiments, provided herein is a compound of Table 2, or a pharmaceutically acceptable salt thereof.

In the descriptions herein, it is understood that all descriptions, variations, embodiments, or aspects of formula (I) or formula (II), where applicable, apply equally to other formulae detailed herein, and are equally described, the same as if each and every description, variation, embodiment, or aspect were separately and individually listed. It is also understood that all descriptions, variations, embodiments, or aspects of formula (I) or formula (II), where applicable, apply equally to other formulae detailed herein, and are equally described, the same as if each and every description, variation, embodiment, or aspect were separately and individually listed for all formulae. For example, all descriptions, variations, embodiments, or aspects of formula (I) or formula (II), where applicable, apply equally to any of the formulae as detailed herein, such as formulae (la), (Ib), (Ila), and (IIb), and are equally described, the same as if each and every description, variation, embodiment, or aspect were separately and individually listed for all formulae.

### Metal Chelators

In one aspect, described herein are compounds that comprise a metal chelator, e.g., as represented by CL in the formulas described herein.

In some embodiments, the metal chelator binds with a radionuclide. In some embodiments, the compounds described herein comprise two or more independent metal chelators, e.g., 2, 3, 4, 5, or more metal chelators. In some embodiments, the compounds described herein comprise two metal chelators, which can be the same or different. In some embodiments, the compounds described herein comprise two or more metal chelators. In some embodiments, the compounds comprise two radionuclides bound to the metal chelators.

In some embodiments, the metal chelator is capable of binding a radioactive atom. The binding can be direct, e.g., the metal chelator can make hydrogen bonds or electrostatic interactions with the radioactive atom. The binding can also be indirect, e.g., the metal chelator binds to a molecule that comprises a radioactive atom. In some embodiments, the metal chelator comprises, or is, a macrocycle. In some embodiments, the metal chelator comprises, or is, 2,2′,2″,2″-(1,4,7,10-Tetraazacyclododecane-1,4,7,10-tetrayl)tetraacetic acid (DOTA) or 1,4,7-triazacyclononane-1,4,7-triacetic acid (NOTA). In some embodiments, the metal chelator comprises a macrocycle, e.g., a macrocycle comprising an oxygen atom and/or a nitrogen atom, DOTA, NOTA, one or more amines, one or more ethers, one or more carboxylic acids, EDTA, DTPA, TETA, DO3A, PCTA, or desferrioxamine.

In some embodiments, the metal chelator comprises a plurality of amines. In some embodiments, the metal chelator includes 4 or more nitrogen atoms, 4 or more carboxylic acid groups, or a combination thereof. In some embodiments, the metal chelator does not comprise S. In some embodiments, the metal chelator comprises a ring. In some embodiments, the ring comprises an oxygen atom and/or a nitrogen atom. In some embodiments, the metal chelator is a ring that includes 3 or more nitrogen atoms, 3 or more carboxylic acid groups, or a combination thereof. In some embodiments, the metal chelator is polydentate.

In some embodiments, the metal chelator (or CL) described herein is DOTA, DOTA-GA, pBn-DOTA, pBn-SCN-DOTA, NH2-DOTA, NH2-DOTA-GA, p-NCS-Bn-DOTA-GA, p-NH2-Bn-oxo-DO3A, p-SCN-Bn-oxo-DO3A, NOTA, NODA-GA, NH2-NODA-GA, p-NCS-Bn-NODA-GA, p-NH2-Bn-NOTA, p-SCN-Bn-NOTA, NCS-MP-NODA, NH2-MPAA-NODA, PCTA, p-NH2-Bn-PCTA, p-SCN-Bn-PCTA, p-SCN-Bn-HEHA, H2-MACROPA-NCS, H1-MACROPA, H2-MACROPA-NH2, H4-OCTAPA, tetra-(S, S, S, S)-Me-DOTA, tetra-(S, S, S, S)-Et-DOTA, tetra-(S, S, S, S)-iBu-DOTA, maleimide-nBu-DOTA, or DOTA-monoamide. In some embodiments, the metal chelator is NOTA, DOTA, or DOTA-monoamide.

In some embodiments, the metal chelator (or CL) described herein comprises a cyclic chelating agent. Exemplary cyclic chelating agents include, but are not limited to, AAZTA, BAT, BAT-TM, Crown, Cyclen, DO2A, CB-DO2A, DO3A, H3HP-DO3A, Oxo-DO3A, p-NH2-Bn-Oxo-DO3A, DOTA, DOTA-3py, DOTA-PA, DOTA-GA, DOTA-4AMP, DOTA-2py, DOTA-1py, p-SCN-Bn-DOTA, CHX-A″-EDTA, MeO-DOTA-NCS EDTA, DOTAMAP, DOTAGA, DOTAGA-anhydride, DOTMA, DOTASA, DOTAM, DOTP, CB-Cyclam, TE2A, CB-TE2A, CB-TE2P, DM-TE2A, MM-TE2A, NOTA, NOTP, HEHA, HEHA-NCS, p-SCN-Bn-HEHA, DTPA, CHX-A″-DTPA, p-NH2-Bn-CHX-A″-DTPA, p-SCN-DTPA, p-SCN-Bz-Mx-DTPA, 1B4M-DTPA, p-SCN-Bn1B-DTPA, p-SCN-Bn-1B4M-DTPA, p-SCN-Bn-CHX-A″-DTPA, PEPA, p-SCN-Bn-PEPA, TETPA, DOTPA, DOTMP, DOTPM, t-Bu-calix[4] arene-tetracarboxylic acid, macropa, macropa-NCS, macropid, H3L1, H3L4, H2azapa, H5decapa, bispa2, H4pypa, H4octapa, H4CHXoctapa, p-SCN-Bn-H4octapa, p-SCN-Bn-H4octapa, TTHA, p-NO2-Bn-neunpa, H4octox, H2macropa, H2bispa2, H4phospa, H6phospa, p-SCN-Bn-H6phospa, TETA, p-NO2-Bn-TETA, TRAP, TPA, HBED, SHBED, HBED-CC, (HBED-CC) TFP, DMSA, DMPS, DHLA, lipoic acid, TGA, BAL, Bis-thioseminarabazones, p-SCN-NOTA, nNOTA, NODAGA, CB-TEIAIP, 3P-C-NETA-NCS, 3p-C-DEPA, 3P-C-DEPA-NCS, TCMC, PCTA, NODIA-Me, TACN, pycup1A1B, pycup2A, THP, DEDPA, H2DEDPA, p-SCN-Bn-H2DEDPA, p-SCN-Bn-TCMC, motexafin, NTA, NOC, 3p-C-NETA, p-NH2-Bn-TE3A, SarAr, DiAmSar, SarAr-NCS, AmBaSar, BaBaSar, TACN-TM, CP256, C-NE3TA, C-NE3TA-NCS, NODASA, NETA-monoamide, C-NETA, NOPO, BPCA, p-SCN-Bn-DFO, DFO-ChX-Mal, DFO, DFO-IAC, DFO-BAC, DIP-LICAM, EC, SBAD, BAPEN, TACHPYR, NEC-SP, Lpy, L1, L2, L3, and EuK-106. In some embodiments, the metal chelator is DOTA, TRITA, TETA, DOTA-MA, DO3A-HP, DOTMA, DOTA-pNB, DOTP, DOTMP, DOTEP, DOTMPE, F-DOTPME, DOTPP, DOTBzP, DOTA-monoamide, p-NCS-DOTA, p-NCS-PADOTA, BAT, DO3TMP-Monoamide, p-NCS-TRITA, NOTA, or CHX-A″-DTPA.

In some embodiments, the metal chelator described herein comprises an acyclic chelating agent. Exemplary acyclic chelating agents include, but are not limited to, DTA, CyEDTA, EDTMP, DTPMP, DTPA, CyDTPA, Cy2DTPA, DTPA-MA, DTPA-BA, and BOPA. In some embodiments, the metal chelator described herein comprises DOTA, DOTP, DOTMA, DOTAM, DTPA, NTA, EDTA, DO3A, DO2A, NOC, NOTA, TETA, TACN, DiAmSar, CB-Cyclam, CB-TE2A, DOTA-4AMP, or NOTP. In some embodiments, the metal chelator described herein comprises H4pypa, H4octox, H4octapa, p-NO2-Bn-neunpa, p-SCN-Bn-H4neunpa, TTHA, tBu4pypa-C7-NHS, H4neunpa, H2macropa, HP-DO3A, BT-DO3A, DO3A-Nprop, DO3AP, DO2A2P, DOA3P, DOTP, DOTPMB, DOTAMAE, DOTAMAP, DO3AMBu, DOTMA, TCE-DOTA, DEPA, PCTA, p-NO2-Bn-PCTA, p-NO2-Bn-DOTA, symPC2APA, symPCA2PA, asymPC2APA, asymPCA2PA, TRAP, AAZTA, DATAm, THP, HEHA, or HBED.

In some embodiments, the metal chelator (or CL) is DO3A. In some embodiments, the metal chelator is PEPA. In some embodiments, the metal chelator is EDTA. In some embodiments, the metal chelator is CHX-A″-DTPA. In some embodiments, the metal chelator is HEHA. In some embodiments, the metal chelator is DOTMP. In some embodiments, the metal chelator is t-Bu-calix[4] arene-tetracarboxylic acid. In some embodiments, the metal chelator is macropa. In some embodiments, the metal chelator is macropa-NCS. In some embodiments, the metal chelator is H4pypa. In some embodiments, the metal chelator is H4octapa. In some embodiments, the metal chelator is H4CHXoctapa. In some embodiments, the metal chelator is DOTP. In some embodiments, the metal chelator is a crown.

Exemplary metal chelators are further described in WO2012/174136; US20130183235A1; US20120219495A1; Ramogidaand et al., EJNMMI radiopharm. chem. 4, 21 (2019); Thiele et al., Cancer Biotherapy and Radiopharmaceuticals 2018; Li et al., Bioconjugate Chem. 2019, 30, 5, 1539-1553; and Baranyai et al., Eur. J. Inorg. Chem. 36-56 (2020), Dai et al., Nature Communications (2018) 9:857, each of which is incorporated by reference in its entirety.

In some embodiments, the metal chelator (or CL) is DOTA. In some embodiments, the metal chelator is a chiral derivative of DOTA. In some embodiments, the metal chelator is 2,2′,2″,2′-((2S,5S,8S,11S)-2,5,8,11-tetramethyl-1,4,7,10-tetraazacyclododecane-1,4,7,10-tetrayl)tetraacetic acid.

In some embodiments, the metal chelator (or CL) is

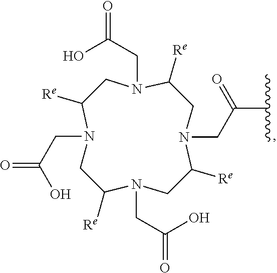

wherein each R^{e} is independently selected from hydrogen, alkyl, haloalkyl, hydroxyalkyl, aminoalkyl, heteroalkyl, alkenyl, alkynyl, cycloalkyl, heterocycloalkyl, aryl, heteroaryl, alkylcycloalkyl, alkylheterocycloalkyl, alkylaryl, alkylheteroaryl, or an amino acid side chain. In some embodiments, the metal chelator is

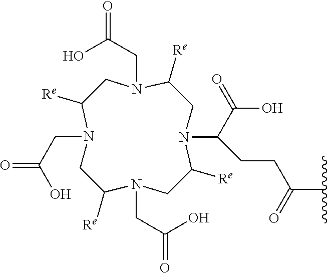

wherein each R^{e} is independently selected from hydrogen, alkyl, haloalkyl, hydroxyalkyl, aminoalkyl, heteroalkyl, alkenyl, alkynyl, cycloalkyl, heterocycloalkyl, aryl, heteroaryl, alkylcycloalkyl, alkylheterocycloalkyl, alkylaryl, alkylheteroaryl, or an amino acid side chain.

In some embodiments, the metal chelator (or CL) is

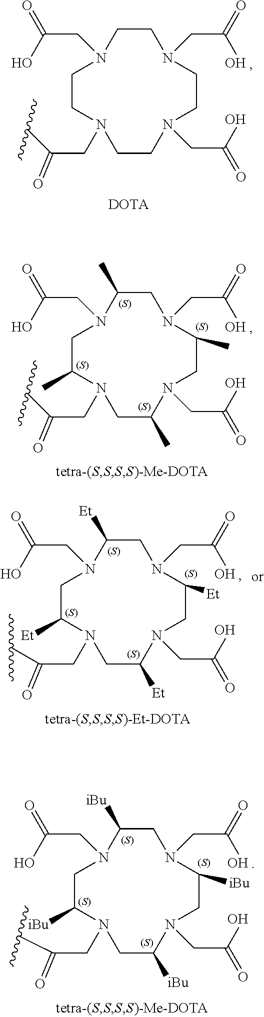

In some embodiments, the metal chelator is

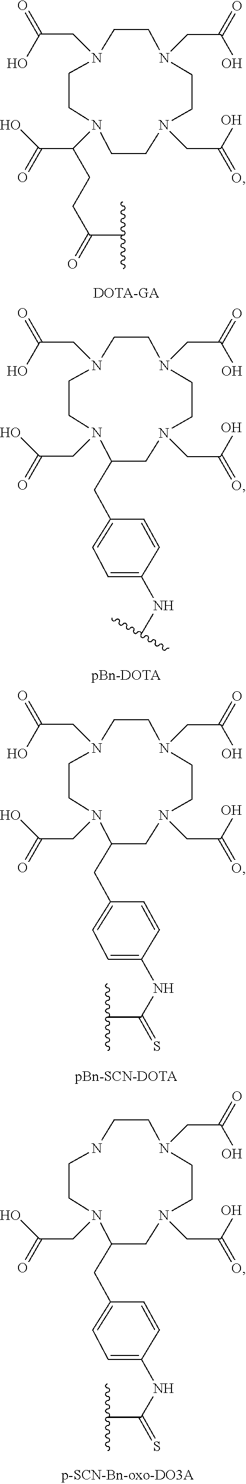 [SMILES: *C(=O)CCC(C(=O)O)N1CCN(CC(=O)O)CCN(CC(=O)O)CCN(CC(=O)O)CC1 ; *Nc1ccc(CC2CN(CC(=O)O)CCN(CC(=O)O)CCN(CC(=O)O)CCN2CC(=O)O)cc1 ; *C(=S)Nc1ccc(CC2CN(CC(=O)O)CCN(CC(=O)O)CCN(CC(=O)O)CCN2CC(=O)O)cc1 ; *C(=S)Nc1ccc(CC2CN(CC(=O)O)CCN(CC(=O)O)CCNCCN2CC(=O)O)cc1]

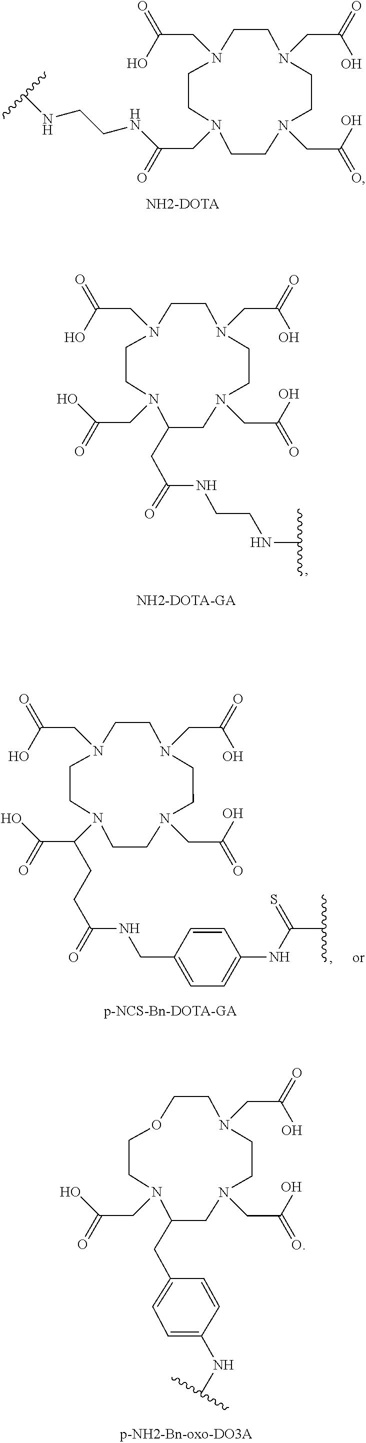

In some embodiments, the metal chelator is

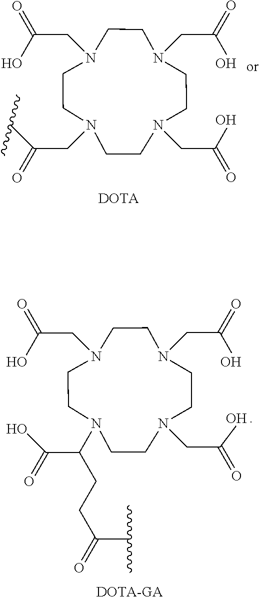

In some embodiments, the metal chelator is

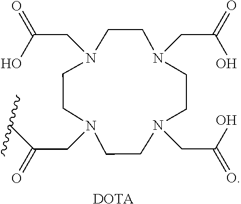

In some embodiments, the metal chelator is

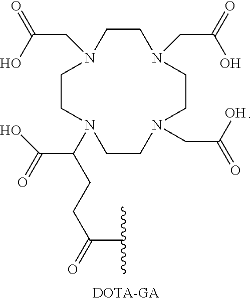 [SMILES: *C(=O)CCC(C(=O)O)N1CCN(CC(=O)O)CCN(CC(=O)O)CCN(CC(=O)O)CC1]

In some embodiments, the metal chelator is

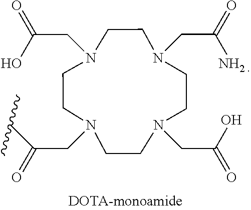 [SMILES: *C(=O)CN1CCN(CC(=O)O)CCN(CC(N)=O)CCN(CC(=O)O)CC1]

In some embodiments, the metal chelator is

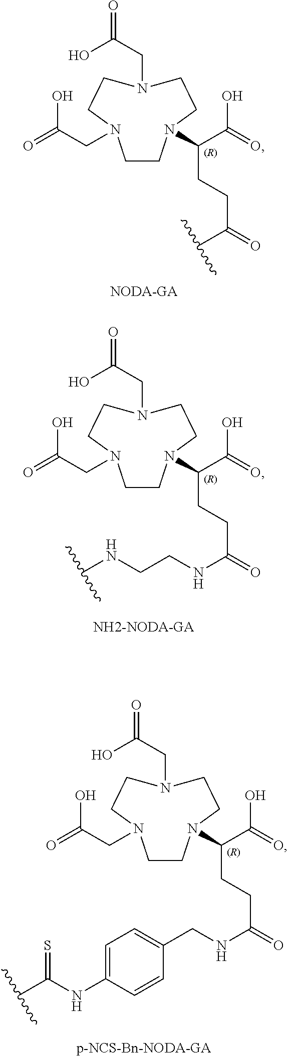

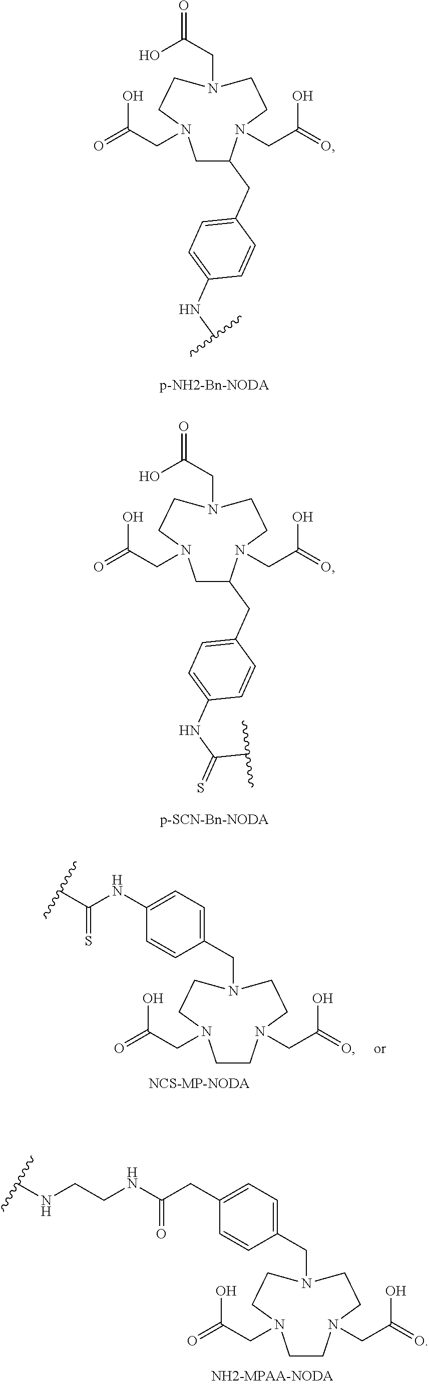 [SMILES: *Nc1ccc(CC2CN(CC(=O)O)CCN(CC(=O)O)CCN2CC(=O)O)cc1 ; *C(=S)Nc1ccc(CC2CN(CC(=O)O)CCN(CC(=O)O)CCN2CC(=O)O)cc1 ; *C(=S)Nc1ccc(CN2CCN(CC(=O)O)CCN(CC(=O)O)CC2)cc1 ; *NCCNC(=O)Cc1ccc(CN2CCN(CC(=O)O)CCN(CC(=O)O)CC2)cc1]

In some embodiments, the metal chelator is

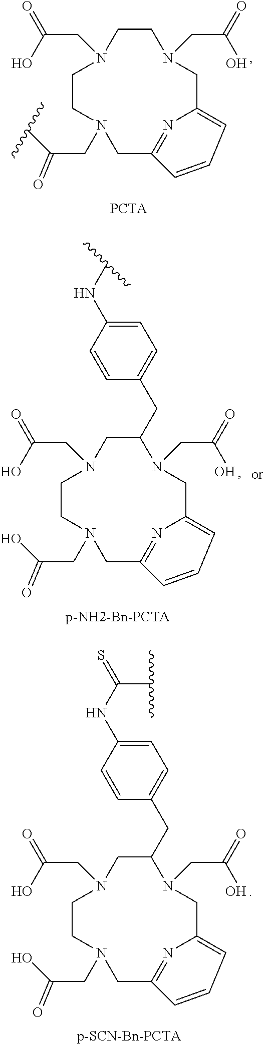 [SMILES: *C(=O)CN1CCN(CC(=O)O)CCN(CC(=O)O)Cc2cccc(n2)C1 ; *Nc1ccc(CC2CN(CC(=O)O)CCN(CC(=O)O)Cc3cccc(n3)CN2CC(=O)O)cc1 ; *C(=S)Nc1ccc(CC2CN(CC(=O)O)CCN(CC(=O)O)Cc3cccc(n3)CN2CC(=O)O)cc1]

In some embodiments, the metal chelator (or CL) is

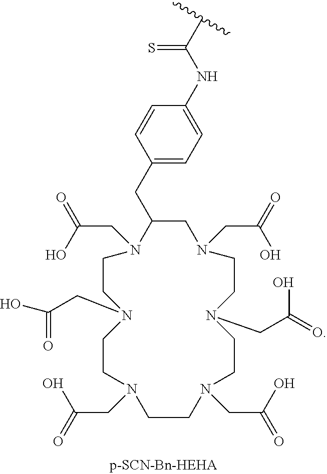 [SMILES: *C(=S)Nc1ccc(CC2CN(CC(=O)O)CCN(CC(=O)O)CCN(CC(=O)O)CCN(CC(=O)O)CCN(CC(=O)O)CCN2CC(=O)O)cc1]

In some embodiments, the metal chelator is

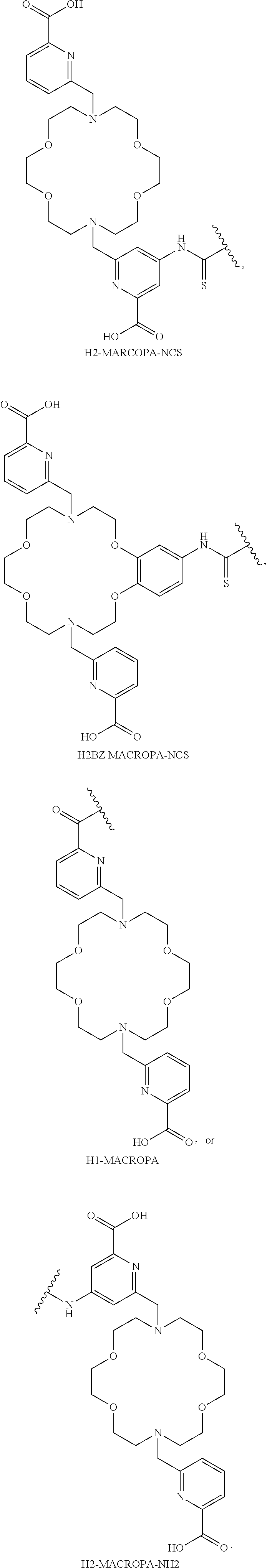

In some embodiments, the metal chelator is NOTA, DOTA, DOTA-monoamide, or DOTAM.

In some embodiments, the metal chelator is a metal chelator illustrated in any of FIGS. 1 to 19.

### Radiopharmaceutical Compounds

Disclosed herein are compounds disclosed herein bound to a radionuclide, e.g., compounds comprising a radionuclide bound to a metal chelator (CL) of the compound.

Generally, the type of radionuclide used in a therapeutic radiopharmaceutical can be tailored to the specific type of cancer, the type of targeting moiety (e.g., binding peptide), etc. Radionuclides that undergo a-decay produce particles composed of two neutrons and two protons, and radionuclides that undergo B-decay emit energetic electrons from their nuclei. Some radionuclides can also undergo electron capture and emit Auger electrons. In some embodiments, the compounds comprise an alpha particle-emitting radionuclide. Alpha radiation can cause direct, irreparable double-strand DNA breaks compared with gamma and beta radiation, which can cause single-stranded breaks via indirect DNA damage. The range of these particles in tissue and the half-life of the radionuclide can also be considered in designing the radiopharmaceutical compounds. Table A below illustrates some properties of exemplary radionuclides.

TABLE A
Exemplary radionuclides

| | | Therapeutics | | | Therapeutics |
|---|---|---|---|---|---|
| | | (Tx) or | | | (Tx) or |
| | Emission/ | Diagnostics | | Emission/ | Diagnostics |
| Radionuclide | Decay | (Dx) | Radionuclide | Decay | (Dx) |
| Ac-225 | Alpha | Tx | I-131 | Beta | Tx |
| As-70 | Positron | Dx | In-111 | Photon/Electron | Dx |
| | | | | Capture | |
| As-71 | Positron | Dx | Lu-177 | Beta/Photon | Tx/Dx |
| As-72 | Positron | Dx | Mn-52 | Positron | Dx |
| As-73 | Electron Capture | Dx | Pb-212 | Alpha | Tx |
| As-74 | Positron | Dx | Pm-153 | Beta | Tx |
| As-76 | Beta | Tx | Po-213 | Alpha | Tx |
| As-77 | Beta | Tx | Ra-223 | Alpha | Tx |
| At-211 | Alpha | Tx | Ra-224 | Alpha | Tx |
| Bi-209 | Alpha | Tx | Re-186 | Beta/Photon | Dx |
| Bi-212 | Alpha | Tx | Re-188 | Beta/Photon | Dx |
| Bi-213 | Alpha | Tx | Rh-105 | Beta | Tx |
| Br-76 | Positron | Dx | Sm-153 | Beta/Photon | Tx/Dx |
| Ce-134 | Electron Capture | Dx | Sc-44 | Beta/Positron | Dx |
| Cu-61 | Positron | Dx | Tb-149 | Alpha | Tx |
| Cu-62 | Positron | Dx | Tb-152 | Positron | Dx |
| Cu-64 | Positron | Dx | Tb-161 | Beta/Photon | Tx/Dx |
| Cu-67 | Beta | Tx | Tc-99m | Photon/Gamma/ | Dx |
| | | | | Isomeric Transition | |
| F-18 | Positron | Dx | Th-227 | Alpha | Tx |
| Fr-223 | Alpha | Tx | Th-229 | Alpha | Tx |
| Ga-67 | Beta/Photon | Dx | Ti-45 | Positron | Dx |
| Ga-68 | Positron | Dx | Tm-167 | Beta/Electron | Tx |
| | | | | Capture | |
| Gd-148 | Alpha | Tx | Y-86 | Positron | Dx |
| Ho-166 | Beta/gamma | Tx/Dx | Y-90 | Beta/Photon | Tx/Dx |
| I-123 | Electron Capture/ | Dx | Yb-175 | Beta | Tx |
| | photon | | | | |
| I-124 | Positron | Dx | Zr-89 | Positron | Dx |
| I-125 | Electron Capture | Dx | | | |

In some embodiments, compounds described herein comprise a radionuclide selected from Table A. In some embodiments, disclosed herein are compounds of Table 1 or Table 2, or a salt (such as a pharmaceutically acceptable salt) or stereoisomer thereof, complexed with a radionuclide selected from Table A. In some embodiments, disclosed herein are compositions comprising a compound of Table 1 or Table 2, or a salt (such as a pharmaceutically acceptable salt) or stereoisomer thereof, complexed with a radionuclide selected from Table A. In some embodiments of any of the foregoing, the radionuclide is diagnostic. In some embodiments of any of the foregoing, the radionuclide is therapeutic.

In some embodiments, the compounds described herein comprise an alpha particle-emitting radionuclide. In some embodiments, the compound comprises an alpha-particle emitting radionuclide bound to the metal chelator. In some embodiments, the alpha particle-emitting radionuclide is actinium-225 (²²⁵Ac), radium-223 (²²³Ra), radium-224 (²²⁴Ra), bismuth-209 (²⁰⁹Bi), bismuth-213 (²¹³Bi), gadolinium-148 (¹⁴⁸Gd), terbium-149 (¹⁴⁹Tb), polonium-213 (²¹³Po), francium-223 (²²³Fr), thorium-227 (²²⁷Th), thorium-229 (²²⁹Th), or lead-212 (²¹²Pb). In some embodiments, the alpha particle-emitting radionuclide is selected from ²²⁵Ac, ²²³Ra, ²⁰⁹Bi, ²¹³Bi, ¹⁴⁸Gd, ¹⁴⁹Th, ²¹³Po, ²²³Fr, ²²⁷Th, ²²⁹Th, and ²¹²Pb. In some embodiments, the alpha particle-emitting radionuclide is Ac-225, Bi-212, Bi-213, Bi-209, Tb-149, Ra-223, Ra-224, Th-227, Fr-223, Gd-148, Th-229, Pb-212, or Po-213. In some embodiments, the alpha particle-emitting radionuclide is Ac-225 or Pb-212. In some embodiments, the alpha particle-emitting radionuclide is ²²⁵Ac. In some embodiments, the alpha particle-emitting radionuclide is ²¹³Bi. In some embodiments, the alpha particle-emitting radionuclide is ²¹²Bi. In some embodiments, the alpha particle-emitting radionuclide is ²¹²Pb. In some embodiments, the alpha particle-emitting radionuclide is ²²⁴Ra. In some embodiments, the alpha particle-emitting radionuclide is ²²³Ra. In some embodiments, the alpha particle-emitting radionuclide is ²²⁷Th. In some embodiments, the alpha particle-emitting radionuclide is ¹⁴⁹Tb.

In some embodiments, the compounds described herein comprise a radionuclide selected from ⁶²Cu, ⁶⁴Cu, ⁶⁷Cu, ⁹⁰Y, ¹⁰⁹Pd, ¹¹¹ Ag, ¹³⁴Ce, ¹⁴⁹Pm, ¹⁵³Sm, ¹⁶⁶Ho, ^{99m}Tc, ⁶⁷Ga, ⁶⁸Ga, ¹¹¹In, ⁹⁰Y, ¹⁷⁷Lu, ¹⁸⁶Re, ¹⁸⁸Re, ¹⁹⁷ Au, ¹⁹⁸ Au, ¹⁹⁹ Au, ¹⁰⁵Rh, ¹⁶⁵Ho, ¹⁶¹Tb, ¹⁴⁹Pm, ¹⁵³Pm, ⁴⁴Sc, ⁴⁷Sc, ²¹³Po, ²¹²Pb, ²⁰⁹Bi, ²¹²Bi, ²¹³Bi, ²²⁵Ac, ^{117m}Sn, ⁶⁷Ga, ¹⁴⁹Tb, ¹⁵²Tb, ¹⁶⁷Tm, ¹⁷⁵Yb, ²²³Ra, ²²³Fr, ²²⁷Th, ²²⁹Th, ²⁰¹Tl, ¹⁴⁸Gd, ¹⁶⁰Gd, ¹⁴⁸Nd, ⁸⁹Sr, and ⁸⁹Zr. In some embodiments, the radionuclide is selected from ⁶²Cu, ⁶⁴Cu, ⁶⁷Cu, ⁶⁸Ga, ⁸⁹Zr, ⁹⁰Y, ^{99m}Tc, ¹⁰⁵Rh, ¹¹¹In, ¹³⁴Ce, ¹⁴⁸Gd, ¹⁴⁹Tb, ¹⁵²Tb, ¹⁵³Pm, ¹⁶⁷Tm, ¹⁷⁵Yb, ¹⁷⁷Lu, ²⁰⁹Bi, ²¹²Pb, ²¹³Po, ²¹³Bi, ²²³Ra, ²²³Fr, ²²⁷Th, ²²⁵Ac, and ²²⁹Th. In some embodiments, the radionuclide is Cu-67, Lu-177, Ac-225, In-111, Tb-161, or Ga-68. In some embodiments, the radionuclide is ²²⁵Ac. In some embodiments, the radionuclide is a decay daughter of ²²⁵Ac such as ²²¹Fr, ²¹⁷At, ²¹³Bi, ²¹³Po, ²⁰⁹Tl, ²⁰⁹Pb, or ²⁰⁹Bi. In some embodiments, the compound comprises two ²²⁵Ac radionuclides. In some embodiments, the radionuclide is ¹⁷⁷Lu. In some embodiments, the compound comprises two ¹⁷⁷Lu radionuclides. In some embodiments, the radionuclide is no-carrier added (i.e., non-carrier-added or n.c.a.) ¹⁷⁷Lu. In some embodiments, the radionuclide is no-carrier added (i.e., non-carrier-added or n.c.a.) ²²⁵Ac. In some embodiments, the radionuclide is ¹⁷⁷Lu free of long-lived radioactive contaminants and byproducts. In some embodiments, the radionuclide is a non-carrier-added radionuclide. In some embodiments, the radionuclide is a pseudo-radiometal. In some embodiments, the pseudo-radiometal is aluminum-[¹⁸F]fluoride ([¹⁸F]AlF) complex.

In some embodiments, the compounds described herein comprise a radionuclide covalently attached to the compound. In some embodiments, disclosed herein are compounds of Table 1 or Table 2, or a salt (such as a pharmaceutically acceptable salt) or stereoisomer thereof, covalently attached to a radionuclide selected from At-211, F-18, I-123, or I-124. In certain embodiments, the nuclides are covalently attached to the compounds disclosed herein through click chemistry. Examples of click chemistry covalent conjugation of certain nuclides may be found in Kettenbach K, Schieferstein H, Ross TL. 18F-labeling using click cycloadditions. Biomed Res Int. 2014:361329, the disclosure of which is hereby incorporated by reference in its entirety.

In some embodiments, the compounds described herein comprise a beta particle-emitting radionuclide. In some embodiments, the compounds comprise a beta particle-emitting radionuclide bound to the metal chelator. In some embodiments, the beta particle-emitting radionuclide is copper-67, rhodium-105, ytterbium-175, thulium-167, promethium-153, yttrium-90, samarium-153, or lutetium-177. In some embodiments, the beta particle emitting radionuclide is copper-67, yttrium-90, samarium-153, or lutetium-177. In some embodiments, the beta particle emitting radionuclide is lutetium-177. In some embodiments, the beta particle-emitting radionuclide is Cu-67, Lu-177, Y-90, Rh-105, Yb-175, Tm-167, Pm-153, Sm-153, or Tb-161. In some embodiments, the beta particle-emitting radionuclide is Lu-177, Tb-161, or Cu-67. In some embodiments, the beta particle emitting radionuclide is Lu-177, Tb-161, or Cu-67.

In some embodiments, the compounds described herein comprise a gamma particle-emitting radionuclide. In some embodiments, the compound comprises a gamma particle-emitting radionuclide bound to the metal chelator. In some embodiments, the gamma particle-emitting radionuclide is indium-111 or tin-117m.

In some embodiments, the compounds described herein comprise a positron particle-emitting radionuclide. In some embodiments, the compounds comprise a positron particle-emitting radionuclide bound to the metal chelator. In some embodiments, the positron-emitting radionuclide is gallium-68, copper-61, copper-62, copper-64, zirconium-89, or terbium-152. In some embodiments, the radionuclide is zirconium-89. In some embodiments, the radionuclide is gallium-68. In some embodiments, the positron-emitting radionuclide is Ga-68, Cu-61, Cu-62, Cu-64, Zr-89, Tb-152, Sc-44, Y-86, Ti-45, Mn-52, As-72, or AlF-18. In some embodiments, the positron-emitting radionuclide is Ga-68, Cu-61, Cu-62, Cu-64, Tb-152, or AlF-18. In some embodiments, the positron-emitting radionuclide is Cu-61, Cu-62, or Cu-64. In some embodiments, the radionuclide is Tb-152. In some embodiments, the radionuclide is AlF-18.

In some embodiments, the compound described herein comprises a photon-emitting radionuclide. In some embodiments, the compounds comprise a photon-emitting radionuclide bound to the metal chelator. In some embodiments, the photon-emitting radionuclide is Tc-99m, In-111, Ga-67, Y-90, Lu-177, Sm-153, Re-186, Re-188, or Tb-161. In some embodiments, the photon-emitting radionuclide is In-111, Ga-67, Lu-177, or Tb-161. In some embodiments, the photon-emitting radionuclide is In-111 or Tb-161. In some embodiments, the photon-emitting radionuclide is Tb-161. In some embodiments, the photon-emitting radionuclide is In-111.

In some embodiments, the compound described herein comprises a radionuclide suitable for imaging or diagnostic purposes. In some embodiments, the radionuclide suitable for imaging is selected from ⁶²Cu, ⁶⁴Cu, ⁸⁹Zr, ¹³⁴Ce, ¹⁵²Tb, ⁶⁸Ga, ¹¹¹In, and ^{99m}Tc. In some embodiments, the radionuclide is suitable PET imaging. In some embodiments, the radionuclide suitable for PET imaging is selected from ⁶²Cu, ⁶⁴Cu, ⁸⁹Zr, ¹³⁴Ce, ¹⁵²Tb, and ⁶⁸Ga. In some embodiments, the radionuclide is suitable for SPECT imaging. In some embodiments, the radionuclide suitable for SPECT imaging is selected from ¹¹¹In and ^{99m}Tc.

In some embodiments, the radionuclide is Cu-67, Lu-177, Ac-225, or Ga-68. In some embodiments, the radionuclide is Cu-67, Lu-177, Ac-225, In-111, Tb-161, or Ga-68.

In some embodiments, the compounds described herein do not contain any hot radionuclide, i.e., a cold conjugate. For example, in some cases, a radionuclide can be replaced with a surrogate (e.g., ²²⁵Ac replaced with lanthanum) for testing and experimental purposes. In some embodiments, hot lutetium (Lu-177) is replaced with a cold Lutetium (Lu-175).

A metal chelator such as DOTA can interact with a radionuclide (e.g., ¹⁷⁷Lu or ²²⁵Ac) via one or more functional groups and/or atoms. For example, the metal chelator can interact with a radionuclide via a nitrogen and/or an oxygen atom. As another example, the metal chelator can interact with a radionuclide via carbonyl, carboxylic acid, amino, and/or amide groups of the metal chelator. In some embodiments, the interaction of the metal chelator and a radionuclide of the compounds disclosed herein can be illustrated as

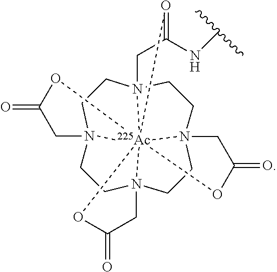

In some embodiments, the interaction of the metal chelator and a radionuclide of the compounds disclosed herein can be illustrated as

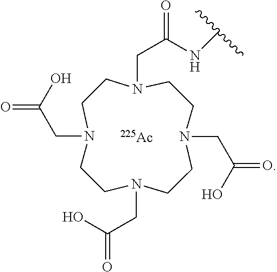

In some embodiments, the interaction of the metal chelator and a radionuclide of the compounds disclosed herein can be illustrated as

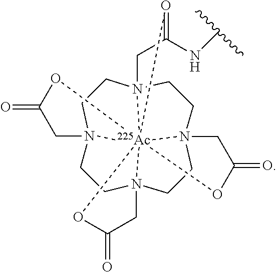

In some embodiments, the interaction of the metal chelator and a radionuclide of the compounds disclosed herein can be illustrated as

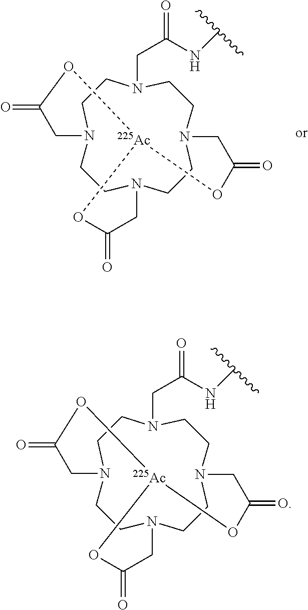

In some embodiments, the interaction of the metal chelator and a radionuclide of the compounds disclosed herein are illustrated as

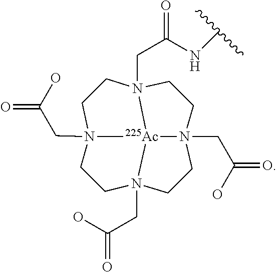

In some embodiments, the interaction of the metal chelator and a radionuclide of the compounds disclosed herein are illustrated as

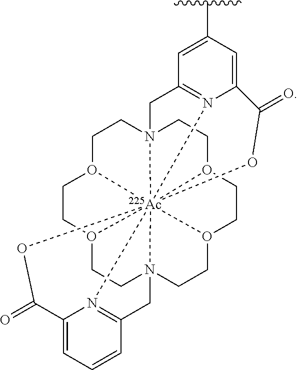

In some embodiments, the radionuclide exists in a positive oxidation state e.g., ²²⁵Ac³⁺, ¹⁷⁷Lu³⁺. In some embodiments, for example in certain aqueous conditions, the radionuclide exists in a salt form, e.g., as ²²⁵Ac³⁺, ¹⁷⁷Lu³⁺. In some embodiments, for example in certain acidic aqueous conditions, the radionuclide exists in a salt form, e.g., as ²²⁵Ac³⁺, ¹⁷⁷Lu³⁺. In some embodiments, the compound is in a salt form. In some embodiments, one or more of the carboxylic acid groups of the compound may exist as carboxylate anions. In some embodiments, one or more of the carboxylate anions of the compound may coordinate to the radionuclide. A person of ordinary skill would appreciate that the dissociation of an acid depends on the pH value of the environment and its pK value. Accordingly, in some embodiments, the compound described herein can exist in a completely ionized, partially ionized, or non-ionized form.

### Methods of Treatment and Compositions for Use in Such Methods

Disclosed herein are methods of treating a disease or disorder in a subject in need thereof, the method comprising administering to the subject a compound disclosed herein. In some embodiments, the disease or disorder is cancer.

Disclosed herein are methods of treating a CA-IX-mediated disease or disorder in a subject in need thereof, the method comprising administering to the subject a compound disclosed herein. In some embodiments, the CA-IX-mediated disease or disorder is cancer.

Disclosed herein are methods of treating cancer in a subject in need thereof, the method comprising administering to the subject a compound disclosed herein.

In some embodiments, the cancer is a solid tumor. In some embodiments, the cancer is a relapsed or refractory cancer. In some embodiments, the cancer is a metastatic cancer. In some embodiments, the cancer is hypoxic. In some embodiments, the cancer is clear cell renal cell carcinoma (ccRCC), In some embodiments, the cancer is clear cell renal cell carcinoma (ccRCC).

In some embodiments, provided herein are methods for killing a cell comprising contacting the cell with a compound or a pharmaceutically acceptable salt or stereoisomer thereof. In some embodiments, the cell expresses a CA-IX receptor. In some embodiments, the compound or a pharmaceutically acceptable salt or stereoisomer thereof releases a number of alpha particles by natural radioactive decay. In some embodiments, the compound or a pharmaceutically acceptable salt or stereoisomer thereof releases a number of beta particles, gamma rays, and/or Auger electrons by natural radioactive decay. The compounds described herein can kill a cell by radiation. In some embodiments, the compound kills the cell directly by radiation. In some embodiments, the compound kills the cell by inducing double-stranded DNA breaks. In some embodiments, the cell is a cancer cell. In some embodiments, the method comprises killing a cell with an alpha-particle emitting radionuclide. After contacting a cell, the compounds described herein can be internalized by the cell. The internalization can be mediated by cell receptors, cell membrane endocytosis, etc.

In addition to the methods of treatment described above, the compounds and compositions described herein can be used to diagnose and/or image, and/or as part of a treatment for diseases. Accordingly, also disclosed herein is a method of diagnosing or imaging a disease or disorder in a subject in need thereof, the method comprising administering to the subject a compound disclosed herein.

Compounds for diagnosis and/or imaging applications, e.g., single-photon emission computed tomography (SPECT) and positron emission tomography (PET), can comprise a radionuclide suitable for use as imaging isotopes. Accordingly, the compounds described herein can be administered as a companion diagnostic.

In some embodiments, disclosed herein are compounds of Table 1 or Table 2, or a salt (such as a pharmaceutically acceptable salt) or stereoisomer thereof, complexed with a contrast agent for use in magnetic resonance imaging (MRI). In certain embodiments, compounds of Table 1 or Table 2, or a salt (such as a pharmaceutically acceptable salt) or stereoisomer thereof, are complexed with a contrast agent selected from Gd-155, Mn-55, or Dy-161.

### Pharmaceutical Compositions and Routes of Administration

Also provided herein are pharmaceutical compositions comprising a compound of the present disclosure (e.g., a compound of structural formula (I), (la), (Ib), (II), (IIa), or (IIb) of Table 1 or Table 2, or a pharmaceutically acceptable salt thereof), and a pharmaceutically acceptable carrier, excipient or vehicle.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In some embodiments, the compounds described herein are administered to animals.

In another aspect, provided herein are pharmaceutical compositions comprising a compound described herein, or a pharmaceutically acceptable salt or stereoisomer thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

### EXAMPLES

The following Examples are presented by way of illustration, not limitation. One skilled in the art can modify the procedures set forth in the illustrative examples to arrive at the desired products.

The following abbreviations may be relevant for the application:

Abbreviations:

| | |
|---|---|
| Boc | tert-butyloxycarbonyl |
| ACN | acetonitrile |
| Boc₂O | Di-tert-butyl dicarbonate |
| BSA | bis(trimethylsilyl)acetamide |
| calcd. | calculated |
| DCC | N,N′-dicyclohexylcarbodiimide |
| DCE | 1,2-dichloroethane |
| DCM | dichloromethane |
| DIEA | N,N-diisopropylethylamine |
| DIPEA | N,N-diisopropylethylamine |
| DME | dimethyl ether |
| DMF | dimethyl formamide |
| DMSO | dimethyl sulfoxide |
| equiv | equivalent |
| EDCI | 1-ethyl-3-(3-dimethylaminopropyl)carbodiimide |
| ESI | electrospray ionization |
| EtOAc | ethyl acetate |
| EtOH | ethanol |
| FA | formic acid |
| Fmoc | fluorenylmethyloxycarbonyl |
| h | hour |
| HATU | O-(7-azabenzotriazol-1-yl)-N,N,N′,N′- |
| | tetramethyluronium hexafluorophosphate |
| HOAc | acetic acid |
| HPLC | high performance liquid chromatography |
| LAH | lithium aluminum hydride |
| LDA | lithium diisopropylamide |
| LED | light-emitting diode |
| LiOtBu | lithium tert-butoxide |
| MeCN | acetonitrile |
| MeOH | methanol |
| min | minute |
| MS | mass spectrometry |
| MTBE | methyl tert-butyl ether |
| NaAsc | sodium ascorbate |
| NaOAc | sodium acetate |
| NH₄OAc | ammonium acetate |
| NMP | N-methyl-2-pyrrolidone |
| NMR | nuclear magnetic resonance |
| Pb(OAc)₂ | lead (II) acetate |
| prep | preparative |
| Py | pyridine |
| rt | room temperature |
| RT | retention time |
| Su | succinimide |
| sat. | saturated |
| tBu | tert-butyl |
| t-BuOH | tert-butyl alcohol |
| t-BuOLi | lithium tert-butoxide |
| TEA | triethylamine |
| TFA | trifluoroacetic acid |
| TfOH | triflic acid |
| THF | tetrahydrofuran |
| TIPS | triisopropylsilane |
| TLC | thin layer chromatography |
| TMEDA | tetramethylethylenediamine |
| TMP | 2,2,6,6-tetramethylpiperidine |

### General Information

¹H NMR spectra were recorded on either a Bruker Avance III 400 (400 MHz), or Bruker Avance 300 (400 MHz) spectrometer. Chemical shifts are reported in ppm with solvent resonance as the internal standard (CDCl₃: 7.27 ppm, DMSO-d₆: 2.50 ppm, CD₃OD: 3.31 ppm). Data are reported as follows: chemical shift, integration, multiplicity (s=singlet, d=doublet, t=triplet, q=quartet, p=pentet, m=multiplet), and coupling constants (Hz).

NMR: Unless otherwise noted, ¹H NMR and 19F NMR spectra were acquired using 600 MHz spectrometers, 400 MHz spectrometers, 356 MHz spectrometers or 300 MHz spectrometers in a suitably selected deuterated solvent such as Dimethyl Sulfoxide-d₆, Chloroform-d, Methanol-d₄, Deuterium Oxide, Acetic Acid-d₄, Acetone-d₆, Acetonitrile-d₃, Benzene-d₆, Cyclohexane-d₁₂, N, N-Dimethyl-formamide-d₇, 1,4-Dioxane-d₈, Ethanol-d₆, Methylene Chloride-d₂, Pyridine-d₅, 1,1,2,2-Tetrachloroethane-d₂, Tetrahydrofuran-d₈, Toluene-d₈, Trifluoroacetic Acid-d, Trifluoroethanol-d₃, and the like. NMR data are reported in parts per million (8) and are referenced to the residual solvent signal of the deuterated solvent or TMS (Trimethylsilane). Coupling constants (J) are reported in hertz (Hz). The nature of the shifts as to multiplicity is reported as s (singlet), d (doublet), t (triplet), q (quartet), dd (doublet of doublets), ddd (doublet of doublet of doublets), dt (double of triplets), td (triplet of doublets), dq (doublet of quartets), tt (triplet of triplets), tdd (triplet of doublet of doublets), dtd (doublet of triplet of doublets), ddt (doublet of doublet of triplets), qd (quartet of doublets), qt (quartet of triplets), m (multiplet), br (broad). It will be understood that for compounds comprising an exchangeable proton, said proton may or may not be visible on an NMR spectrum depending on the choice of solvent used for running the NMR spectrum and the concentration of the compound in the solution.

Liquid chromatography was performed using forced flow (flash chromatography) on silica gel (SiO₂, 1000 mesh) or by column chromatography (silica gel, 1000 mesh). Thin layer chromatography (TLC) was performed on 20-25 μm silica gel glass backed plates. Preparative TLC was performed on a 40-45 μm silica gel glass backed plates. Visualization was performed using ultraviolet light (254 nm), iodide, or KMnO₄ in water.

### Example 1: Synthesis of Intermediates

### Intermediate A-1:5,5-Bis(4-hydroxyphenyl) hexanoic acid

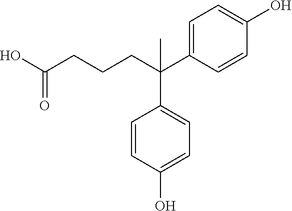 [SMILES: CC(CCCC(=O)O)(c1ccc(O)cc1)c1ccc(O)cc1]

This reaction was run in triplicate. To a solution of 5-oxohexanoic acid (100 g, 0.76 mol, 92 mL) in H₂O (25 mL, 1.5 mol) was added phenol (145 g, 1.54 mol, 135 mL). The reaction mixture was then cooled to 0° C. and H₂SO₄ (74 mL, 1.4 mol) was added dropwise over 15 minutes. The reaction mixture was then warmed to 50° C. and stirred at this temperature for 96 hours. The reaction mixture was poured into water (2 L) and stirred for 10 minutes. The aqueous phase was extracted with ethyl acetate (2 L×3) and the combined organic layers were washed with water (1 L×6). The combined aqueous layers were extracted with ethyl acetate (1 L×2) and the combined organic layers were dried over Na₂SO₄, filtered and concentrated under reduced pressure. The residue was purified by flash column chromatography (SiO₂, Petroleum ether/Ethyl acetate=10/1 to 1/1) and then further purified by prep-HPLC (column: Phenomenex Titan C18 Bulk 250×150 mm, 10 μm; mobile phase: [H₂O (10 mM NH₄HCO₃)-ACN]; gradient: 1%-25% B over 20.0 min) to afford the title compound (128 g, 18% yield) as a white solid. ¹H NMR (400 MHz, DMSO-d₆) δ 11.95 (br s, 1H), 9.13 (br s, 2H), 6.94 (d, J=8.6 Hz, 4H), 6.64 (d, J=8.6 Hz, 4H), 2.16 (br t, J=7.4 Hz, 2H), 2.03-1.85 (m, 2H), 1.48 (s, 3H), 1.35-1.16 (m, 2H).

### Intermediate A-2: Tert-butyl 4-amino-4-((2-azidoethyl) carbamoyl) piperidine-1-carboxylate

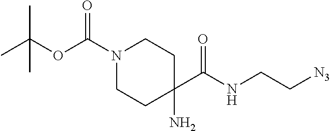 [SMILES: CC(C)(C)OC(=O)N1CCC(N)(C(=O)NCCN=[N+]=[N-])CC1]

Step A: Tert-butyl 4-((((9H-fluoren-9-yl) methoxy) carbonyl)amino)-4-((2-azidoethyl) carbamoyl) piperidine-1-carboxylate. Eight reactions were carried out in parallel. To a solution of 4-((((9H-fluoren-9-yl) methoxy) carbonyl)amino)-1-(tert-butoxycarbonyl) piperidine-4-carboxylic acid (50 g, 107 mmol) in DMF (500 mL) was added DIPEA (56 mL, 322 mmol) and HATU (61 g, 161 mmol) followed by 2-azidoethan-1-amine hydrochloride (14 g, 113 mmol). The reaction mixture was stirred at 20° C. for 1 hour then poured into water (1000 mL). The aqueous phase was extracted with ethyl acetate (1000 mL×2). The combined organic layers were washed with a saturated aqueous solution of NaCl (800 mL×2), dried over Na₂SO₄, filtered and concentrated under reduced pressure. The residue was triturated with MTBE (1 L) at 20° C. for 20 minutes to afford the title compound (480 g, 96% yield) as a pink solid. MS (ESI): mass calcd. for C₂₈H₃₄N₆O₅, 534.3; m/z found, 435.3 [M-tBu+H]⁺. ¹H NMR (400 MHz, CDCl₃-d) δ 7.78 (d, J=7.6 Hz, 2H), 7.58 (d, J=7.4 Hz, 2H), 7.46-7.39 (m, 2H), 7.37-7.30 (m, 2H), 6.85 (br s, 1H), 4.97 (br s, 1H), 4.54 (br d, J=1.0 Hz, 2H), 4.20 (t, J=6.0 Hz, 1H), 3.89-3.69 (m, 2H), 3.38 (br s, 4H), 3.07-2.93 (m, 2H), 2.11-1.78 (m, 4H), 1.46 (s, 9H).

Step B: Tert-butyl 4-amino-4-((2-azidoethyl) carbamoyl) piperidine-1-carboxylate. Three reactions were carried out in parallel. To a solution of tert-butyl 4-((((9H-fluoren-9-yl) methoxy) carbonyl)amino)-4-((2-azidoethyl) carbamoyl) piperidine-1-carboxylate (150 g, 281 mmol) in DCM (1500 mL) was added N-ethylethanamine (145 mL, 1.40 mol) and the reaction was stirred at 25° C. for 2 hours. The reaction mixture was concentrated under reduced pressure and the residue was diluted with H₂O (30 mL). The pH was adjusted to pH 4 using HCl (1M). The aqueous phase was then extracted with EtOAc (100 mL×3). The aqueous phase was adjusted to pH 8 by using a saturated aqueous solution of NaHCO₃. The aqueous phase was extracted with EtOAc (200 mL×3) and the combined organic layers were washed with a saturated aqueous solution of NaCl (200 mL), dried over Na₂SO₄, filtered and concentrated under reduced pressure to afford the title compound (190 g, 73% yield) as a yellow oil which was used in the next step without further purification.

### Intermediate A-3

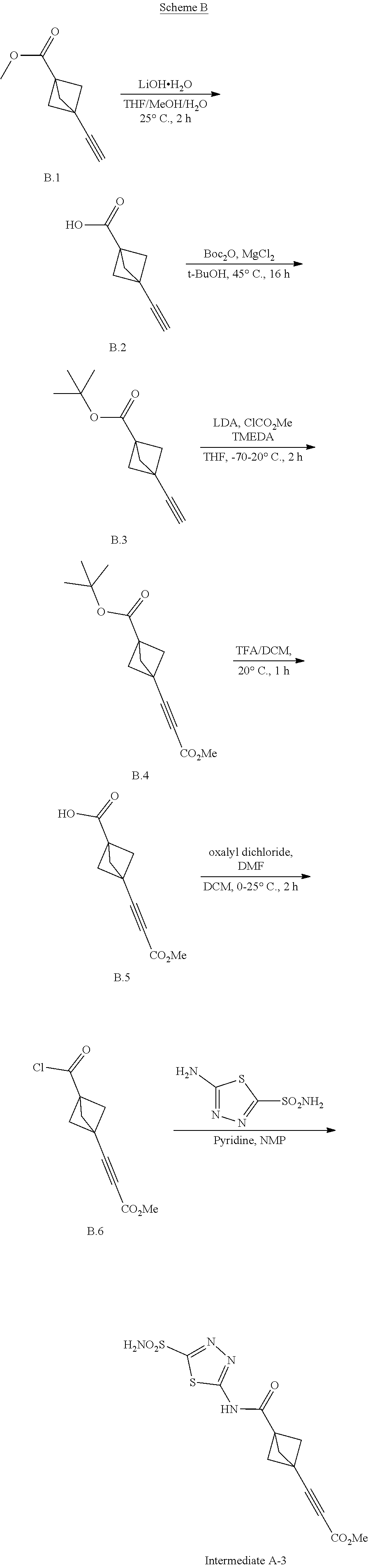

Compound B.2. To a solution of Compound B.1 (30 g, 190 mmol) in THF (100 mL), MeOH (100 mL) and H₂O (100 mL) was added LiOH·H₂O (6 g, 142 mmol). The mixture was stirred at 25° C. for 2 hours and then concentrated under reduced pressure to give a residue. The residue was diluted with H₂O (20 mL) and the pH was adjusted to pH 5 using 1N HCl. The aqueous phase was extracted with EtOAc (200 mL×3) and the combined organic layers were washed with a saturated aqueous solution of NaCl (200 mL), dried over Na₂SO₄, filtered and concentrated under reduced pressure to afford Compound B.2 (25 g, 96% yield) as a white solid. ¹H NMR (400 MHz, CDCl₃-d) δ 9.80-9.40 (m, 1H), 2.36 (s, 6H), 2.13 (s, 1H).

Compound B.3. To a solution of Compound B.2 (23 g, 169 mmol) in t-BuOH (220 mL) was added MgCl₂ (1.6 g, 17 mmol, 693 μL) and Boc₂O (74 g, 338 mmol). The reaction mixture was stirred at 45° C. for 16 hours then a saturated aqueous solution of NaHCO₃ (200 mL) was added at 25° C. The aqueous layers were extracted with DCM (60 mL), dried over Na₂SO₄, filtered and concentrated under reduced pressure to give a residue. The water layers were adjusted to pH 3 with HCl 2M (100 mL) and extracted with EtOAc (20 mL). The organic layer was dried over Na₂SO₄, filtered and concentrated under reduced pressure to give a residue. Both residues were combined and purified by flash column chromatography (SiO₂, Petroleum ether/Ethyl acetate=100/1 to 80/1) to afford Compound B.3 (54 g) as a white solid. ¹H NMR (400 MHz, CDCl₃-d) δ 2.28 (s, 6H), 2.11 (s, 1H), 1.44 (s, 9H).

Compound B.4. To a solution of Compound B.3 (15.4 g, 76.1 mmol) in THF (1540 mL) in a 3 L three-port flask was added TMEDA (884 mg, 7.61 mmol, 1.15 mL) at 20° C. in one portion. The reaction mixture was purged with N₂ three times and cooled to −70° C. followed by the drop wise addition of LDA (2 M, 69 mL) over 10 minutes. After 30 minutes, methyl carbonochloridate (7.9 g, 84 mmol, 6.5 mL) was added dropwise to the reaction mixture at −70° C. over 10 minutes then the reaction mixture was stirred at 20° C. for 1 hour. To the reaction mixture was added a saturated aqueous solution of NH₄Cl (400 mL) at 20° C. and stirred for 30 minutes. The aqueous phase was extracted with EtOAc (700 mL×3) and the combined organic layers were dried over Na₂SO₄, filtered and concentrated under reduced pressure to give a residue. The residue was purified by column chromatography (SiO₂, Petroleum ether/Ethyl acetate=100/0 to 95/5) to afford Compound B.4 (37 g) as a white solid. ¹H NMR (400 MHz, CDCl₃-d) δ 3.78 (s, 3H), 2.34 (s, 6H), 1.44 (s, 9H).

Compound B.5. To a solution of Compound B.4 (27 g, 108 mmol) in DCM (140 mL) was added TFA (140 mL). The reaction mixture was stirred at 20° C. for 2 hours then the mixture concentrated under reduced pressure to afford Compound B.5 (18 g, 86% yield) as a white solid. ¹H NMR (400 MHz, CDCl₃-d) δ 3.78 (s, 3H), 2.44 (s, 6H).

Compound B.6. To a solution of Compound B.5 (18 g, 93 mmol) in DCM (300 mL) was added DMF (713 μL, 9.3 mmol). The reaction mixture was cooled to 0° C. then oxalyl dichloride (9.8 mL, 111 mmol) was added. The mixture was stirred at 20° C. for 1 hour and then concentrated under reduced pressure to afford Compound B.6 (19 g, 96% yield) as a white solid.

Intermediate A-3. To a solution of Compound B.6 (16 g, 89 mmol) in NMP (300 mL) was added pyridine (14.4 mL, 178 mmol). The reaction mixture was cooled to 0° C. then a solution of 5-amino-1,3,4-thiadiazole-2-sulfonamide (19 g, 89 mmol) in NMP (300 mL) was added under an atmosphere of N₂. The reaction mixture was stirred at 20° C. for 1 hour then concentrated under reduced pressure to give a residue. The residue was triturated with H₂O at 25° C. for 15 minutes, then the mixture was filtered and concentrated under reduced pressure to afford Intermediate A-3 (31 g, 90% yield) as a white solid. ¹H NMR (400 MHz, DMSO-d₆) δ 13.24 (br s, 1H), 8.34 (s, 2H), 3.71 (s, 3H), 2.52 (s, 6H).

### Intermediate A-4

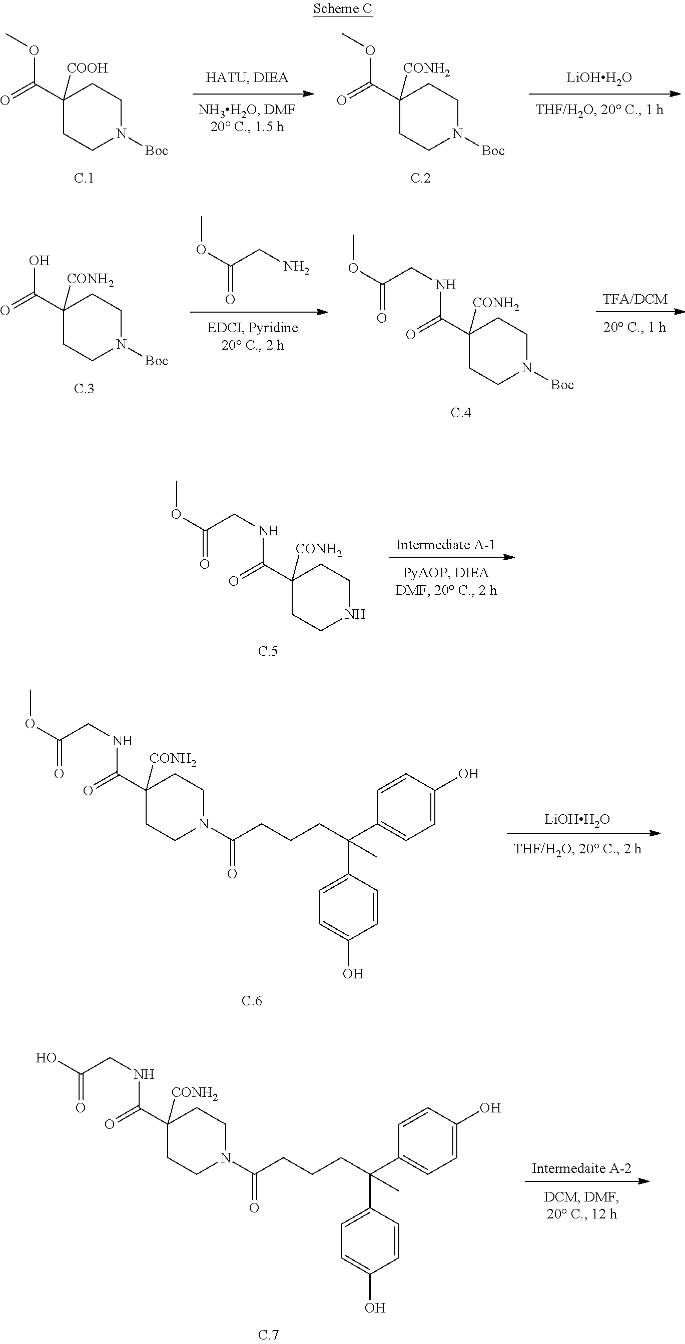

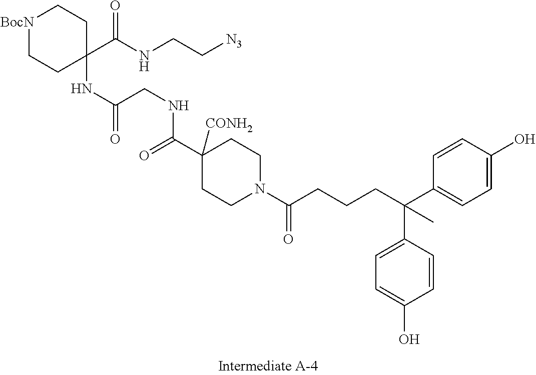 [SMILES: CC(C)(C)OC(=O)N1CCC(NC(=O)CNC(=O)C2(C(N)=O)CCN(C(=O)CCCC(C)(c3ccc(O)cc3)c3ccc(O)cc3)CC2)(C(=O)NCCN=[N+]=[N-])CC1]

Compound C.2. To a solution of Compound C.1 (60 g, 209 mmol) in DMF (500 mL) was added HATU (83.4 g, 219 mmol) and DIEA (109 mL, 627 mmol). The mixture was stirred at 20° C. for 30 minutes then NH₃·H₂O (31.4 g, 251 mmol, 34.5 mL) was added and the reaction mixture was stirred for 1 hour. The reaction mixture was extracted with ethyl acetate (500 mL×4) and the combined organic layers were washed with a saturated aqueous solution of NaCl (200 mL×3), dried over Na₂SO₄, filtered and concentrated under reduced pressure to give a residue. The residue was purified by flash column chromatography (SiO₂, DCM/MeOH=100/0 to 95/5) to afford Compound C.2 (60 g, 92% yield) as a white solid. MS (ESI): mass calcd. for C₁₃H₂₂N₂O₅, 286.2; m/z found, 287.2 [M+H]⁺. ¹H NMR (400 MHz, DMSO-d₆) δ 7.45-7.14 (m, 2H), 3.65 (s, 3H), 3.52-3.39 (m, 2H), 3.10 (br s, 2H), 1.98-1.80 (m, 4H), 1.48-1.29 (m, 9H).

Compound C.3. To a solution of Compound C.2 (50 g, 175 mmol) in THF (300 mL) and H₂O (100 mL) was added LiOH·H₂O (15 g, 349 mmol) at 20° C. The reaction mixture was stirred for 1 hour then was concentrated under reduced pressure to remove THF. To the aqueous phase was added HCl (1 N) and the pH was adjusted to pH 2-3. The mixture was filtered and concentrated under reduced pressure to afford Compound C.3 (57 g) as a white solid. MS (ESI): mass calcd. for C₁₂H₂₀N₂O₅, 272.1; m/z found, 273.1 [M+H]⁺. ¹H NMR (400 MHz, DMSO-d₆) δ 12.91-12.68 (m, 1H), 7.19 (s, 2H), 3.40 (ddd, J=4.0, 6.2, 13.4 Hz, 2H), 3.15 (br d, J=8.8 Hz, 2H), 1.96-1.78 (m, 4H), 1.38 (s, 9H).

Compound C.4. To a solution of Compound C.3 (55 g, 101 mmol) and methyl glycinate (28 g, 111 mmol) in pyridine (400 mL) was added EDCI (46.5 g, 121 mmol) at 20° C. The reaction mixture was stirred at 20° C. for 2 hours and concentrated under reduced pressure to give a residue. The residue was purified by flash column chromatography (SiO₂, DCM/MeOH=100/0 to 98/2) to afford Compound C.4 (67 g) as a white solid. ¹H NMR (400 MHz, DMSO-d₆) δ 8.13 (br t, J=5.6 Hz, 1H), 7.30-6.99 (m, 2H), 3.82 (br d, J=5.5 Hz, 2H), 3.62 (s, 3H), 3.50-3.37 (m, 2H), 3.16 (br s, 2H), 2.08-1.91 (m, 2H), 1.88-1.73 (m, 2H), 1.39 (s, 9H).

Compound C.5. To a solution of Compound C.4 (60 g, 175 mmol) in DCM (300 mL) was added TFA (100 mL) at 20° C. and the reaction mixture was stirred for 1 hour. The reaction mixture was concentrated under reduced pressure and the residue was triturated with THF at 20° C. for 10 minutes to afford Compound C.5 (53 g, 82% yield, TFA) as a white solid. ¹H NMR (400 MHz, DMSO-d₆) δ 8.51-8.37 (m, 2H), 7.52-7.11 (m, 2H), 3.85 (br d, J=5.8 Hz, 2H), 3.64 (s, 3H), 3.11 (br s, 2H), 3.00 (br s, 2H), 2.24 (br d, J=13.6 Hz, 2H), 2.11-1.99 (m, 2H).

Compound C.6. To a solution of Compound C.5 (40 g, 112 mmol) and Intermediate A-1 (34 g, 112 mmol) in DMF (300 mL) was added DIEA (58.5 mL, 336 mmol) and (7-azabenzotriazol-1-yloxy)trispyrrolidinophosphonium hexafluorophosphate (PyAOP) (61.3 g, 118 mmol) at 20° C. The reaction mixture was stirred for 2 hours then H₂O (600 mL) was added and the aqueous phase was extracted with ethyl acetate (300 mL×3). The combined organic layers were washed with a saturated aqueous solution of NaCl (150 mL×3), dried over Na₂SO₄, filtered and concentrated under reduced pressure to give a residue. The residue was purified by flash column chromatography (SiO₂, DCM/MeOH=100/0 to 94/6) to give Compound C.6 (60 g, 89% yield) as a white solid. MS (ESI): mass calcd. for C₂₈H₃₅N₃O₇, 525.3; m/z found, 526.2 [M+H]⁺. ¹H NMR (400 MHz, DMSO-d₆) δ 9.15-9.07 (m, 2H), 8.15 (t, J=5.8 Hz, 1H), 7.26-7.04 (m, 2H), 6.97-6.89 (m, 4H), 6.63 (d, J=8.6 Hz, 4H), 3.83 (d, J=5.8 Hz, 2H), 3.62 (s, 3H), 3.47-3.39 (m, 1H), 3.29-3.22 (m, 1H), 3.17 (d, J=5.3 Hz, 2H), 2.29-2.21 (m, 2H), 2.07-1.90 (m, 4H), 1.88-1.72 (m, 2H), 1.48 (s, 3H), 1.26 (d, J=1.1 Hz, 2H).

Compound C.7. To a solution of Compound C.6 (40 g, 76 mmol) in THF (120 mL) and H₂O (40 mL) was added LiOH·H₂O (6.4 g, 152 mmol) at 20° C. The reaction mixture was stirred for 2 hours and then concentrated under reduced pressure to remove THF. The pH was adjusted to pH 2-3 using HCl (1N aqueous solution). The mixture was filtered and concentrated under reduced pressure to afford Compound C.7 (36 g, 93% yield) as a white solid. MS (ESI): mass calcd. for C₂₇H₃₃N₃O₇, 511.2; m/z found, 512.2 [M+H]⁺. ¹H NMR (400 MHz, DMSO-d₆) § 12.07-13.19 (m, 1H) 9.00-9.36 (m, 2H) 8.09 (br t, J=5.6 Hz, 1 H) 7.12-7.25 (m, 2H) 6.93 (d, J=8.6 Hz, 4H) 6.63 (d, J=8.6 Hz, 4H) 3.74 (br d, J=5.75 Hz, 2H) 3.57-3.66 (m, 1H) 3.45 (br s, 1H) 3.19-3.29 (m, 2H) 2.24 (br t, J=7.2 Hz, 2H) 1.89-2.06 (m, 4H) 1.74-1.87 (m, 2H) 1.47 (s, 3H) 1.17-1.30 (m, 2H).

Intermediate A-4. To a solution of Compound C.7 (46 g, 90 mmol, 1 eq) in DMF (460 mL) was added Intermediate A-2 (31 g, 99 mmol) and DCC (28 g, 135 mmol) at 20° C. The reaction mixture was stirred for 12 hours and then diluted with water (1000 mL). The aqueous phase was extracted with ethyl acetate (1000 mL×3) and the combined organic layers were washed with a saturated aqueous solution of NaCl (1000 mL), dried over Na₂SO₄, filtered and evaporated under reduced pressure to give a yellow residue. The residue was triturated with MeOH at 25° C. for 30 minutes three times to afford Intermediate A-4 (46 g, 63% yield) as a white solid. MS (ESI): mass calcd. for C₄₀H₅₅N₉O₉, 805.4; m/z found, 806.4 [M+H]⁺. ¹H NMR (400 MHz, DMSO-d₆) δ 9.12 (s, 2H), 8.27 (br t, J=5.4 Hz, 1H), 8.10 (s, 1H), 7.67 (t, J=5.6 Hz, 1H), 7.34 (br d, J=19.8 Hz, 2H), 6.94 (d, J=8.6 Hz, 4H), 6.63 (d, J=8.6 Hz, 4H), 5.57 (br d, J=8.0 Hz, 2H), 3.89-3.66 (m, 5H), 3.55 (br d, J=13.8 Hz, 1H), 3.33-3.30 (m, 2H), 3.24-3.20 (m, 2H), 3.04-2.91 (m, 2H), 2.25 (br t, J=7.2 Hz, 2H), 2.14-2.02 (m, 2H), 2.01-1.90 (m, 4H), 1.72 (br dd, J=3.9, 8.4 Hz, 4H), 1.48 (s, 3H), 1.40 (s, 9H), 1.32-1.26 (m, 2H).

### Intermediate A-5

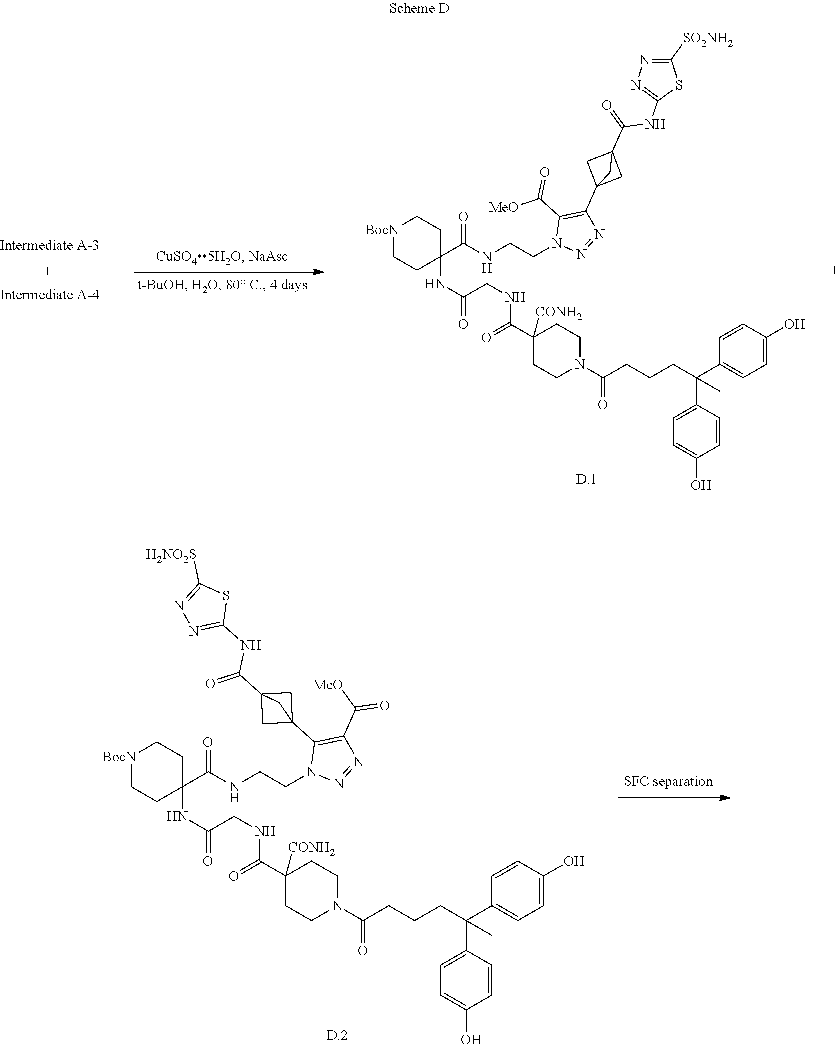 [SMILES: COC(=O)c1c(C23CC(C(=O)Nc4nnc(S(N)(=O)=O)s4)(C2)C3)nnn1CCNC(=O)C1(NC(=O)CNC(=O)C2(C(N)=O)CCN(C(=O)CCCC(C)(c3ccc(O)cc3)c3ccc(O)cc3)CC2)CCN(C(=O)OC(C)(C)C)CC1 ; COC(=O)c1nnn(CCNC(=O)C2(NC(=O)CNC(=O)C3(C(N)=O)CCN(C(=O)CCCC(C)(c4ccc(O)cc4)c4ccc(O)cc4)CC3)CCN(C(=O)OC(C)(C)C)CC2)c1C12CC(C(=O)Nc3nnc(S(N)(=O)=O)s3)(C1)C2]

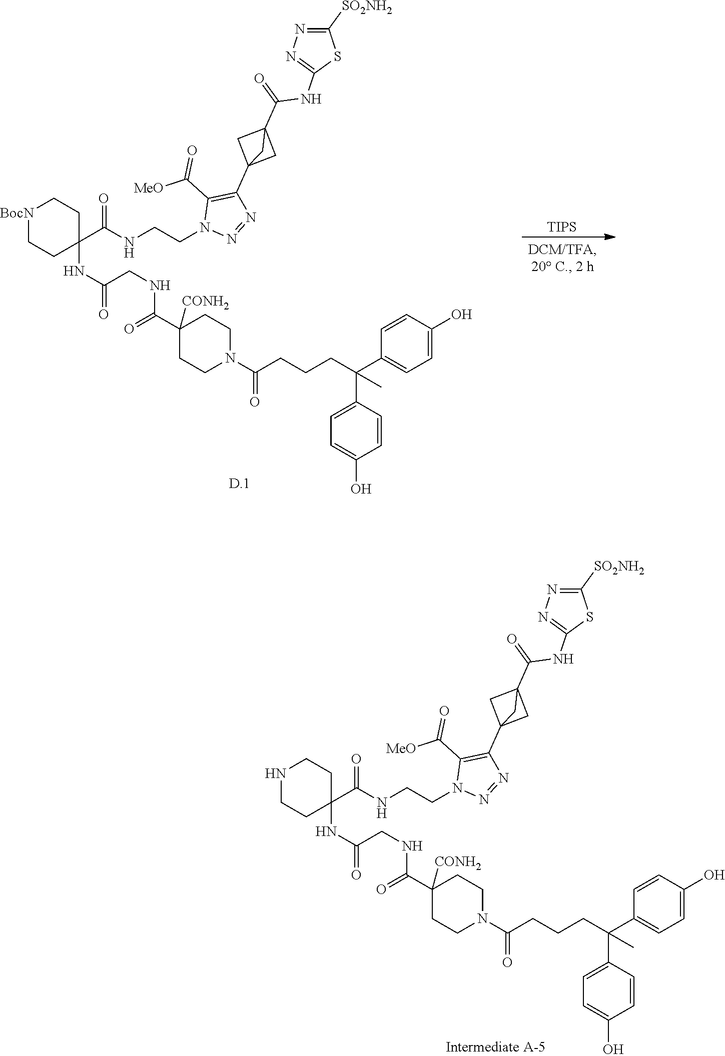 [SMILES: COC(=O)c1c(C23CC(C(=O)Nc4nnc(S(N)(=O)=O)s4)(C2)C3)nnn1CCNC(=O)C1(NC(=O)CNC(=O)C2(C(N)=O)CCN(C(=O)CCCC(C)(c3ccc(O)cc3)c3ccc(O)cc3)CC2)CCN(C(=O)OC(C)(C)C)CC1 ; COC(=O)c1c(C23CC(C(=O)Nc4nnc(S(N)(=O)=O)s4)(C2)C3)nnn1CCNC(=O)C1(NC(=O)CNC(=O)C2(C(N)=O)CCN(C(=O)CCCC(C)(c3ccc(O)cc3)c3ccc(O)cc3)CC2)CCNCC1 ; "TIPS"]

Compound D.1 and Compound D.2. To a solution of Intermediate A-4 (25 g, 14 mmol) and Intermediate A-3 (17 g, 21 mmol) in t-BuOH (50 mL) and H₂O (50 mL) under N2 was added CuSO₄·5H₂O (350 mg, 1.40 mmol) and sodium ascorbate (1.1 g, 5.6 mmol). The reaction mixture was stirred at 80° C. for 4 days and concentrated under reduced pressure. The reaction mixture was filtered to give a residue which was purified by prep-HPLC (column: Welch Xtimate C18 250×70 mm, 10 μm; mobile phase: [H₂O (10 mM NH₄HCO₃)-ACN]; gradient: 15%-45% B over 20.0 minutes) to afford a mixture of Compound D.1 and Compound D.2 (47 g, 85% yield) as a white solid. The mixture (21.7 g) was separated by prep-SFC (column: REGIS (s,s) WHELK-O1 (250 mm×50 mm, 10 μm); mobile phase: [CO₂-EtOH(0.1% NH₃H₂O)]; B %: 60%, isocratic elution mode) to afford the desired Compound D.1 (12 g, 55% yield) as a white solid. ¹H NMR (400 MHz, DMSO-d₆) δ 13.22 (br s, 1H), 9.11 (s, 2H), 8.34 (br s, 2H), 8.22-8.12 (m, 1H), 8.04 (s, 1H), 7.62 (br t, J=5.8 Hz, 1H), 7.41-7.23 (m, 2H), 6.93 (d, J=8.6 Hz, 4H), 6.62 (d, J=8.6 Hz, 4H), 4.65 (br t, J=5.4 Hz, 2H), 4.34 (d, J=4.2 Hz, 3H), 3.95 (s, 3H), 3.86-3.71 (m, 5H), 3.70-3.56 (m, 2H), 3.49 (br s, 3H), 3.05-2.83 (m, 3H), 2.53 (s, 5H), 2.24 (br t, J=6.8 Hz, 2H), 1.93 (br dd, J=6.2, 9.2 Hz, 4H), 1.88-1.72 (m, 4H), 1.69-1.57 (m, 2H), 1.47 (s, 3H), 1.38 (s, 9H).

Intermediate A.5. To a solution of Compound D.1 (12 g, 7.9 mmol) in DCM (60 mL) was added triisopropylsilane (32.2 mL, 157 mmol) and TFA (60 mL). The reaction mixture was stirred at 20° C. for 2 hours then concentrated under reduced pressure. The residue was purified by prep-HPLC (column: Phenomenex luna C18 250×150 mm, 15 μm; mobile phase: [H₂O (0.1% TFA)-ACN]; gradient: 5%-35% B over 20.0 min) to afford Intermediate A.5 (7.6 g, 90% yield, 99% purity) as a white solid. ¹H NMR (400 MHz, DMSO-d₆) δ 13.24 (s, 1H), 9.14 (br s, 2H), 8.60-8.46 (m, 1H), 8.43-8.26 (m, 4H), 8.19 (br t, J=5.2 Hz, 1H), 7.80 (br t, J=5.8 Hz, 1H), 7.32 (br s, 2H), 6.93 (d, J=8.6 Hz, 4H), 6.62 (d, J=8.6 Hz, 4H), 4.66 (br t, J=5.2 Hz, 2H), 3.95 (s, 3H), 3.85-3.67 (m, 3H), 3.48 (br s, 2H), 3.25-3.08 (m, 3H), 3.06-2.85 (m, 3H), 2.54 (s, 6H), 2.25 (br t, J=7.0 Hz, 2H), 2.00 (br s, 6H), 1.96-1.89 (m, 2H), 1.88-1.68 (m, 2H), 1.47 (s, 3H), 1.22 (br s, 2H).

### Intermediate A-6

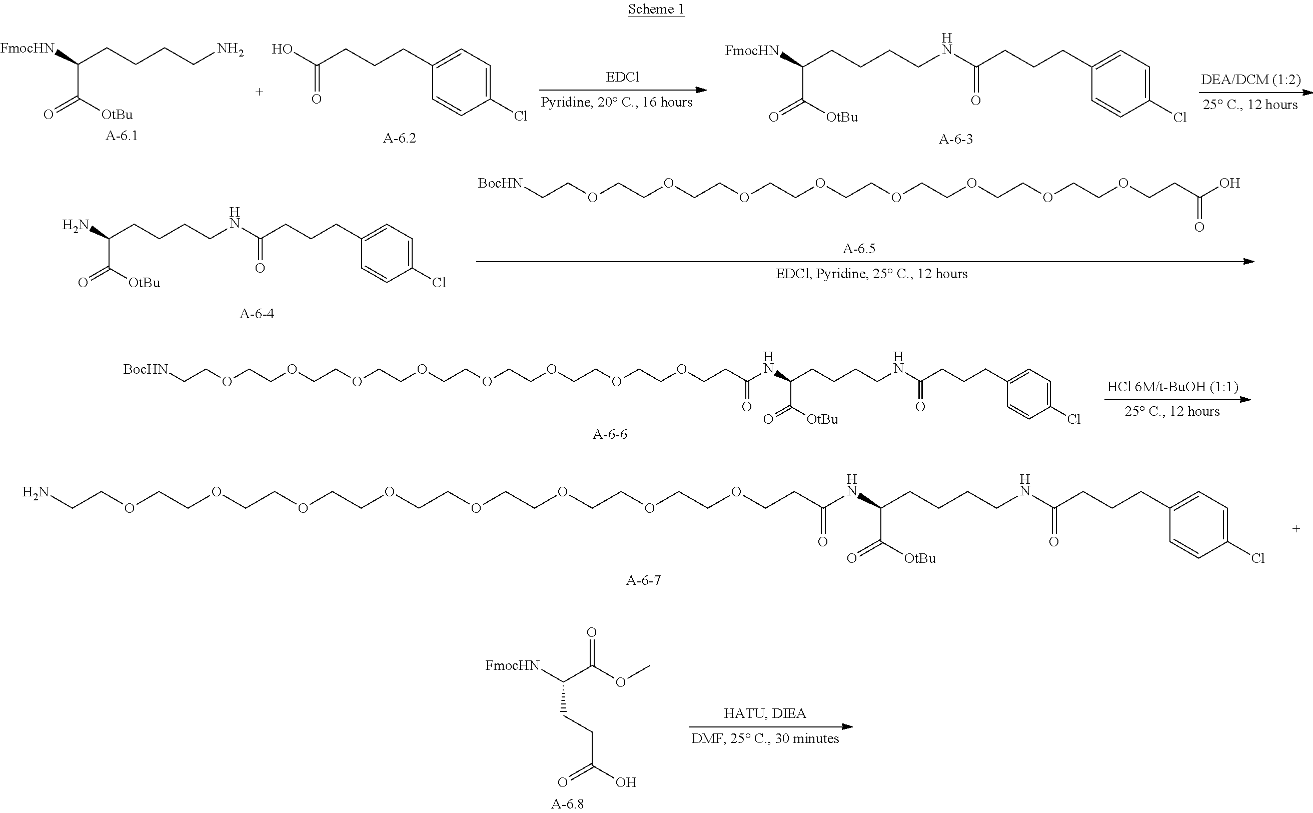

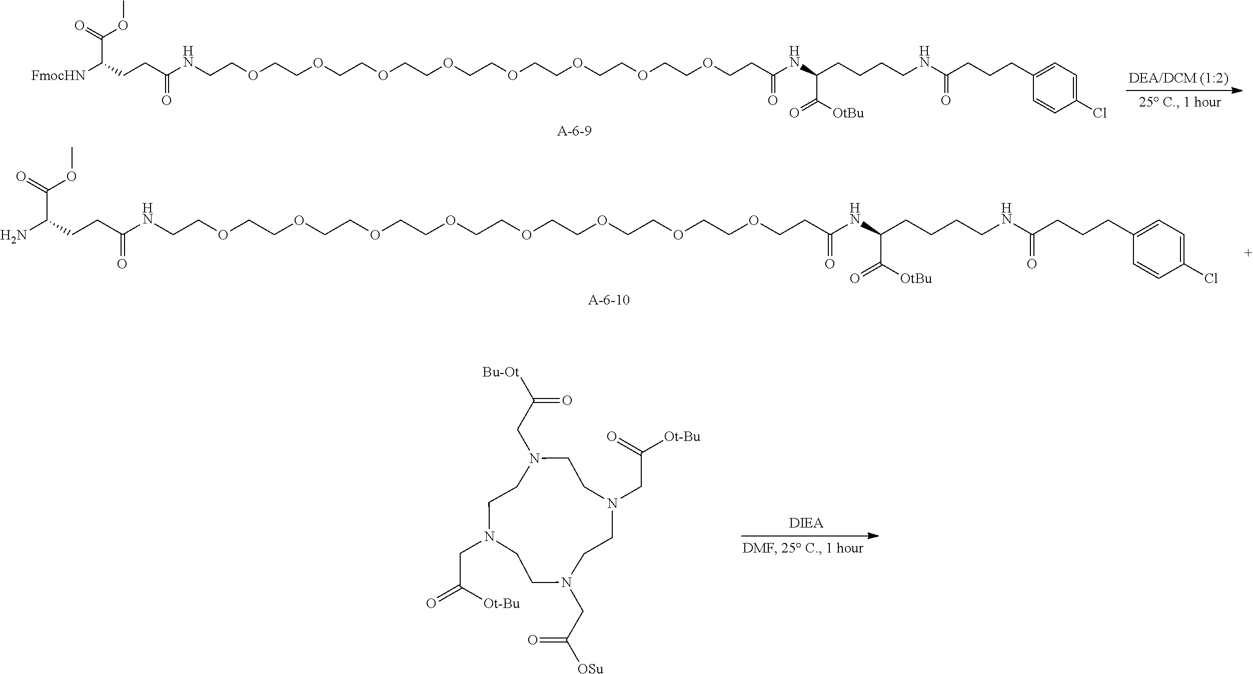

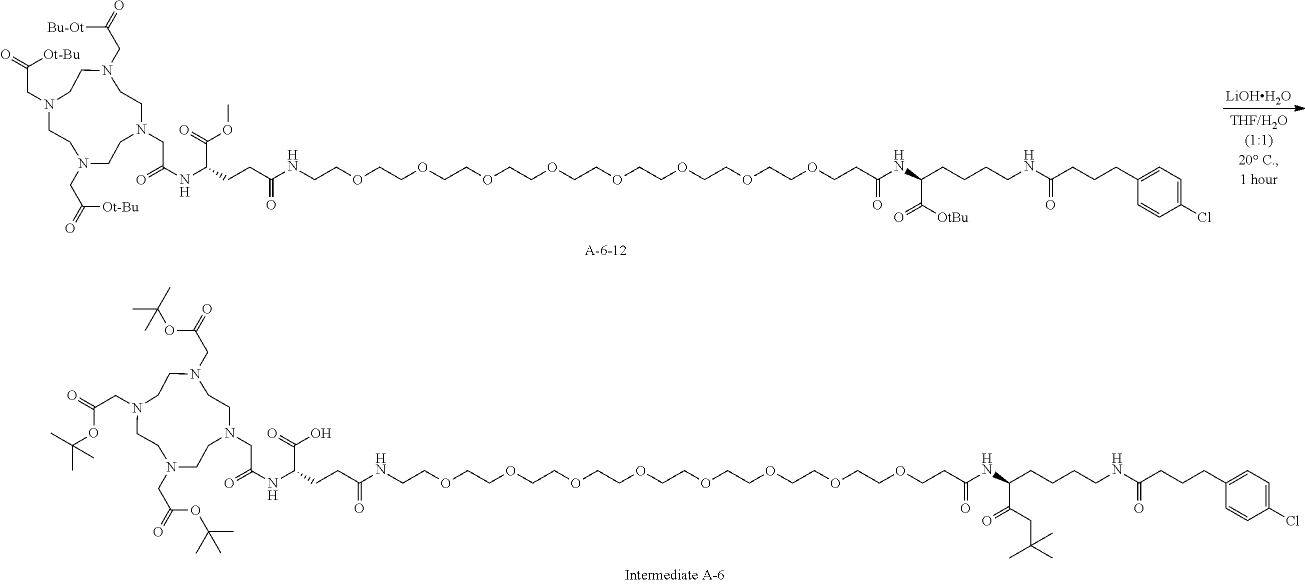

Intermediate A-6.3: To a solution of Intermediate A-6.1 (20 g, 43 mmol, HCl salt) in pyridine (200 mL) was added EDCI (25 g, 130 mmol) and Intermediate A-6.2 (8.6 g, 43 mmol). The reaction mixture was stirred at 20° C. for 16 hours then concentrated under reduced pressure. The residue was purified by flash column chromatography (SiO₂, Petroleum ether/Ethyl acetate=1/0 to 2/1) to afford Intermediate A-6.3 (25 g, 96% yield) as a yellow oil. MS (ESI): mass calcd. for C₃₅H₄₁ClN₂O₅, 604.3; m/z found, 605.4 [M+H]⁺. ¹H NMR (400 MHz, CDCl₃-d) δ 7.78 (d, J=7.6 Hz, 2H), 7.60 (br d, J=7.4 Hz, 2H), 7.44-7.38 (m, 2H), 7.36-7.29 (m, 2H), 7.22 (br d, J=8.2 Hz, 2H), 7.07 (br d, J=8.2 Hz, 2H), 5.51 (br s, 1H), 5.42 (br d, J=8.0 Hz, 1H), 4.44-4.30 (m, 2H), 4.30-4.18 (m, 2H), 3.24 (qd, J=6.2, 12.4 Hz, 2H), 2.58 (br t, J=7.4 Hz, 2H), 2.16-2.08 (m, 2H), 1.92 (quin, J=7.4 Hz, 2H), 1.87-1.78 (m, 1H), 1.75-1.64 (m, 1H), 1.54 (br d, J=6.4 Hz, 2H), 1.48 (s, 9H), 1.43-1.33 (m, 2H).

Intermediate A-6.4: A solution of Intermediate A-6.3 (15 g, 25 mmol) in DEA (50 mL) and DCM (100 mL) was stirred at 25° C. for 12 hours. This reaction mixture was combined with another reaction mixture of the same reaction for work up and was concentrated under reduced pressure to give a residue. The pH of the residue was adjusted to pH=5 using HCl (1 M) then the aqueous solution was extracted with MTBE (150 mL×2). The aqueous phase was adjusted to pH=8 using a saturated aqueous solution of NaHCO₃then the aqueous solution was extracted with DCM (100 mL×3). The combined organic layers were washed with a saturated aqueous solution of NaCl (100 mL×2), dried over with Na₂SO₄, filtered and concentrated under reduced pressure to afford Intermediate A-6.4 (16 g, crude) as a yellow oil. MS (ESI): mass calcd. for C₂₀H₃₁ClN₂O₃, 382.2; m/z found, 383.3 [M+H]⁺.

Intermediate A-6.6: To a solution of Intermediate A-6.4 (10 g, 26 mmol) in pyridine (100 mL) was added EDCI (15 g, 78 mmol) and Intermediate A-6.5 (14 g, 26 mmol) at 25° C. The reaction mixture was stirred for 12 hours then concentrated under reduced pressure to give a residue. The residue was diluted with H₂O (150 mL) and extracted with DCM (100 mL×3). The combined organic layers were washed with a saturated aqueous solution of NaCl (50 mL×3), dried over Na₂SO₄, filtered and concentrated under reduced pressure to give a residue which was purified by flash column chromatography (SiO₂, Dichloromethane: Methanol=1/0 to 10/1) to afford Intermediate A-6.6 (20.5 g, 87% yield) as a yellow oil. MS (ESI): mass calcd. for C₄₄H₇₆ClN₃O₁₄, 905.5; m/z found, 906.6 [M+H]⁺. ¹H NMR (400 MHz, CDCl₃-d) δ 7.26-7.21 (m, 2H), 7.11 (d, J=8.4 Hz, 2H), 6.82 (br d, J=8.0 Hz, 1H), 5.82 (br s, 1H), 5.11-5.00 (m, 1H), 3.80 (t, J=7.0 Hz, 1H), 3.76-3.71 (m, 2H), 3.66-3.63 (m, 26H), 3.54 (t, J=5.2 Hz, 2H), 3.38-3.27 (m, 3H), 3.26-3.18 (m, 2H), 2.62 (t, J=7.6 Hz, 2H), 2.48 (t, J=5.8 Hz, 2H), 2.20-2.11 (m, 2H), 1.98-1.90 (m, 2H), 1.90-1.86 (m, 1H), 1.84-1.76 (m, 1H), 1.70-1.60 (m, 1H), 1.57-1.49 (m, 2H), 1.45 (d, J=3.6 Hz, 18H), 1.40-1.32 (m, 2H).

Intermediate A-6.7: To a solution of Intermediate A-6.6 (5 g, 5.5 mmol) in 1-BuOH (25 mL) was added HCl 6M (25 mL). The reaction mixture was stirred at 25° C. for 1 hour then the pH was adjusted pH to 7 by addition of NaHCO₃ at 25° C. The reaction mixture was concentrated under reduced pressure and the residue was diluted with H₂O (50 mL) and extracted with DCM (100 mL×3). The combined organic layers were washed with a saturated aqueous solution of NaCl (50 mL×3), dried over Na₂SO₄, filtered and concentrated under reduced pressure to afford give Intermediate A-6.7 (4.6 g) as a yellow oil. MS (ESI): mass calcd. for C₃₉H₆₈ClN₃O₁₂, 805.5; m/z found, 806.6 [M+H]⁺. ¹H NMR (400 MHz, DMSO-d₆) δ 8.10 (d, J=7.6 Hz, 1H), 7.78 (br t, J=5.4 Hz, 1H), 7.32 (d, J=8.4 Hz, 2H), 7.20 (d, J=8.4 Hz, 2H), 6.73-5.95 (m, 2H), 4.06 (dt, J=5.6, 7.9 Hz, 1H), 3.65-3.42 (m, 32H), 3.41-3.25 (m, 1H), 3.09-2.95 (m, 2H), 2.85 (t, J=5.4 Hz, 2H), 2.57-2.51 (m, 3H), 2.43-2.29 (m, 2H), 2.04 (t, J=7.4 Hz, 2H), 1.76 (quin, J=7.6 Hz, 2H), 1.67-1.47 (m, 2H), 1.38 (s, 9H), 1.31-1.20 (m, 2H).

Intermediate A-6.9: To a solution of Intermediate A-6.8 (2.1 g, 5.6 mmol) in DMF (50 mL) was added HATU (1.7 g, 4.5 mmol) and DIEA (2.9 mL 16.7 mmol) at 25° C. followed by the addition of Intermediate A-6.7 (4.5 g, 5.6 mmol). The reaction mixture was stirred at 25° C. for 30 minutes then partitioned between H₂O (150 mL) and DCM (300 mL). The organic phase was separated, washed with a saturated aqueous solution of NaCl (50 mL×3), dried over Na₂SO₄, filtered and concentrated under reduced pressure to give a residue which was purified by flash column chromatography (SiO₂, Petroleum ether/Ethyl acetate=1/0 to 10/1) to afford Intermediate A-6.9 (11.6 g, 89% yield) as a yellow oil. MS (ESI): mass calcd. for C₆₀H₈₇ClN₄O₁₇, 1170.6; m/z found, 1171.6 [M+H]⁺. ¹H NMR (400 MHz, DMSO-d₆) δ 8.08 (br d, J=7.4 Hz, 1H), 7.89 (br d, J=7.4 Hz, 2H), 7.81 (br d, J=7.6 Hz, 1H), 7.77-7.67 (m, 2H), 7.46-7.37 (m, 2H), 7.37-7.29 (m, 3H), 7.20 (br d, J=7.8 Hz, 1H), 4.33-4.18 (m, 3H), 4.15-3.94 (m, 4H), 3.62 (s, 4H), 3.59-3.55 (m, 2H), 3.54-3.45 (m, 22H), 3.38 (br t, J=5.6 Hz, 2H), 3.22-3.13 (m, 6H), 3.05-2.92 (m, 2H), 2.56-2.51 (m, 2H), 2.43-2.27 (m, 2H), 2.17 (br t, J=7.4 Hz, 2H), 2.03 (br t, J=7.4 Hz, 2H), 1.99-1.89 (m, 1H), 1.82-1.72 (m, 2H), 1.66-1.48 (m, 2H), 1.37 (s, 9H), 1.24 (br d, J=6.0 Hz, 8H).

Intermediate A-6.10: To a solution of Intermediate A-6.9 (6.5 g, 5.6 mmol) in DCM (40 mL) was added DEA (20 mL). The reaction mixture was stirred at 25° C. for 1 hour then concentrated under reduced pressure to give a residue which was purified by prep-HPLC (column: Welch Xtimate C18 180×70 mm, 10 μm; mobile phase: [H₂O (10 mM NH₄HCO₃)-ACN]; gradient: 30%-60% B over 17.0 min) to afford Intermediate A-6.10 (3.3 g, 62% yield) as a yellow oil. MS (ESI): mass calcd. for C₄₅H₇₇ClN₄O₁₅, 948.5; m/z found, 949.6 [M+H]⁺.

Intermediate A-6.12: To a solution of Intermediate A-6.10 (6.3 g, 6.6 mmol) in DMF (70 mL) was added DIEA (2.56 g, 19.84 mmol, 3.46 mL) followed by Intermediate A-6.11 (6.6 g, 10 mmol). The mixture was stirred at 25° C. for 1 hour then concentrated under reduced pressure to give a residue. The residue was purified by prep-HPLC (column: Phenomenex luna C18 250×150 mm, 15 μm; mobile phase: [H₂O (0.1% TFA)-ACN]; gradient: 25%-55% B over 20.0 min) to afford give Intermediate A-6.12 (9.8 g, 99% yield) as a yellow oil. MS (ESI): mass calcd. for C₇₃H₁₂₇ClN₈O₂₂, 1502.9; m/z found, 1503.9 [M+H]⁺.

Intermediate A-6: To a solution of Intermediate A-6.12 (6.5 g, 4.3 mmol) in THF (22 mL) and H₂O (22 mL) was added LiOH·H₂O (816 mg, 19 mmol). The mixture was stirred at 25° C. for 10 minutes. The reaction mixture was combined with another reaction mixture of the same reaction for workup. The reaction mixture was adjusted to pH=7 by addition 1N HCl at 25° C., and then concentrated under reduced pressure. The residue was freeze-dried to afford Intermediate A-6 (8.8 g) as a white solid. MS (ESI): mass calcd. for C₇₂H₁₂₅ClN₈O₂₂, 1488.9; m/z found, 1489.9 [M+H]⁺.

### Example 2: Synthesis of Compound 8

Compound 8 was synthesized according to Scheme 2.

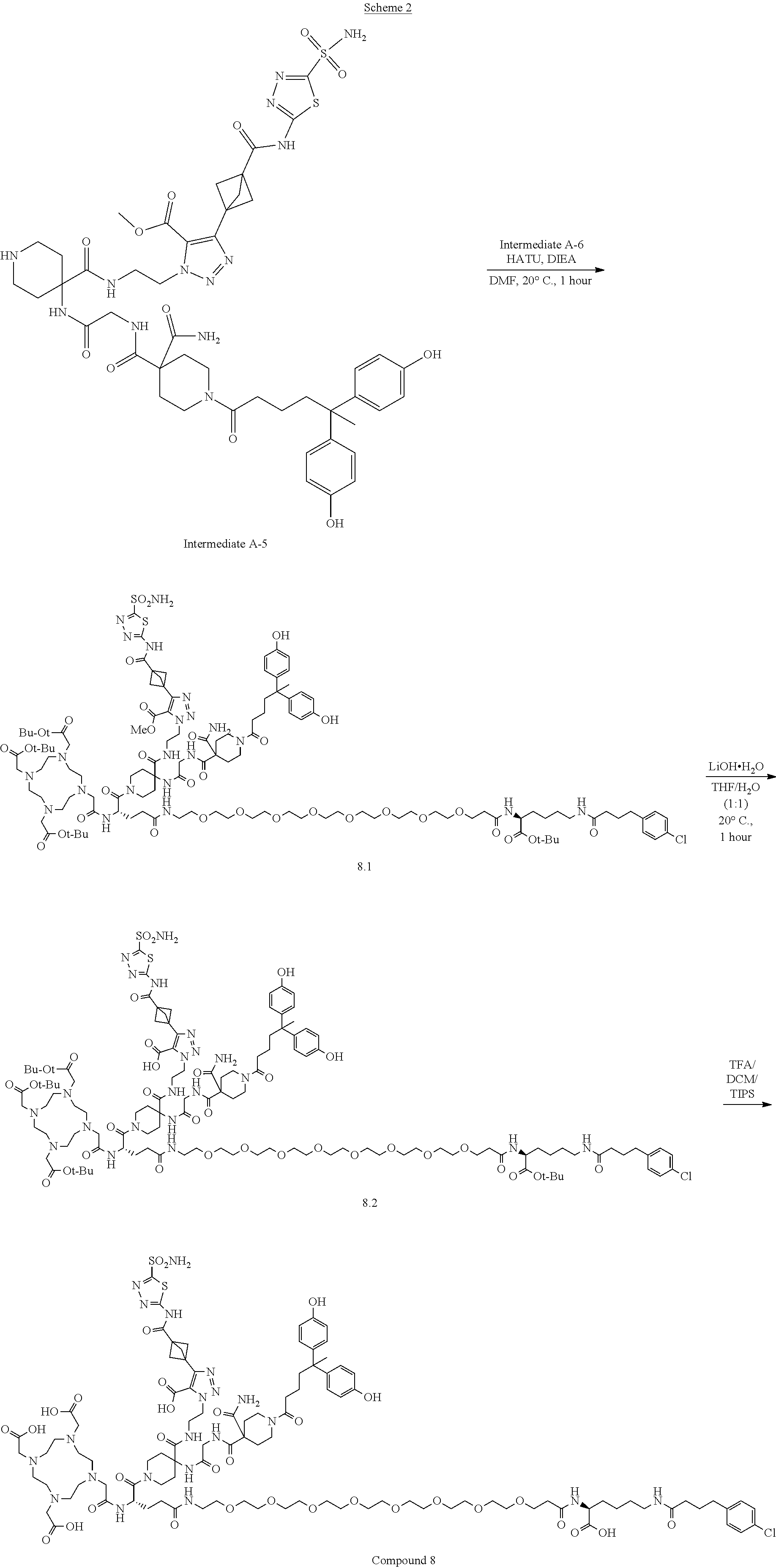

Compound 8.1: Two reactions were carried out in parallel. To a solution of Intermediate A-6 (200 mg, 0.134 mmol) in DMF (1 mL) was added DIEA (47 μL, 0.27 mmol), HATU (41 mg, 0.11 mmol) and Intermediate A-5 (143 mg, 0.134 mmol) dropwise at 0° C. The mixture was stirred at 0° C. for 30 minutes. The mixture was used for purification directly without work up. The residue was purified by prep-HPLC (TFA condition; column: Phenomenex Luna C18 100×30 mm, 5 μm; mobile phase: [H₂O (0.1% TFA)-ACN]; gradient: 32%-52% B over 9.0 min) to afford Compound 8.1 (284 mg, 42% yield) as a yellow solid. MS (ESI): mass calcd. for C₁₁₉H₁₈₂ClN₂₁O₃₃S₂, 2532.2; m/z found, 1268.4 [M/2+H]⁺. ¹H NMR (400 MHz, DMSO-d₆) δ 13.21 (d, J=4.8 Hz, 1H), 9.22-9.01 (m, 1H), 8.35 (s, 1H), 8.24-8.09 (m, 1H), 8.07 (d, J=7.6 Hz, 1H), 7.93-7.83 (m, 1H), 7.78-7.65 (m, 2H), 7.39-7.25 (m, 5H), 7.20 (d, J=8.4 Hz, 2H), 6.93 (d, J=8.6 Hz, 3H), 6.62 (d, J=8.8 Hz, 3H), 4.72-4.55 (m, 2H), 4.32-4.12 (m, 4H), 4.09-4.01 (m, 4H), 3.99-3.90 (m, 7H), 3.85-3.69 (m, 10H), 3.53-3.44 (m, 38H), 3.42-3.34 (m, 6H), 3.17 (s, 9H), 3.04-2.79 (m, 12H), 2.67 (td, J=1.8, 3.6 Hz, 2H), 2.41-2.31 (m, 4H), 2.27-2.21 (m, 1H), 2.19-2.10 (m, 2H), 2.07-1.95 (m, 6H), 1.81-1.69 (m, 4H), 1.47 (br d,)=2.6 Hz, 9H), 1.45-1.33 (m, 36H), 1.31-1.16 (m, 5H).

Compound 8.2: To a solution of Compound 8.1 (180 mg, 0.071 mmol) in MeOH (0.7 mL), THF (0.7 mL) and H₂O (0.7 mL) was added LiOH·H₂O (12 mg, 0.28 mmol) at 20° C. The mixture was stirred for 1 hour then the pH was adjusted to pH 6 by addition HCl (1N) solution. The reaction mixture was concentrated under reduced pressure to afford Compound 8.2 (180 mg) as a yellow solid. MS (ESI): mass calcd. for C₁₁₈H₁₈₀ClN₂₁O₃₃S₂, 2518.2; m/z found, 1261.1 [M/2+H]⁺.

Compound 8: To a solution of Compound 8.2 (70 mg, 0.28 mmol) in TFA (0.5 mL) and DCM (0.5 mL) was added triisopropylsilane (114 μL, 0.556 mmol) at 20° C. The reaction mixture was stirred for 16 hours and concentrated under reduced pressure to give a residue which was purified by prep-HPLC (column: Phenomenex Luna C18 100×30 mm, 5 μm; mobile phase: [H₂O (0.1% TFA)-ACN]; gradient: 20%-50% B over 8.0 min) to afford Compound 8 (11 mg, 18% yield) as a white solid. MS (ESI): mass calcd. for C₁₀₂H₁₄₈ClN₂₁O₃₃S₂, 2294.0; m/z found, 766.3 [M/3+H]⁺. ¹H NMR (400 MHz, DMSO-d₆) δ 9.17-9.07 (m, 2H), 8.35 (s, 2H), 8.20-8.04 (m, 4H), 7.75 (br t, J=5.2 Hz, 1H), 7.72-7.67 (m, 1H), 7.35-7.26 (m, 4H), 7.25-7.16 (m, 3H), 6.93 (d, J=8.6 Hz, 4H), 6.62 (d, J=8.6 Hz, 4H), 4.71-4.57 (m, 2H), 4.35-4.11 (m, 3H), 4.06-3.95 (m, 2H), 3.90-3.71 (m, 6H), 3.66-3.56 (m, 10H), 3.45-3.29 (m, 30H), 3.21-2.87 (m, 21H), 2.73-2.61 (m, 4H), 2.40-2.31 (m, 7H), 2.28-2.14 (m, 4H), 2.09-2.00 (m, 5H), 1.98-1.88 (m, 5H), 1.84-1.70 (m, 6H), 1.70-1.54 (m, 4H), 1.47 (s, 3H), 1.40-1.32 (m, 3H), 1.30-1.17 (m, 8H).

### Example 3: Synthesis of Compound 8-Lu

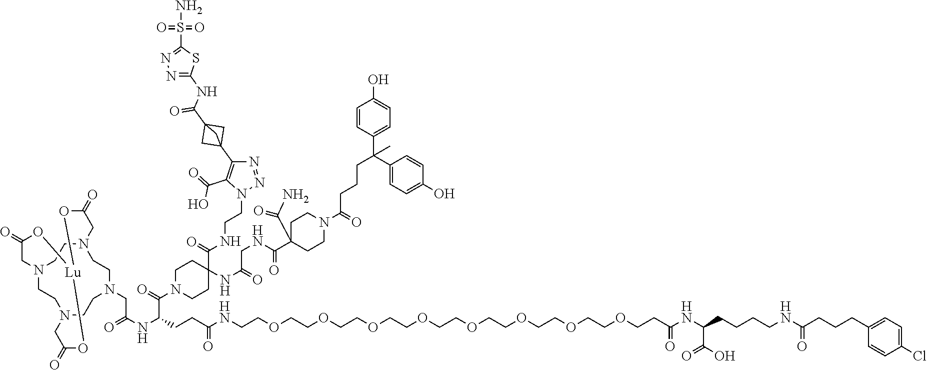

To a solution of Compound 8 (140 mg, 0.061 mmol) in H₂O (1.5 mL) was added NaOAc (0.2 M, 1.52 mL) and Lu(NO₃)₃ (46 mg, 0.12 mmol). The mixture was stirred at 60° C. for 1 hour and then concentrated under reduced pressure to give a residue. The residue was purified by prep-HPLC (column: Phenomenex Luna C18 100×30 mm, 5 μm; mobile phase: [H₂O (0.1% TFA)-ACN]; gradient: 20%-50% B over 8.0 min) to afford Compound 8-Lu (36 mg, 24% yield) as a yellow solid. MS (ESI): mass calcd. for C₁₀₂H₁₄₅ClLuN₂₁O₃₃S₂, 2465.9; m/z found, 1235.3 [M/2+H]⁺. ¹H NMR (400 MHz, DMSO-d₆) δ 9.21-9.04 (m, 2H), 8.35 (s, 2H), 8.24-8.14 (m, 1H), 8.14-7.92 (m, 3H), 7.79-7.68 (m, 2H), 7.43-7.38 (m, 1H), 7.32 (d, J=8.4 Hz, 3H), 7.20 (d, J=8.4 Hz, 2H), 6.93 (d, J=8.6 Hz, 4H), 6.62 (d, J=8.6 Hz, 4H), 4.70-4.62 (m, 2H), 4.21-4.07 (m, 1H), 3.93-3.66 (m, 7H), 3.64-3.54 (m, 7H), 3.48-3.32 (m, 29H), 3.31-3.05 (m, 10H), 3.03-2.94 (m, 4H), 2.93-2.74 (m, 5H), 2.69-2.64 (m, 2H), 2.61-2.52 (m, 13H), 2.41-2.30 (m, 7H), 2.30-2.20 (m, 4H), 2.08-1.87 (m, 10H), 1.77 (qd, J=7.4, 15.1 Hz, 5H), 1.69-1.62 (m, 2H), 1.57-1.52 (m, 1H), 1.47 (s, 3H), 1.41-1.31 (m, 3H), 1.30-1.07 (m, 6H).

### Example 3A: Synthesis of Compound 8-In

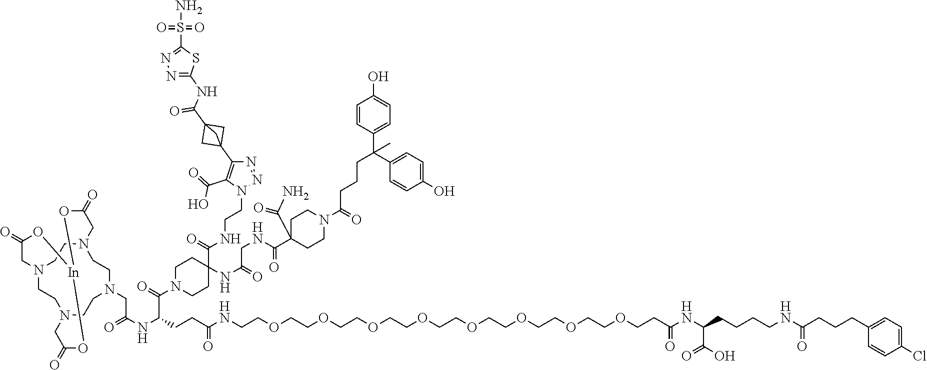

To a solution of Compound 8 (60 mg, 26 μmol) in DMF (0.5 mL) and NaOAc in H₂O (0.4 M, 1 mL) was added InCl₃ (9 mg, 39 μmol, 2.5 μL). The reaction mixture was stirred at 80° C. for 1 hour. The reaction mixture was concentrated under reduced pressure to give a residue. The residue was purified by prep-HPLC (column: Waters Xbridge BEH C18 100*30 mm*10 μm; mobile phase: [H₂O (0.5% CH₃COOH)-ACN (0.5% CH₃COOH)]; gradient: 10%-60% B over 15.0 min) to give Compound 8-In (31 mg, 49% yield) as a white solid. MS (ESI): m/z found, 1204.5 [M/2+H]⁺.

### Example 3B: Synthesis of Compound 8-Tb

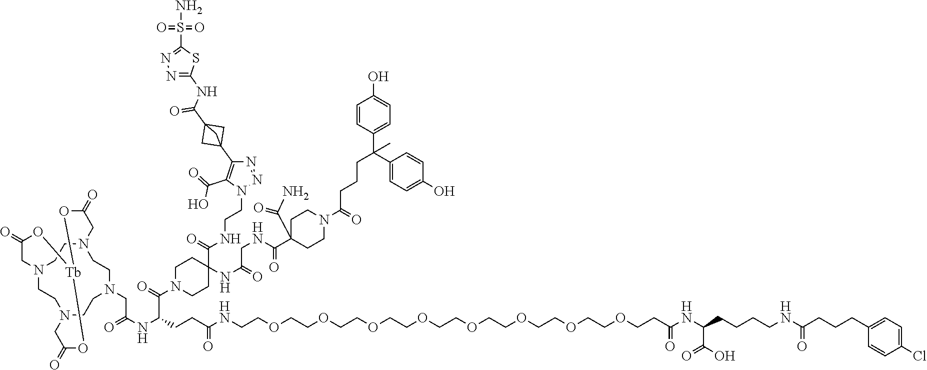

To a solution of Compound 8 (5 mg, 2.2 μmol) in 0.4M NaOAc buffer pH=5 (700 μL) was added TbCl₃ (1.2 mg, 4.6 μmol). The reaction mixture was stirred at 60° C. for 1 hour and then purified by prep-HPLC (column: Luna, 30×100 mm, 10-80% ACN/H₂O+0.1% TFA) to give Compound 8-Tb (3 mg, 56% yield) as a white solid. MS (ESI): m/z found, 1226.5 [M/2+H]⁺.

### Example 3C: Other Radiolabeled Complexes can Also be Prepared Using Methods Analogous to Those Described Herein

General protocol for cold labeling of compounds with Pb: To a solution of the appropriate empty-chelator compound (1 equiv.) in NaOAc in H₂O (0.2M) was added Pb(OAc)₂ (1.5 equiv.). The reaction mixture was stirred at 45° C. for about 1 hour and then concentrated under reduced pressure to give a residue. The residue was purified by preparative HPLC to afford the corresponding cold metal labeled compound.

General protocol for cold labeling of compounds with Cu: To a solution of the appropriate empty-chelator compound (1 equiv.) in NH₄OAc in H₂O (0.1M) was added CuCl₂ (10 equiv.). The reaction mixture was stirred at 60° C. for about 1 hour and then concentrated under reduced pressure to give a residue. The residue was purified by preparative HPLC to afford the corresponding cold metal labeled compound.

### Example 4: CA-IX Compounds

Provided herein are compounds as described in Table 1 and Table 2 below. Such Compounds were synthesized according to the methods described herein and the mass spectrometry analysis performed after synthesis of each compound is provided in Table 3.

TABLE 1
Structure
Compound 1:

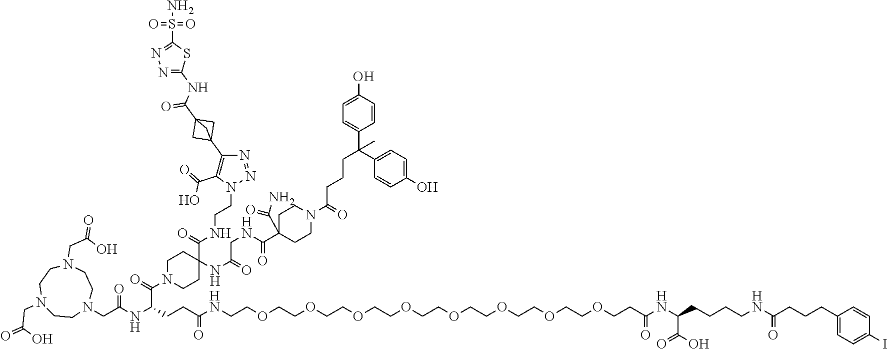 [SMILES: CC(CCCC(=O)N1CCC(C(N)=O)(C(=O)NCC(=O)NC2(C(=O)NCCn3nnc(C45CC(C(=O)Nc6nnc(S(N)(=O)=O)s6)(C4)C5)c3C(=O)O)CCN(C(=O)[C@H](CCC(=O)NCCOCCOCCOCCOCCOCCOCCOCCOCCC(=O)N[C@@H](CCCCNC(=O)CCCc3ccc(I)cc3)C(=O)O)NC(=O)CN3CCN(CC(=O)O)CCN(CC(=O)O)CC3)CC2)CC1)(c1ccc(O)cc1)c1ccc(O)cc1]

Compound 1-Cu:

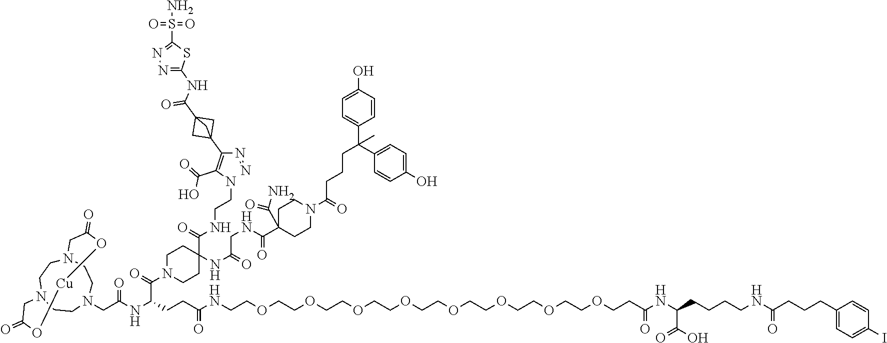

Compound 2:

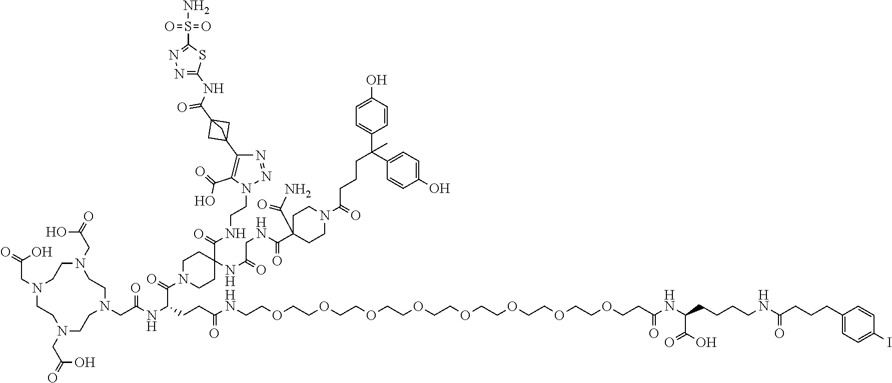 [SMILES: CC(CCCC(=O)N1CCC(C(N)=O)(C(=O)NCC(=O)NC2(C(=O)NCCn3nnc(C45CC(C(=O)Nc6nnc(S(N)(=O)=O)s6)(C4)C5)c3C(=O)O)CCN(C(=O)[C@H](CCC(=O)NCCOCCOCCOCCOCCOCCOCCOCCOCCC(=O)N[C@@H](CCCCNC(=O)CCCc3ccc(I)cc3)C(=O)O)NC(=O)CN3CCN(CC(=O)O)CCN(CC(=O)O)CCN(CC(=O)O)CC3)CC2)CC1)(c1ccc(O)cc1)c1ccc(O)cc1]

Compound 2-Lu:

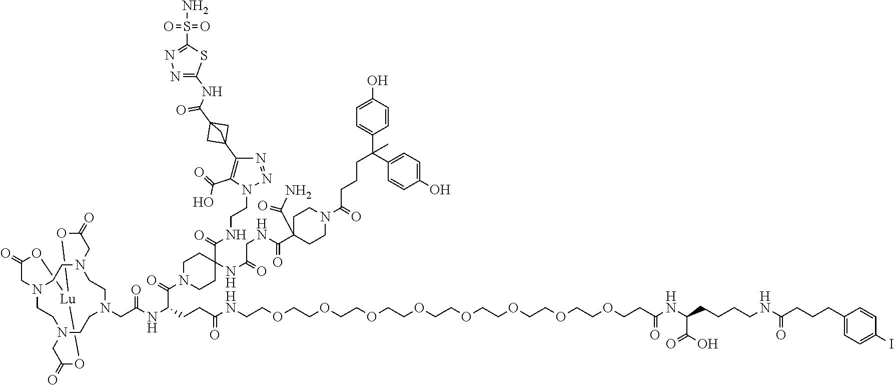

Compound 3:

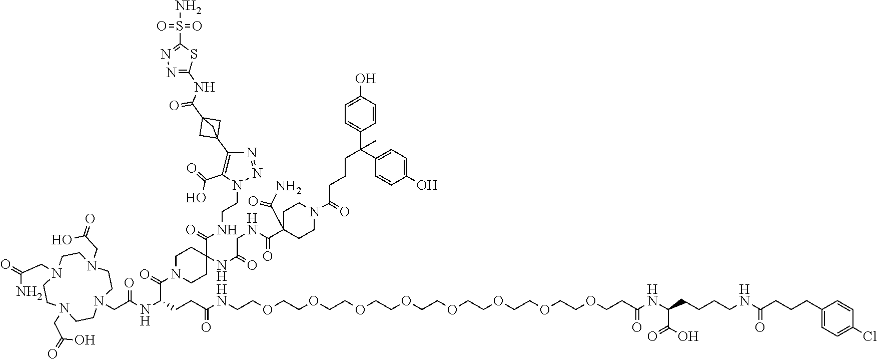 [SMILES: CC(CCCC(=O)N1CCC(C(N)=O)(C(=O)NCC(=O)NC2(C(=O)NCCn3nnc(C45CC(C(=O)Nc6nnc(S(N)(=O)=O)s6)(C4)C5)c3C(=O)O)CCN(C(=O)[C@H](CCC(=O)NCCOCCOCCOCCOCCOCCOCCOCCOCCC(=O)N[C@@H](CCCCNC(=O)CCCc3ccc(Cl)cc3)C(=O)O)NC(=O)CN3CCN(CC(=O)O)CCN(CC(N)=O)CCN(CC(=O)O)CC3)CC2)CC1)(c1ccc(O)cc1)c1ccc(O)cc1]

Compound 3-Pb:

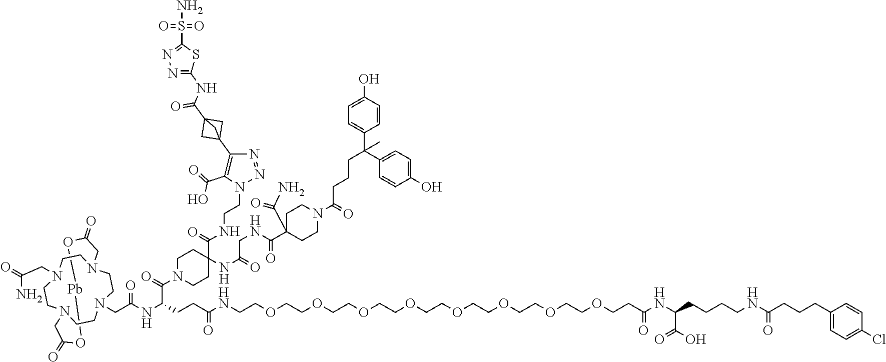

Compound 4:

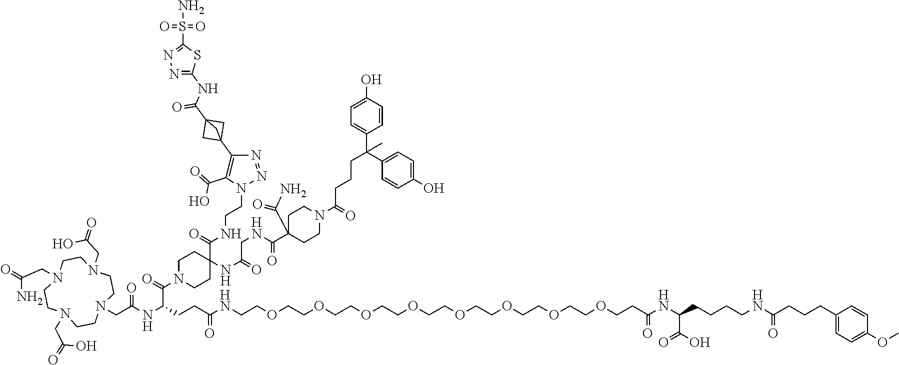 [SMILES: COc1ccc(CCCC(=O)NCCCC[C@H](NC(=O)CCOCCOCCOCCOCCOCCOCCOCCOCCNC(=O)CC[C@H](NC(=O)CN2CCN(CC(=O)O)CCN(CC(N)=O)CCN(CC(=O)O)CC2)C(=O)N2CCC(NC(=O)CNC(=O)C3(C(N)=O)CCN(C(=O)CCCC(C)(c4ccc(O)cc4)c4ccc(O)cc4)CC3)(C(=O)NCCn3nnc(C45CC(C(=O)Nc6nnc(S(N)(=O)=O)s6)(C4)C5)c3C(=O)O)CC2)C(=O)O)cc1]

Compound 4-Pb:

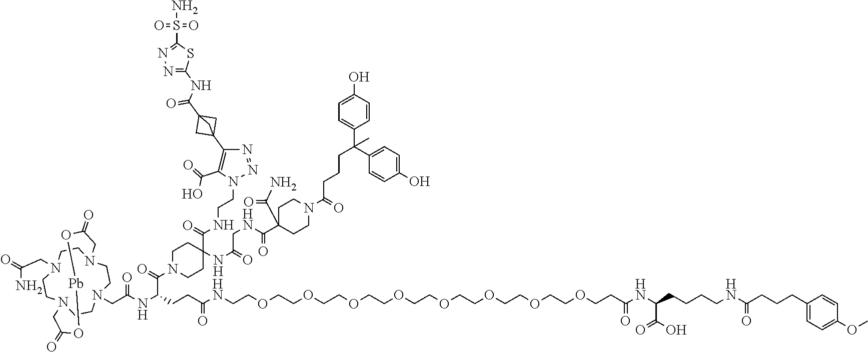

Compound 5:

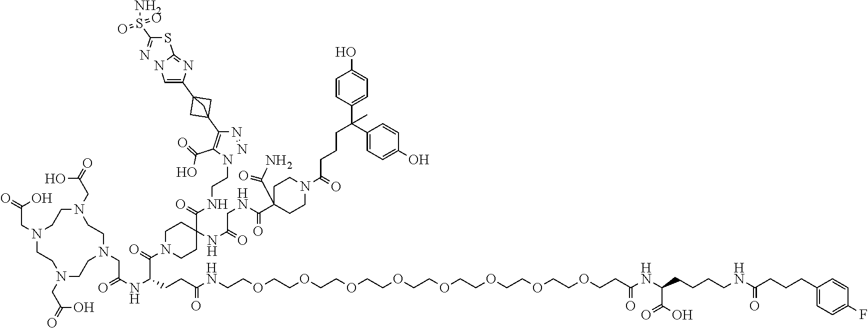 [SMILES: CC(CCCC(=O)N1CCC(C(N)=O)(C(=O)NCC(=O)NC2(C(=O)NCCn3nnc(C45CC(c6cn7nc(S(N)(=O)=O)sc7n6)(C4)C5)c3C(=O)O)CCN(C(=O)[C@H](CCC(=O)NCCOCCOCCOCCOCCOCCOCCOCCOCCC(=O)N[C@@H](CCCCNC(=O)CCCc3ccc(F)cc3)C(=O)O)NC(=O)CN3CCN(CC(=O)O)CCN(CC(=O)O)CCN(CC(=O)O)CC3)CC2)CC1)(c1ccc(O)cc1)c1ccc(O)cc1]

Compound 5-Lu:

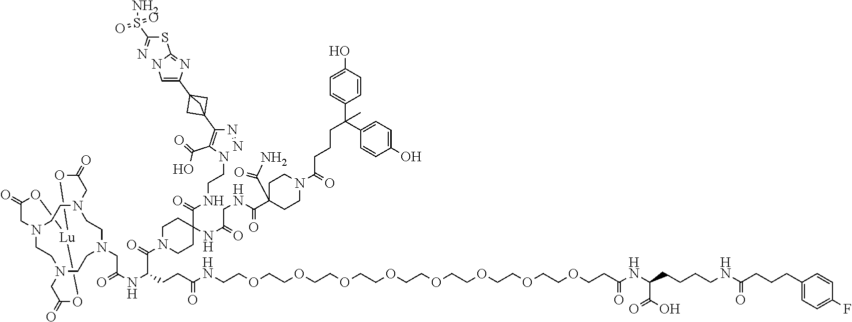

Compound 6:

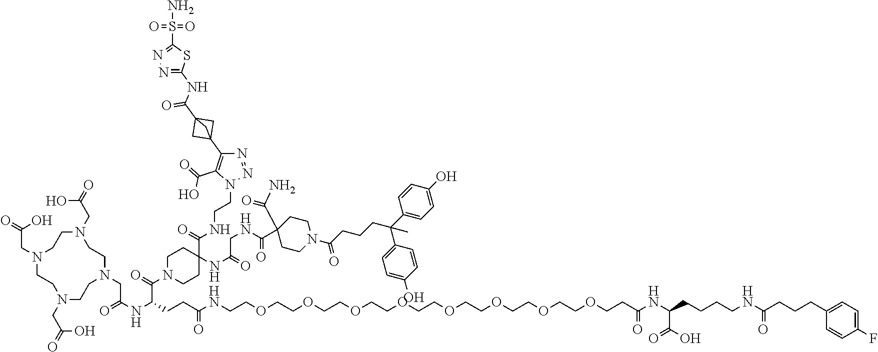 [SMILES: CC(CCCC(=O)N1CCC(C(N)=O)(C(=O)NCC(=O)NC2(C(=O)NCCn3nnc(C45CC(C(=O)Nc6nnc(S(N)(=O)=O)s6)(C4)C5)c3C(=O)O)CCN(C(=O)[C@H](CCC(=O)NCCOCCOCCOCCOCCOCCOCCOCCOCCC(=O)N[C@@H](CCCCNC(=O)CCCc3ccc(F)cc3)C(=O)O)NC(=O)CN3CCN(CC(=O)O)CCN(CC(=O)O)CCN(CC(=O)O)CC3)CC2)CC1)(c1ccc(O)cc1)c1ccc(O)cc1]

Compound 6-Lu:

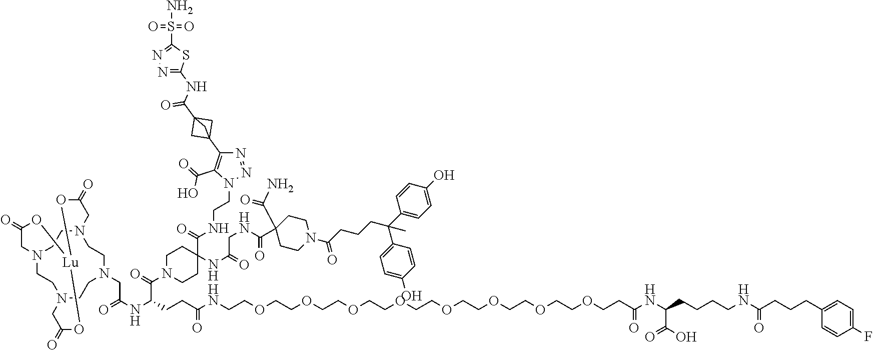

Compound 7:

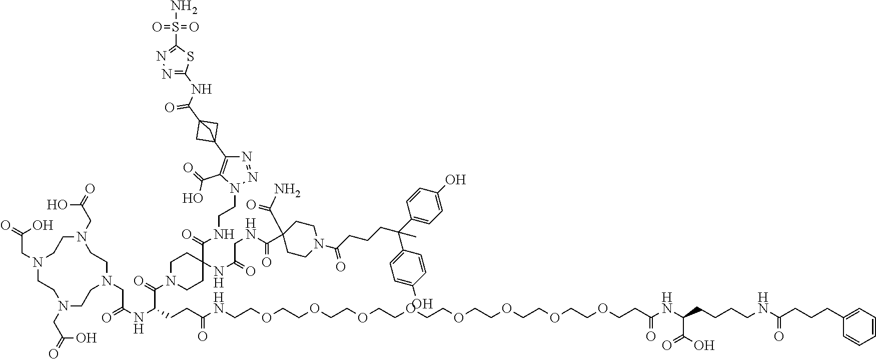 [SMILES: CC(CCCC(=O)N1CCC(C(N)=O)(C(=O)NCC(=O)NC2(C(=O)NCCn3nnc(C45CC(C(=O)Nc6nnc(S(N)(=O)=O)s6)(C4)C5)c3C(=O)O)CCN(C(=O)[C@H](CCC(=O)NCCOCCOCCOCCOCCOCCOCCOCCOCCC(=O)N[C@@H](CCCCNC(=O)CCCc3ccccc3)C(=O)O)NC(=O)CN3CCN(CC(=O)O)CCN(CC(=O)O)CCN(CC(=O)O)CC3)CC2)CC1)(c1ccc(O)cc1)c1ccc(O)cc1]

Compound 7-Lu:

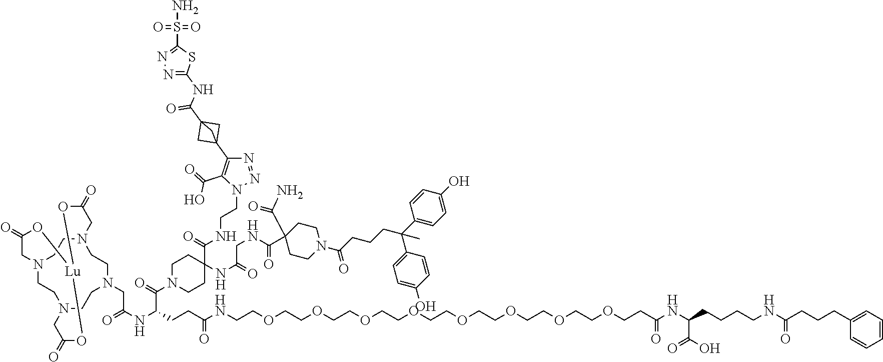

Compound 8:

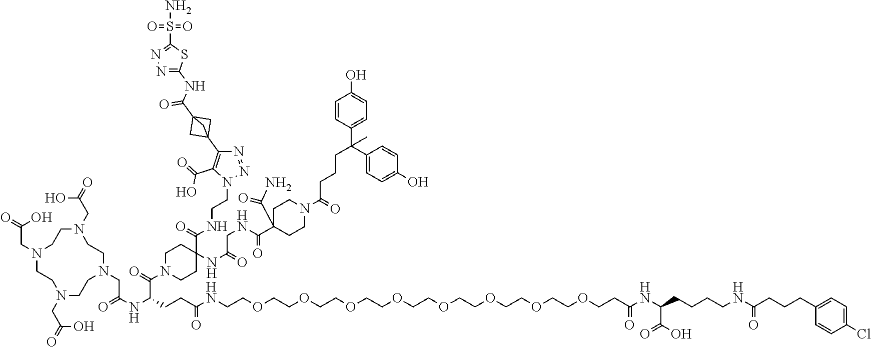 [SMILES: CC(CCCC(=O)N1CCC(C(N)=O)(C(=O)NCC(=O)NC2(C(=O)NCCn3nnc(C45CC(C(=O)Nc6nnc(S(N)(=O)=O)s6)(C4)C5)c3C(=O)O)CCN(C(=O)[C@H](CCC(=O)NCCOCCOCCOCCOCCOCCOCCOCCOCCC(=O)N[C@@H](CCCCNC(=O)CCCc3ccc(Cl)cc3)C(=O)O)NC(=O)CN3CCN(CC(=O)O)CCN(CC(=O)O)CCN(CC(=O)O)CC3)CC2)CC1)(c1ccc(O)cc1)c1ccc(O)cc1]

Compound 8-Lu:

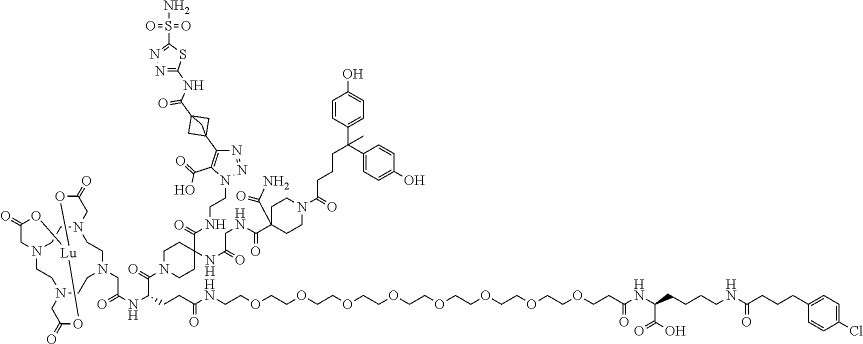

Compound 8-In

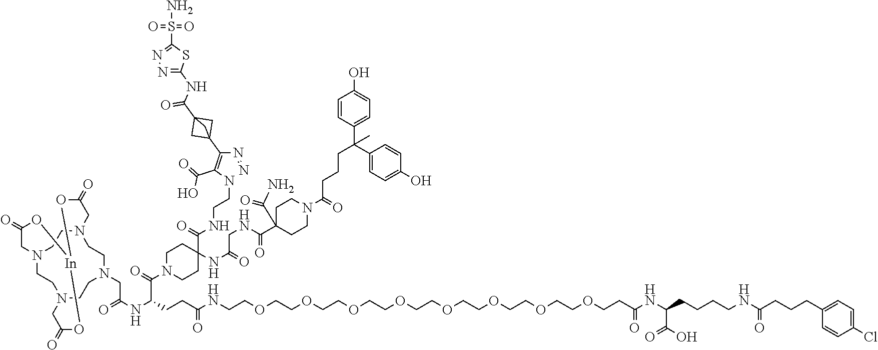

Compound 8-Tb

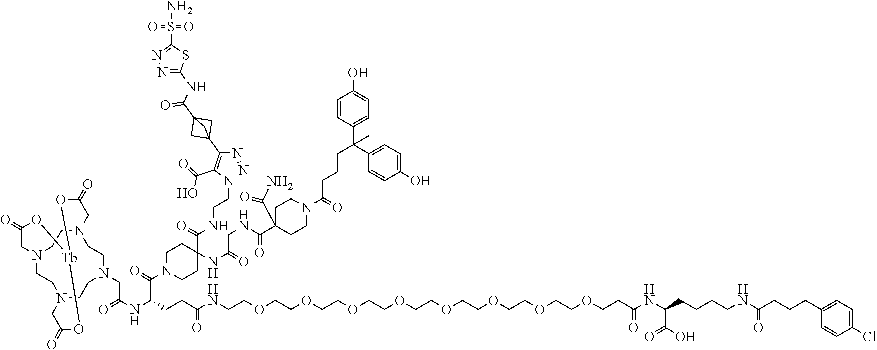

Compound 9:

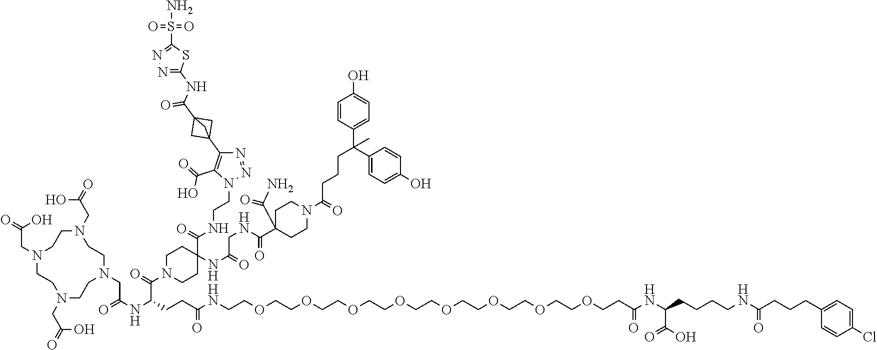 [SMILES: CC(CCCC(=O)N1CCC(C(N)=O)(C(=O)NCC(=O)NC2(C(=O)NCCn3nnc(C45CC(C(=O)Nc6nnc(S(N)(=O)=O)s6)(C4)C5)c3C(=O)O)CCN(C(=O)[C@H](CCC(=O)NCCOCCOCCOCCOCCOCCOCCOCCOCCC(=O)N[C@@H](CCCCNC(=O)CCCc3ccc(Cl)cc3)C(=O)O)NC(=O)CN3CCN(CC(=O)O)CCN(CC(=O)O)CCN(CC(=O)O)CC3)CC2)CC1)(c1ccc(O)cc1)c1ccc(O)cc1]

Compound 9-Lu:

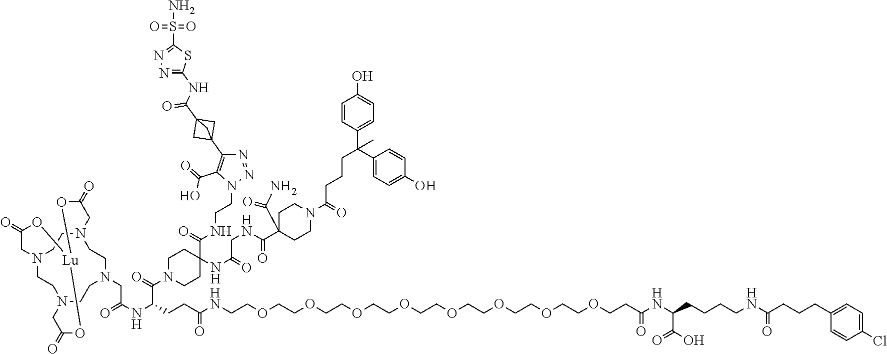

Compound 10:

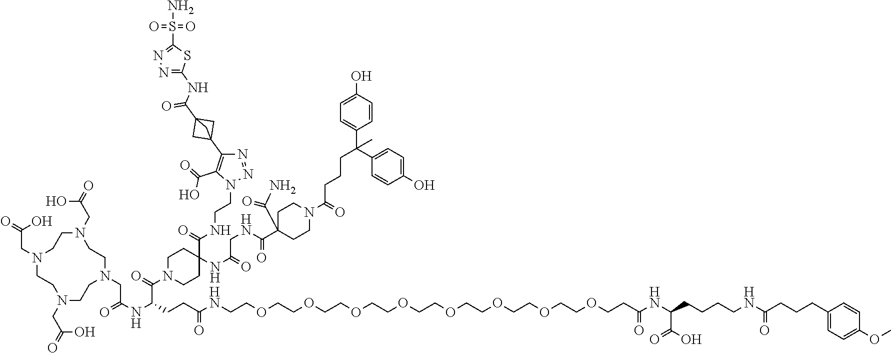 [SMILES: COc1ccc(CCCC(=O)NCCCC[C@H](NC(=O)CCOCCOCCOCCOCCOCCOCCOCCOCCNC(=O)CC[C@H](NC(=O)CN2CCN(CC(=O)O)CCN(CC(=O)O)CCN(CC(=O)O)CC2)C(=O)N2CCC(NC(=O)CNC(=O)C3(C(N)=O)CCN(C(=O)CCCC(C)(c4ccc(O)cc4)c4ccc(O)cc4)CC3)(C(=O)NCCn3nnc(C45CC(C(=O)Nc6nnc(S(N)(=O)=O)s6)(C4)C5)c3C(=O)O)CC2)C(=O)O)cc1]

Compound 10-Lu:

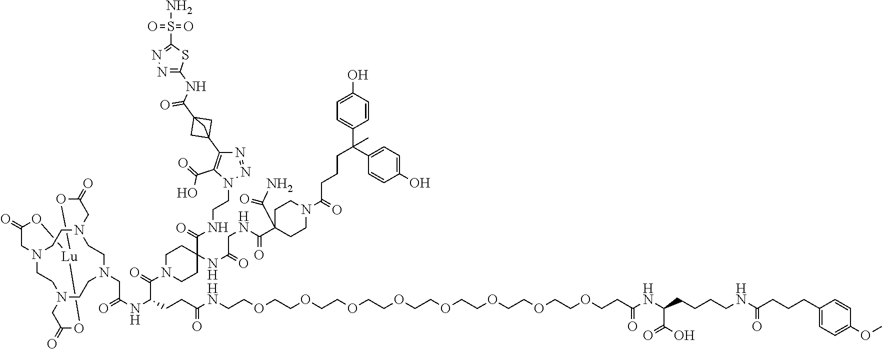

Compound 11:

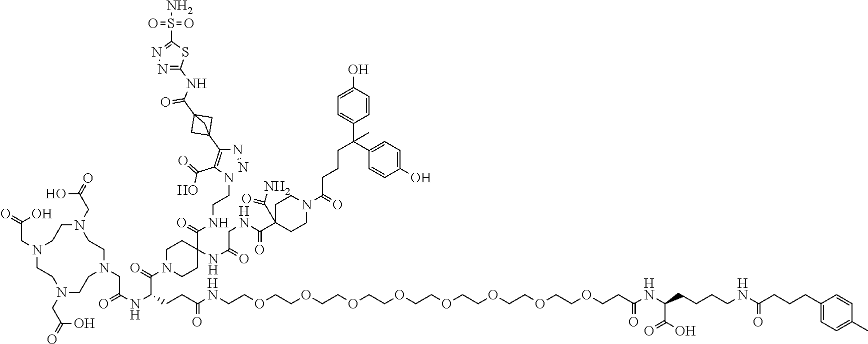 [SMILES: Cc1ccc(CCCC(=O)NCCCC[C@H](NC(=O)CCOCCOCCOCCOCCOCCOCCOCCOCCNC(=O)CC[C@H](NC(=O)CN2CCN(CC(=O)O)CCN(CC(=O)O)CCN(CC(=O)O)CC2)C(=O)N2CCC(NC(=O)CNC(=O)C3(C(N)=O)CCN(C(=O)CCCC(C)(c4ccc(O)cc4)c4ccc(O)cc4)CC3)(C(=O)NCCn3nnc(C45CC(C(=O)Nc6nnc(S(N)(=O)=O)s6)(C4)C5)c3C(=O)O)CC2)C(=O)O)cc1]

Compound 11-Lu:

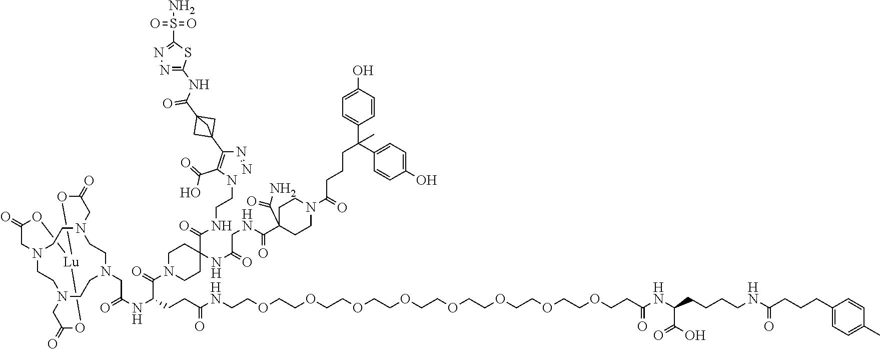

Compound 12:

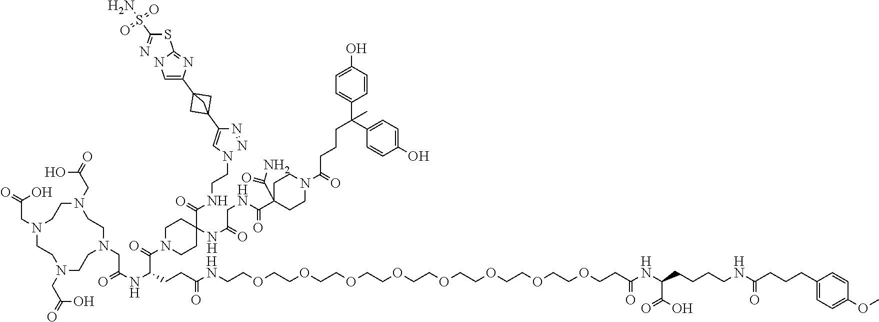 [SMILES: COc1ccc(CCCC(=O)NCCCC[C@H](NC(=O)CCOCCOCCOCCOCCOCCOCCOCCOCCNC(=O)CC[C@H](NC(=O)CN2CCN(CC(=O)O)CCN(CC(=O)O)CCN(CC(=O)O)CC2)C(=O)N2CCC(NC(=O)CNC(=O)C3(C(N)=O)CCN(C(=O)CCCC(C)(c4ccc(O)cc4)c4ccc(O)cc4)CC3)(C(=O)NCCn3cc(C45CC(c6cn7nc(S(N)(=O)=O)sc7n6)(C4)C5)nn3)CC2)C(=O)O)cc1]

Compound 12-Lu:

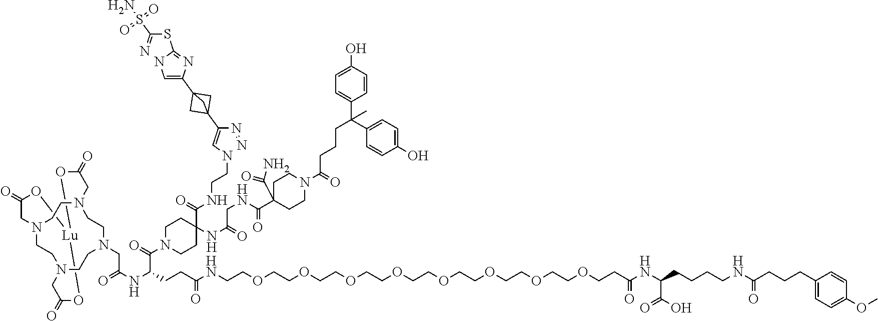

Compound 13:

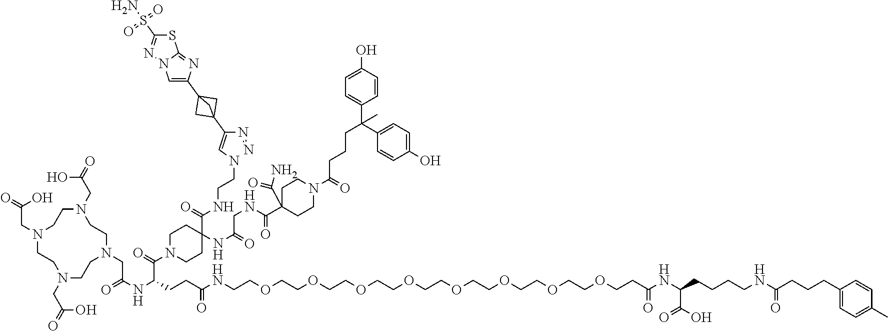 [SMILES: Cc1ccc(CCCC(=O)NCCCC[C@H](NC(=O)CCOCCOCCOCCOCCOCCOCCOCCOCCNC(=O)CC[C@H](NC(=O)CN2CCN(CC(=O)O)CCN(CC(=O)O)CCN(CC(=O)O)CC2)C(=O)N2CCC(NC(=O)CNC(=O)C3(C(N)=O)CCN(C(=O)CCCC(C)(c4ccc(O)cc4)c4ccc(O)cc4)CC3)(C(=O)NCCn3cc(C45CC(c6cn7nc(S(N)(=O)=O)sc7n6)(C4)C5)nn3)CC2)C(=O)O)cc1]

Compound 13-Lu:

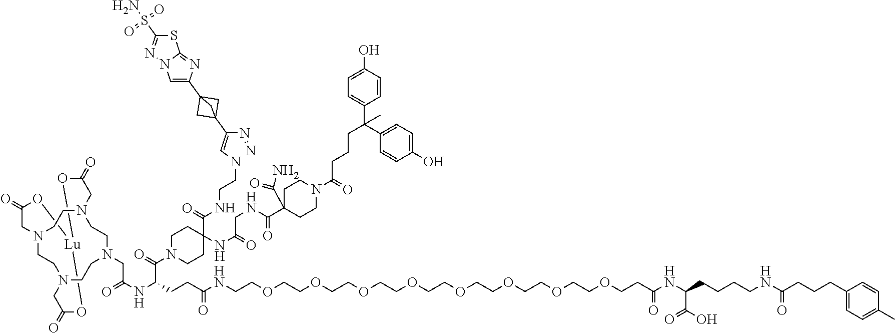

Compound 14:

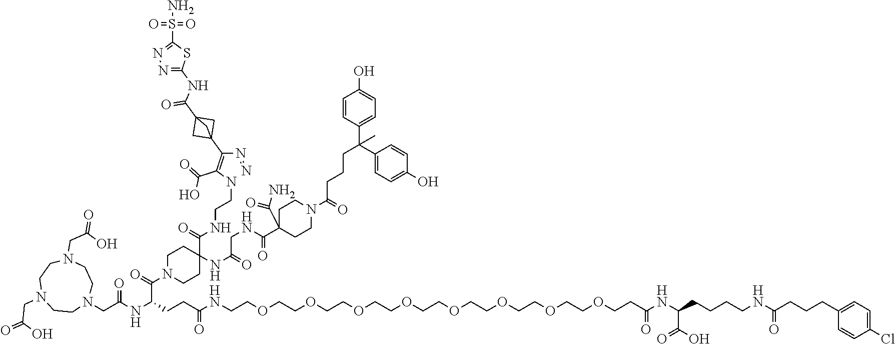 [SMILES: CC(CCCC(=O)N1CCC(C(N)=O)(C(=O)NCC(=O)NC2(C(=O)NCCn3nnc(C45CC(C(=O)Nc6nnc(S(N)(=O)=O)s6)(C4)C5)c3C(=O)O)CCN(C(=O)[C@H](CCC(=O)NCCOCCOCCOCCOCCOCCOCCOCCOCCC(=O)N[C@@H](CCCCNC(=O)CCCc3ccc(Cl)cc3)C(=O)O)NC(=O)CN3CCN(CC(=O)O)CCN(CC(=O)O)CC3)CC2)CC1)(c1ccc(O)cc1)c1ccc(O)cc1]

Compound 14-Cu:

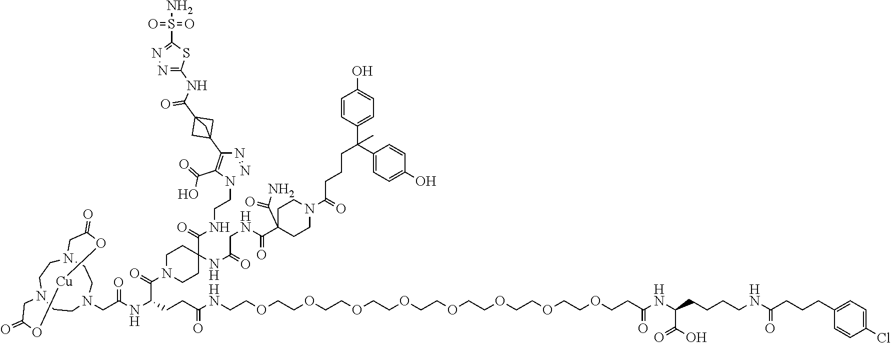

Compound 15:

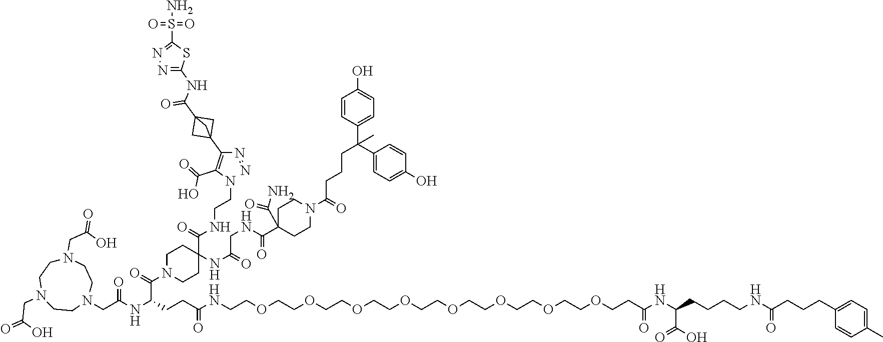 [SMILES: Cc1ccc(CCCC(=O)NCCCC[C@H](NC(=O)CCOCCOCCOCCOCCOCCOCCOCCOCCNC(=O)CC[C@H](NC(=O)CN2CCN(CC(=O)O)CCN(CC(=O)O)CC2)C(=O)N2CCC(NC(=O)CNC(=O)C3(C(N)=O)CCN(C(=O)CCCC(C)(c4ccc(O)cc4)c4ccc(O)cc4)CC3)(C(=O)NCCn3nnc(C45CC(C(=O)Nc6nnc(S(N)(=O)=O)s6)(C4)C5)c3C(=O)O)CC2)C(=O)O)cc1]

Compound 15-Cu:

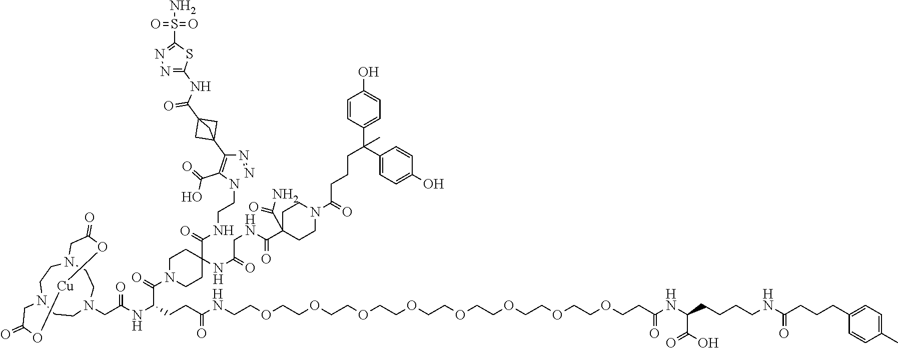

Compound 16:

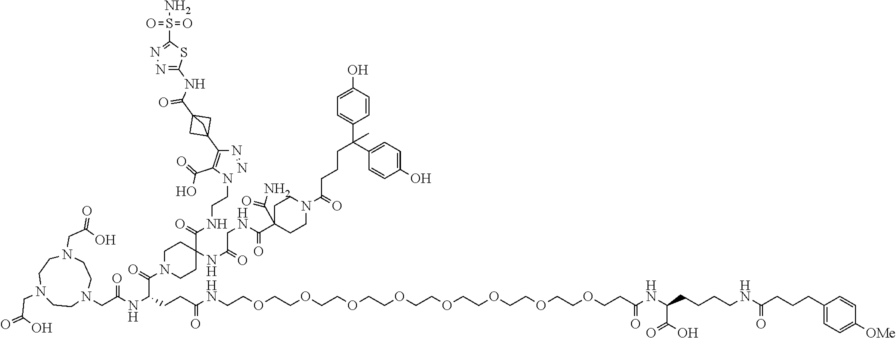 [SMILES: COc1ccc(CCCC(=O)NCCCC[C@H](NC(=O)CCOCCOCCOCCOCCOCCOCCOCCOCCNC(=O)CC[C@H](NC(=O)CN2CCN(CC(=O)O)CCN(CC(=O)O)CC2)C(=O)N2CCC(NC(=O)CNC(=O)C3(C(N)=O)CCN(C(=O)CCCC(C)(c4ccc(O)cc4)c4ccc(O)cc4)CC3)(C(=O)NCCn3nnc(C45CC(C(=O)Nc6nnc(S(N)(=O)=O)s6)(C4)C5)c3C(=O)O)CC2)C(=O)O)cc1]

Compound 16-Cu:

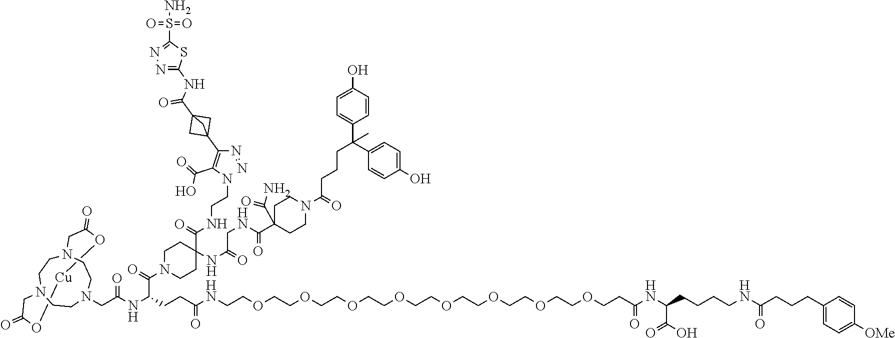

Compound 26-Ph

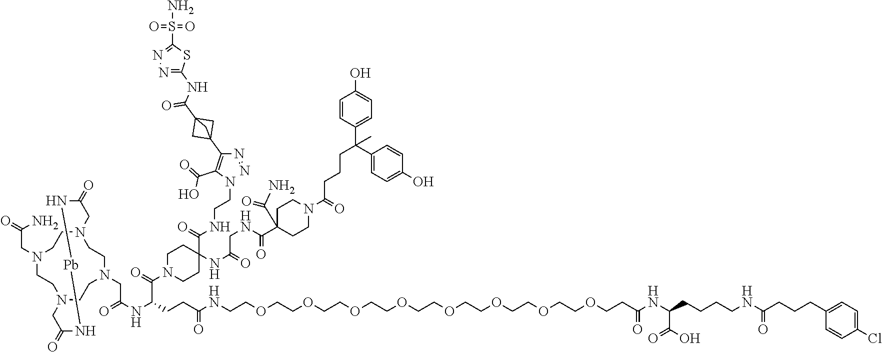

TABLE 2
Structure
Compound 17:

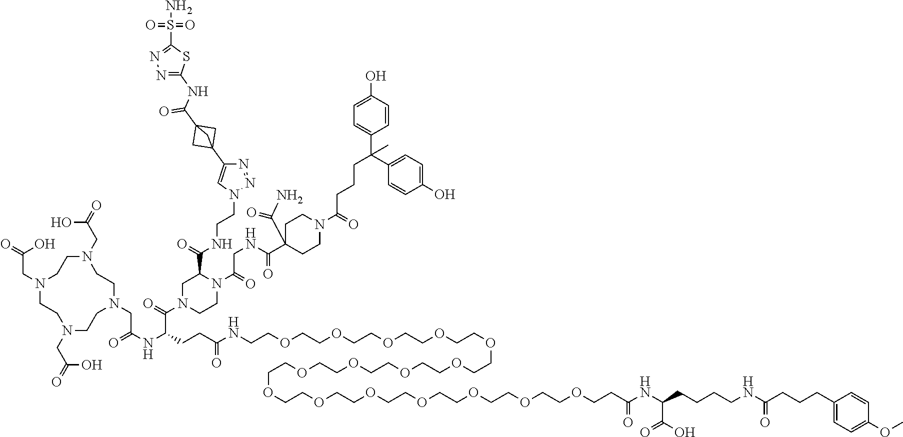 [SMILES: COc1ccc(CCCC(=O)NCCCC[C@H](NC(=O)CCOCCOCCOCCOCCOCCOCCOCCOCCOCCOCCOCCOCCOCCOCCOCCOCCNC(=O)CC[C@H](NC(=O)CN2CCN(CC(=O)O)CCN(CC(=O)O)CCN(CC(=O)O)CC2)C(=O)N2CCN(C(=O)CNC(=O)C3(C(N)=O)CCN(C(=O)CCCC(C)(c4ccc(O)cc4)c4ccc(O)cc4)CC3)[C@H](C(=O)NCCn3cc(C45CC(C(=O)Nc6nnc(S(N)(=O)=O)s6)(C4)C5)nn3)C2)C(=O)O)cc1]

Compound 17-Lu:

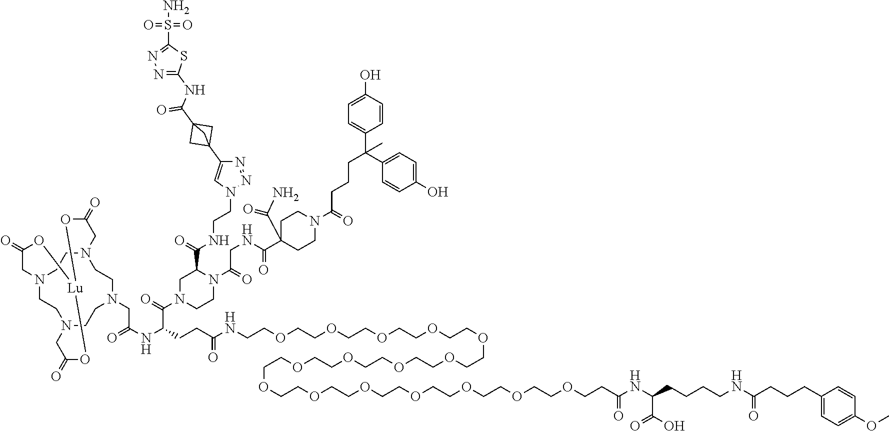

Compound 18:

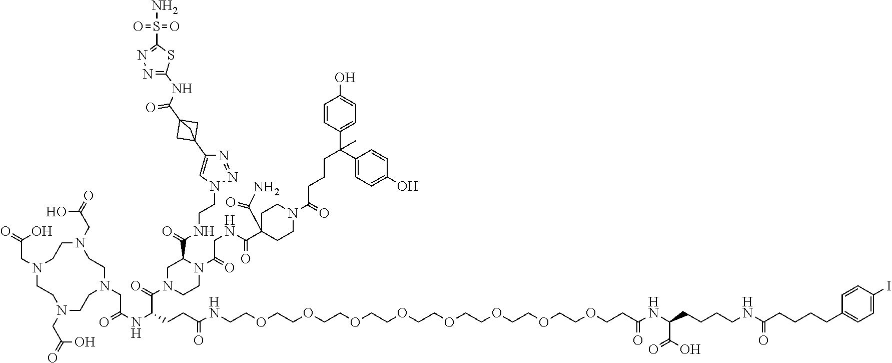 [SMILES: CC(CCCC(=O)N1CCC(C(N)=O)(C(=O)NCC(=O)N2CCN(C(=O)[C@H](CCC(=O)NCCOCCOCCOCCOCCOCCOCCOCCOCCC(=O)N[C@@H](CCCCNC(=O)CCCCc3ccc(I)cc3)C(=O)O)NC(=O)CN3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)CC1)(c1ccc(O)cc1)c1ccc(O)cc1]

Compound 18-Lu:

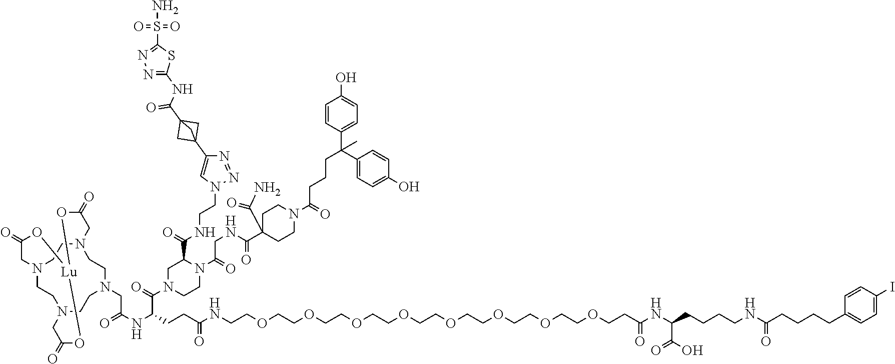

Compound 19:

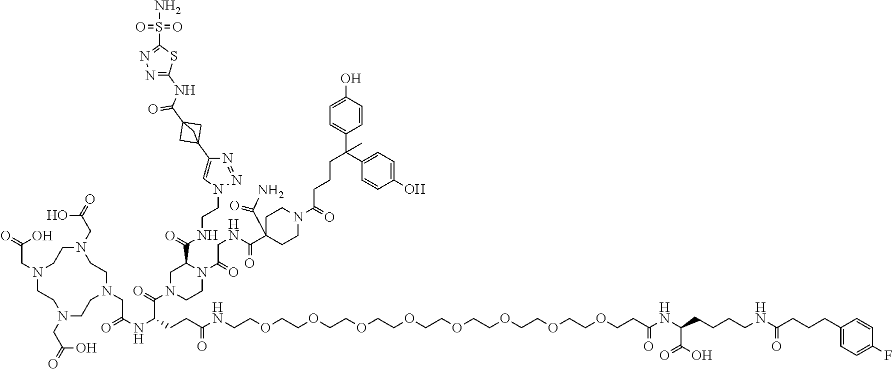 [SMILES: CC(CCCC(=O)N1CCC(C(N)=O)(C(=O)NCC(=O)N2CCN(C(=O)[C@H](CCC(=O)NCCOCCOCCOCCOCCOCCOCCOCCOCCC(=O)N[C@@H](CCCCNC(=O)CCCc3ccc(F)cc3)C(=O)O)NC(=O)CN3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)CC1)(c1ccc(O)cc1)c1ccc(O)cc1]

Compound 19-Lu:

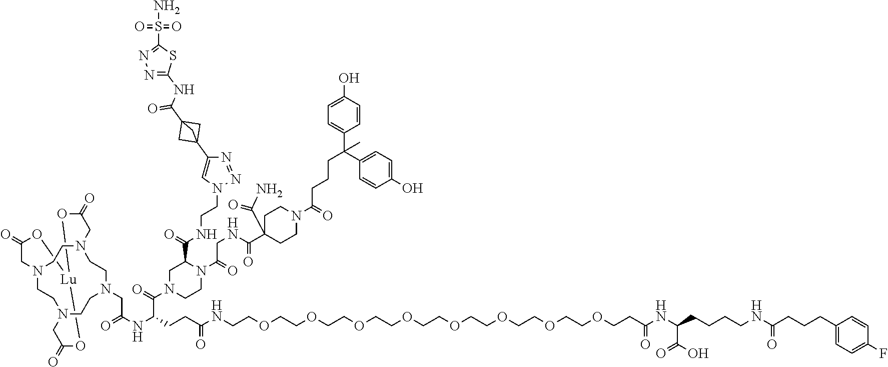

Compound 20:

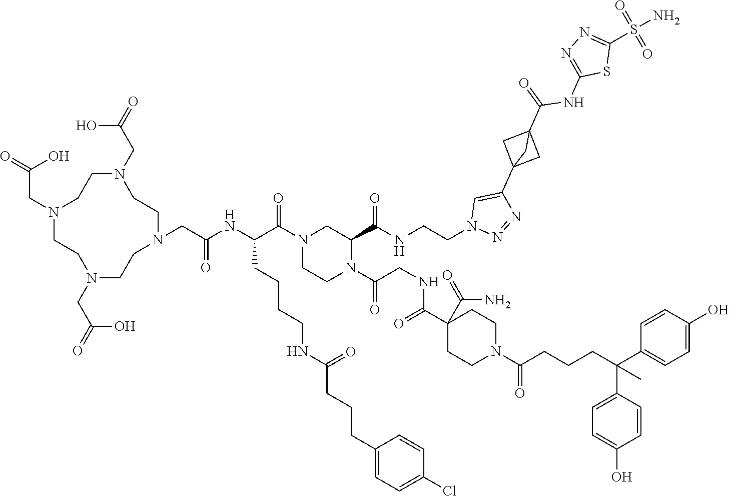 [SMILES: CC(CCCC(=O)N1CCC(C(N)=O)(C(=O)NCC(=O)N2CCN(C(=O)[C@H](CCCCNC(=O)CCCc3ccc(Cl)cc3)NC(=O)CN3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)CC1)(c1ccc(O)cc1)c1ccc(O)cc1]

Compound 20-Lu:

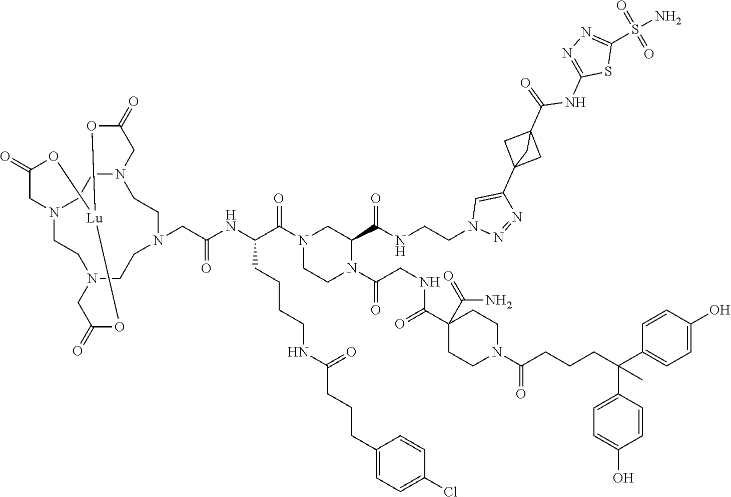

Compound 21:

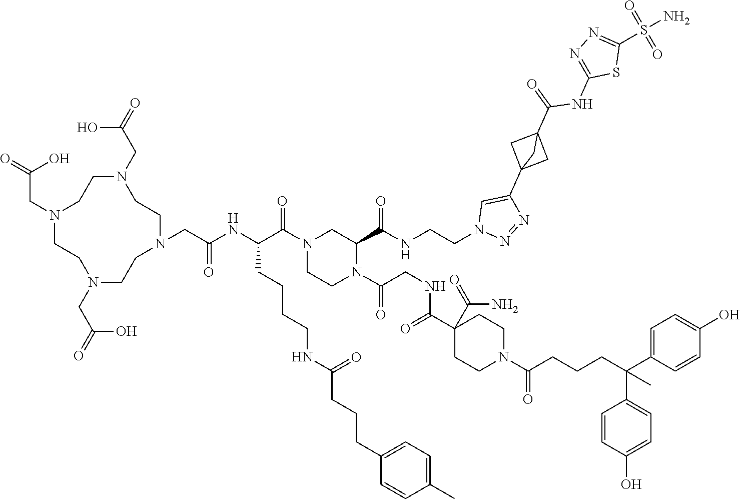 [SMILES: Cc1ccc(CCCC(=O)NCCCC[C@H](NC(=O)CN2CCN(CC(=O)O)CCN(CC(=O)O)CCN(CC(=O)O)CC2)C(=O)N2CCN(C(=O)CNC(=O)C3(C(N)=O)CCN(C(=O)CCCC(C)(c4ccc(O)cc4)c4ccc(O)cc4)CC3)[C@H](C(=O)NCCn3cc(C45CC(C(=O)Nc6nnc(S(N)(=O)=O)s6)(C4)C5)nn3)C2)cc1]

Compound 21-Lu

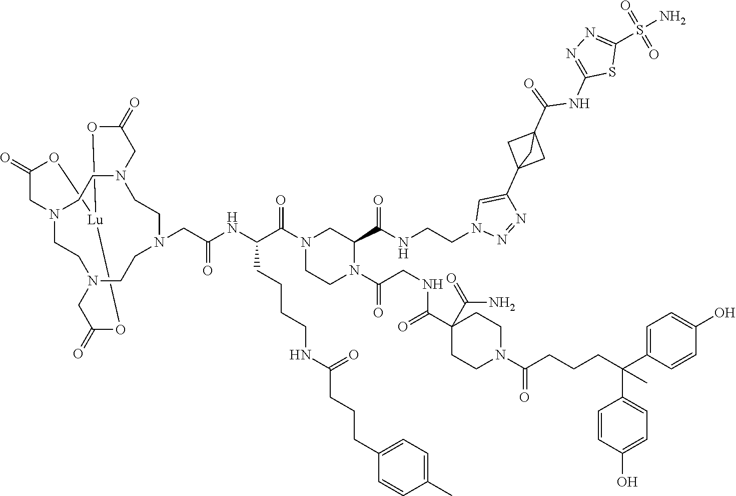

Compound 22:

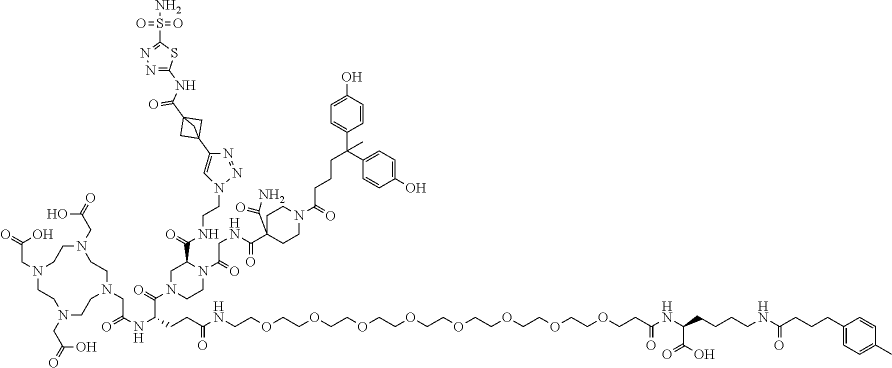 [SMILES: Cc1ccc(CCCC(=O)NCCCC[C@H](NC(=O)CCOCCOCCOCCOCCOCCOCCOCCOCCNC(=O)CC[C@H](NC(=O)CN2CCN(CC(=O)O)CCN(CC(=O)O)CCN(CC(=O)O)CC2)C(=O)N2CCN(C(=O)CNC(=O)C3(C(N)=O)CCN(C(=O)CCCC(C)(c4ccc(O)cc4)c4ccc(O)cc4)CC3)[C@H](C(=O)NCCn3cc(C45CC(C(=O)Nc6nnc(S(N)(=O)=O)s6)(C4)C5)nn3)C2)C(=O)O)cc1]

Compound 22-Lu:

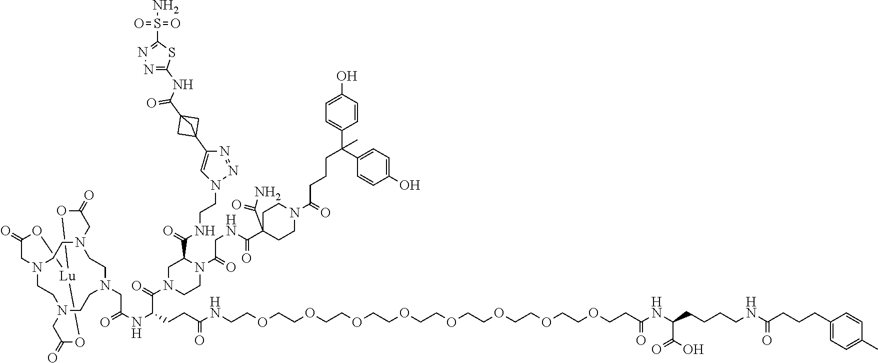

Compound 23:

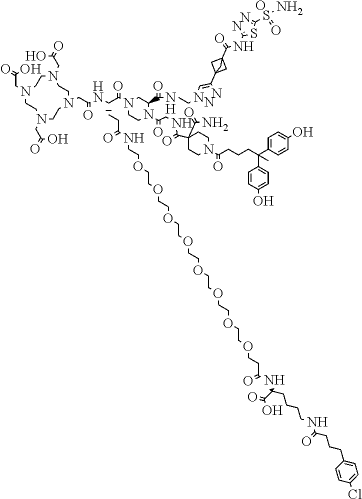 [SMILES: CC(CCCC(=O)N1CCC(C(N)=O)(C(=O)NCC(=O)N2CCN(C(=O)[C@H](CCC(=O)NCCOCCOCCOCCOCCOCCOCCOCCOCCC(=O)N[C@@H](CCCCNC(=O)CCCc3ccc(Cl)cc3)C(=O)O)NC(=O)CN3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)CC1)(c1ccc(O)cc1)c1ccc(O)cc1]

Compound 23-Lu:

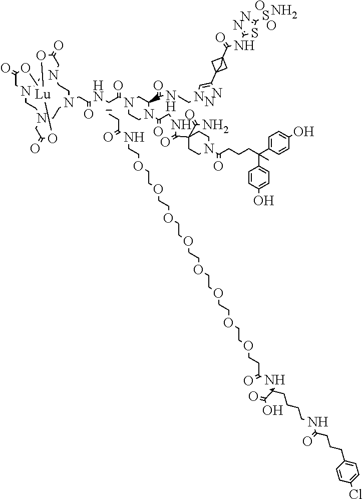

Compound 24:

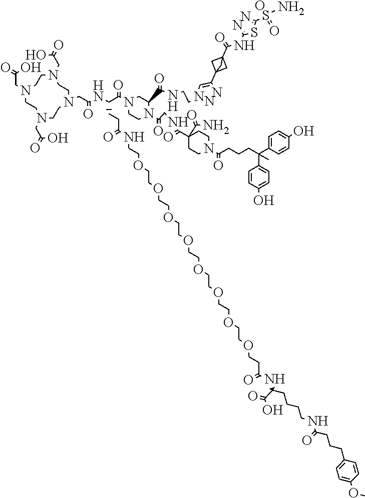 [SMILES: COc1ccc(CCCC(=O)NCCCC[C@H](NC(=O)CCOCCOCCOCCOCCOCCOCCOCCOCCNC(=O)CC[C@H](NC(=O)CN2CCN(CC(=O)O)CCN(CC(=O)O)CCN(CC(=O)O)CC2)C(=O)N2CCN(C(=O)CNC(=O)C3(C(N)=O)CCN(C(=O)CCCC(C)(c4ccc(O)cc4)c4ccc(O)cc4)CC3)[C@H](C(=O)NCCn3cc(C45CC(C(=O)Nc6nnc(S(N)(=O)=O)s6)(C4)C5)nn3)C2)C(=O)O)cc1]

Compound 24-Lu:

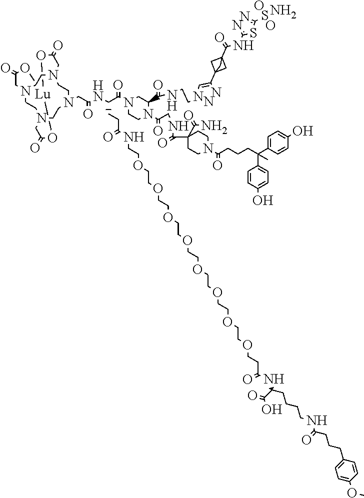

Compound 25:

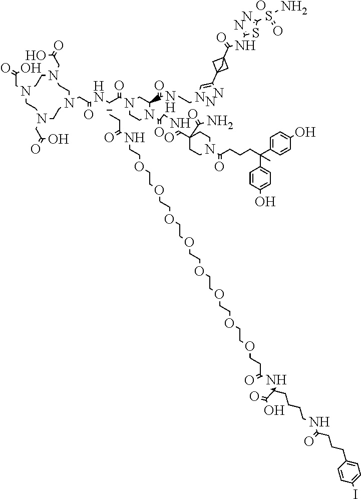 [SMILES: CC(CCCC(=O)N1CCC(C(N)=O)(C(=O)NCC(=O)N2CCN(C(=O)[C@H](CCC(=O)NCCOCCOCCOCCOCCOCCOCCOCCOCCC(=O)N[C@@H](CCCCNC(=O)CCCc3ccc(I)cc3)C(=O)O)NC(=O)CN3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)CC1)(c1ccc(O)cc1)c1ccc(O)cc1]

TABLE 3

| Compound | LCMS m/z | |
|---|---|---|
| 1 | 1144.4 | [M/2 + H]⁺ |
| 1-Cu | 1174.5 | [M/2 + H]⁺ |
| 2 | 1194.4 | [M/2 + H]⁺ |
| 2-Lu | 1280.8 | [M/2 + H]⁺ |
| 3 | 1148.4 | [M/2 + H]⁺ |
| 3-Pb | 1251.3 | [M/2 + H]⁺ |
| 4 | 1146.3 | [M/2 + H]⁺ |
| 4-Pb | 833.0 | [M/3 + H]⁺ |
| 5 | 1116.8 | [M/2 + H]⁺ |
| 5-Lu | 1202.8 | [M/2 + H]⁺ |
| 6 | 1140.8 | [M/2 + H]⁺ |
| 6-Lu | 1226.7 | [M/2 + H]⁺ |
| 7 | 1131.8 | [M/2 + H]⁺ |
| 7-Lu | 1217.8 | [M/2 + H]⁺ |
| 8 | 766.8 | [M/3 + H]⁺ |
| 8-Lu | 823.6 | [M/3 + H]⁺ |
| 8-In | 1204.5 | [M/2 + H]⁺ |
| 8-Tb | 1226.5 | [M/2 + H]⁺ |
| 9 | 750.5 | [M/3 + H]⁺ |
| 9-Lu | 807.6 | [M/3 + H]⁺ |
| 10 | 764.6 | [M/3 + H]⁺ |
| 10-Lu | 1233.5 | [M/2 + H]⁺ |
| 11 | 759.6 | [M/3 + H]⁺ |
| 11-Lu | 817.4 | [M/3 + H]⁺ |
| 12 | 748.9 | [M/3 + H]⁺ |
| 12-Lu | 806.8 | [M/3 + H]⁺ |
| 13 | 1115.0 | [M/2 + H]⁺ |
| 13-Lu | 801.2 | [M/3 + H]⁺ |
| 14 | 732.6 | [M/3 + H]⁺ |
| 14-Cu | 1129.2 | [M/2 + H]⁺ |
| 15 | 725.8 | [M/3 + H]⁺ |
| 15-Cu | 746.3 | [M/3 + H]⁺ |
| 16 | 731.1 | [M/3 + H]⁺ |
| 16-Cu | 751.7 | [M/3 + H]⁺ |
| 17 | 863.0 | [M/3 + H]⁺ |
| 17-Lu | 920.4 | [M/3 + H]⁺ |
| 18 | 1172.7 | [M/2 + H]⁺ |
| 18-Lu | 839.5 | [M/3 + H]⁺ |
| 19 | 741.5 | [M/3 + H]⁺ |
| 19-Lu | 799.0 | [M/3 + H]⁺ |
| 20 | 843.5 | [M/2 + H]⁺ |
| 20-Lu | 929.0 | [M/2 + H]⁺ |
| 21 | 833.4 | [M/2 + H]⁺ |
| 21-Lu | 919.4 | [M/2 + H]⁺ |
| 22 | 740.3 | [M/3 + H]⁺ |
| 22-Lu | 797.6 | [M/3 + H]⁺ |
| 23 | 1119.8 | [M/2 + H]⁺ |
| 23-Lu | 804.1 | [M/3 + H]⁺ |
| 24 | 745.3 | [M/3 + H]⁺ |
| 24-Lu | 802.6 | [M/3 + H]⁺ |
| 25 | 1165.4 | [M/2 + H]⁺ |
| 26 | 1147.3 | [M/2 + H]⁺ |
| 26-Pb | 1250.2 | [M/2 + H]⁺ |

### Example 5: Surface Plasmon Resonance Assay Conditions for hCA-IX and hCA-XII

Surface Plasmon Resonance (SPR) experiments were conducted on a Biacore 8K instrument (Cytiva, Marlborough, MA) to determine the binding kinetics of the compound to recombinant human CA-IX (Acro, CA9-H82E3) and CA-XII (Creative BioMart, CA12-261H) proteins. The ligands were diluted to 10 μg/mL in PBS-P+ buffer (20 mM phosphate buffer, pH 7.4, with 2.7 mM KCl, 137 mM NaCl, and 0.05% Tween 20) and captured on Series S Streptavidin Sensor Chips (Cytiva, 29699621) to 1200-1800 response units (RU) using a flow rate of 5 μL/min and injection times of 180 seconds for human CA-IX and 75 seconds for human CA-XII. Analyte binding was evaluated using a single-cycle kinetics method over 9 analyte concentrations (100-0.39 nM) in PBS-P+ buffer with 1% DMSO using a 50 μL/min. flow rate, 120 second association time, and 1200 second dissociation time. The flow cell temperature was set to 25° C. Analyte binding curves were fit to a 1:1 binding kinetic model in Biacore Insight Evaluation software (version 4.0.8.20368) following reference flow cell subtraction and DMSO correction to derive the association and dissociation binding constants. The data are presented in Table 4. This example demonstrates that the compounds described herein bind to CA IX with strong affinity. This example also demonstrates that the compounds described herein selectively bind to CA IX with stronger affinity than to CA XII.

TABLE 4

| Compound | CAIX KD (nM) | CAXII KD (nM) |
|---|---|---|
| 1-Cu | 0.0783 | 26.8 |
| 2-Lu | 0.0609 | 29.2 |
| 3-Pb | 0.0432 | 27.4 |
| 4-Pb | 0.0421 | 29.1 |
| 5-Lu | 0.0257 | 14.2 |
| 6-Lu | 0.0559 | 51.0 |
| 7-Lu | 0.0443 | 35.4 |
| 8-Lu | 0.0359 | 41.3 |
| 8-In | 0.133 | 47.2 |
| 8-Tb | 0.109 | 175 |
| 9-Lu | 0.0191 | 11.0 |
| 10-Lu | 0.0366 | 50.2 |
| 11-Lu | 0.0273 | 35.3 |
| 12-Lu | 0.0268 | 14.6 |
| 13-Lu | 0.0362 | 13.7 |
| 14-Cu | 0.0304 | 41.3 |
| 15-Cu | 0.0491 | 46.5 |
| 16-Cu | 0.0437 | 52.0 |
| 17-Lu | 0.295 | 60.1 |
| 18-Lu | 0.183 | 3730 |
| 19-Lu | 0.207 | 3120 |
| 20-Lu | 0.104 | 107.0 |
| 21-Lu | 0.0897 | 58.0 |
| 22-Lu | 0.203 | 197.0 |
| 23-Lu | 0.239 | 404.0 |
| 24-Lu | 0.184 | 101.0 |
| 25 | 0.169 | 221.0 |
| 26-Pb | 0.0501 | 36.2 |

A number of references have been cited, the disclosures of each of which are incorporated herein by reference in their entireties.

1-110. (canceled)

111. A compound having the following structure: 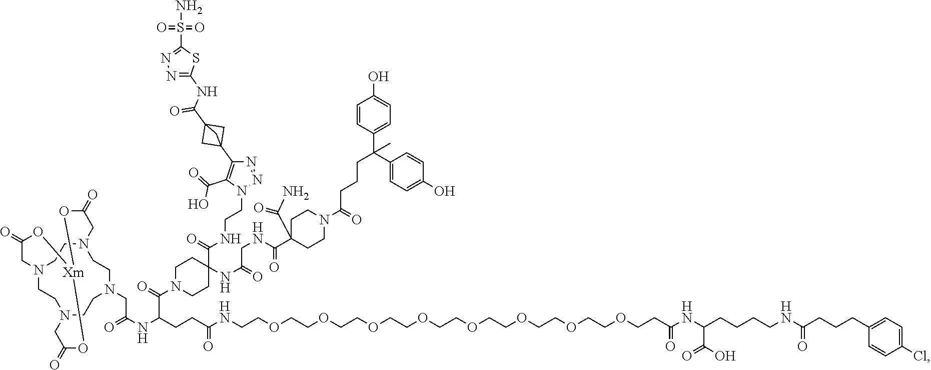
or a stereoisomer or mixture of stereoisomers or a pharmaceutically acceptable salt thereof, wherein X_{M} is an alpha particle-emitting radionuclide or a beta particle-emitting radionuclide.

112. The compound of claim 111, or a stereoisomer or mixture of stereoisomers or a pharmaceutically acceptable salt thereof, wherein X_{M} is an alpha particle-emitting radionuclide.

113. The compound of claim 112, or a stereoisomer or mixture of stereoisomers or a pharmaceutically acceptable salt thereof, wherein the alpha particle-emitting radionuclide is Ac-225, Bi-212, Bi-213, Bi-209, Tb-149, Ra-223, Ra-224, Th-227, Fr-223, Gd-148, Th-229, Pb-212, or Po-213.

114. The compound of claim 113, or a stereoisomer or mixture of stereoisomers or a pharmaceutically acceptable salt thereof, wherein the alpha particle-emitting radionuclide is Ac-225.

115. The compound of claim 111, or a stereoisomer or mixture of stereoisomers or a pharmaceutically acceptable salt thereof, wherein X_{M} is a beta particle-emitting radionuclide.

116. The compound of claim 115, or a stereoisomer or mixture of stereoisomers or a pharmaceutically acceptable salt thereof, wherein the beta particle-emitting radionuclide is Cu-67, Lu-177, Y-90, Rh-105, Yb-175, Tm-167, Pm-153, Sm-153, or Tb-161.

117. The compound of claim 116, or a stereoisomer or mixture of stereoisomers or a pharmaceutically acceptable salt thereof, wherein the beta particle-emitting radionuclide is Lu-177, Tb-161, or Cu-67.

118. The compound of claim 117, or a stereoisomer or mixture of stereoisomers or a pharmaceutically acceptable salt thereof, wherein the beta particle-emitting radionuclide is Lu-177.

119. The compound of claim 111, having the following structure: 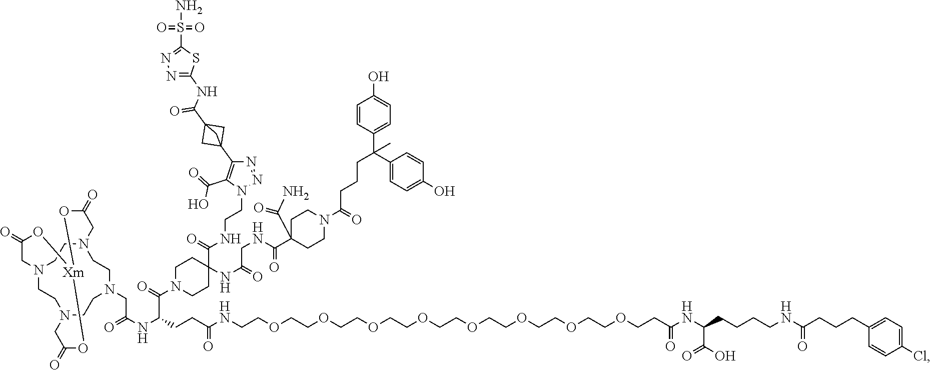
or a pharmaceutically acceptable salt thereof, wherein X_{M} is an alpha particle-emitting radionuclide or a beta particle-emitting radionuclide.

120. The compound of claim 119, or a pharmaceutically acceptable salt thereof, wherein X_{M} is an alpha particle-emitting radionuclide.

121. The compound of claim 120, or a pharmaceutically acceptable salt thereof, wherein the alpha particle-emitting radionuclide is Ac-225, Bi-212, Bi-213, Bi-209, Tb-149, Ra-223, Ra-224, Th-227, Fr-223, Gd-148, Th-229, Pb-212, or Po-213.

122. The compound of claim 121, or a pharmaceutically acceptable salt thereof, wherein the alpha particle-emitting radionuclide is Ac-225.

123. The compound of claim 119, or a pharmaceutically acceptable salt thereof, wherein X_{M} is a beta particle-emitting radionuclide.

124. The compound of claim 123, or a pharmaceutically acceptable salt thereof, wherein the beta particle-emitting radionuclide is Cu-67, Lu-177, Y-90, Rh-105, Yb-175, Tm-167, Pm-153, Sm-153, or Tb-161.

125. The compound of claim 124, or a pharmaceutically acceptable salt thereof, wherein the beta particle-emitting radionuclide is Lu-177, Tb-161, or Cu-67.

126. The compound of claim 125, or a pharmaceutically acceptable salt thereof, wherein the beta particle-emitting radionuclide is Lu-177.

127. A pharmaceutical composition comprising the compound of claim 111, or a stereoisomer or mixture of stereoisomers or a pharmaceutically acceptable salt thereof, and a pharmaceutically acceptable excipient or carrier.

128. A method of treating cancer in a subject in need thereof, the method comprising administering to the subject a therapeutically effective amount of the compound of claim 111, or a stereoisomer or mixture of stereoisomers or a pharmaceutically acceptable salt thereof.

129. A pharmaceutical composition comprising the compound of claim 119, or a pharmaceutically acceptable salt thereof, and a pharmaceutically acceptable excipient or carrier.

130. A method of treating cancer in a subject in need thereof, the method comprising administering to the subject a therapeutically effective amount of the compound of claim 119, or a pharmaceutically acceptable salt thereof.

131. A compound having the following structure: 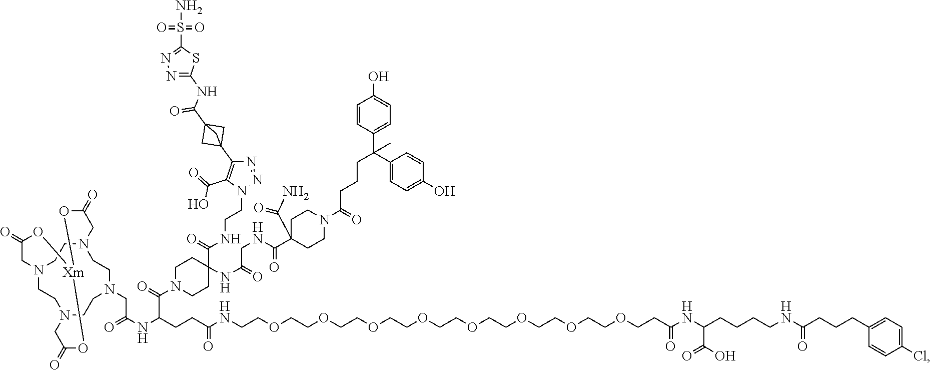
or a stereoisomer or mixture of stereoisomers or a pharmaceutically acceptable salt thereof, wherein X_{M} is a photon particle-emitting radionuclide.

132. The compound of claim 131, wherein the photon-emitting radionuclide is Tc-99m, In-111, Ga-67, Y-90, Lu-177, Sm-153, Re-186, Re-188, or Tb-161.

133. The compound of claim 132, or a stereoisomer or mixture of stereoisomers or a pharmaceutically acceptable salt thereof, wherein the photon-emitting radionuclide is In-111, Ga-67, Lu-177, or Tb-161.

134. The compound of claim 133, or a stereoisomer or mixture of stereoisomers or a pharmaceutically acceptable salt thereof, wherein the photon-emitting radionuclide is In-111 or Tb-161.

135. The compound of claim 134, or a stereoisomer or mixture of stereoisomers or a pharmaceutically acceptable salt thereof, wherein the photon-emitting radionuclide is Tb-161.

136. The compound of claim 134, or a stereoisomer or mixture of stereoisomers or a pharmaceutically acceptable salt thereof, wherein the photon-emitting radionuclide is In-111.

137. A pharmaceutical composition comprising the compound of claim 131, or a stereoisomer or mixture of stereoisomers or pharmaceutically acceptable salt thereof, and a pharmaceutically acceptable excipient or carrier.

138. A method of treating cancer in a subject in need thereof, the method comprising administering to the subject a therapeutically effective amount of the compound of claim 131, or a stereoisomer or mixture of stereoisomers or a pharmaceutically acceptable salt thereof.

139. The compound of claim 131, having the following structure: 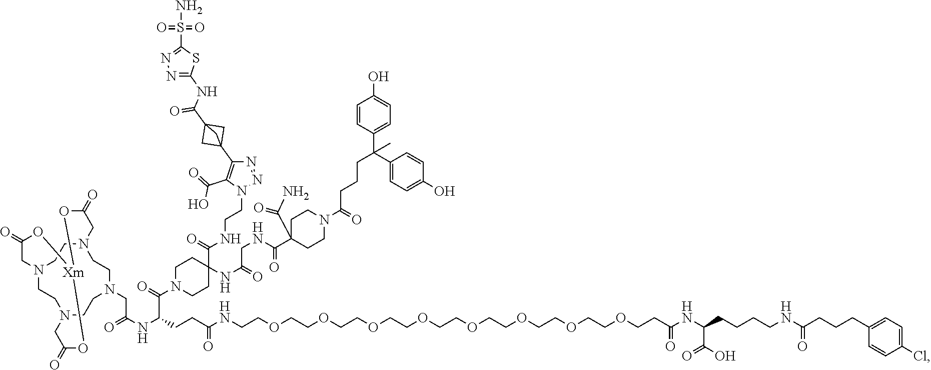
or a pharmaceutically acceptable salt thereof, wherein X_{M} is a photon particle-emitting radionuclide.

140. The compound of claim 139, or a pharmaceutically acceptable salt thereof, wherein the photon-emitting radionuclide is Tc-99m, In-111, Ga-67, Y-90, Lu-177, Sm-153, Re-186, Re-188, or Tb-161.

141. The compound of claim 140, or a pharmaceutically acceptable salt thereof, wherein the photon-emitting radionuclide is In-111, Ga-67, Lu-177, or Tb-161.

142. The compound of claim 141, or a pharmaceutically acceptable salt thereof, wherein the photon-emitting radionuclide is In-111 or Tb-161.

143. The compound of claim 142, or a pharmaceutically acceptable salt thereof, wherein the photon-emitting radionuclide is Tb-161.

144. The compound of claim 142, or a pharmaceutically acceptable salt thereof, wherein the photon-emitting radionuclide is In-111.

145. A pharmaceutical composition comprising the compound of claim 139, or a pharmaceutically acceptable salt thereof, and a pharmaceutically acceptable excipient or carrier.

146. A method of treating cancer in a subject in need thereof, the method comprising administering to the subject a therapeutically effective amount of the compound of claim 139, or a pharmaceutically acceptable salt thereof.

147. A compound of the following structural formula: 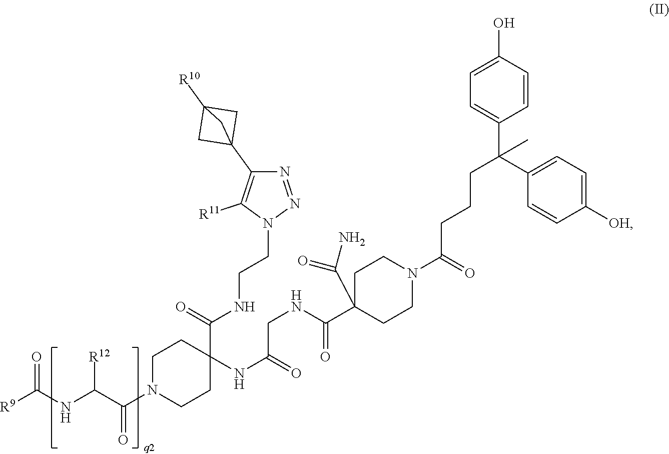
or a stereoisomer or mixture of stereoisomers, or a pharmaceutically acceptable salt thereof, wherein:
q2 is 1;
R¹² is 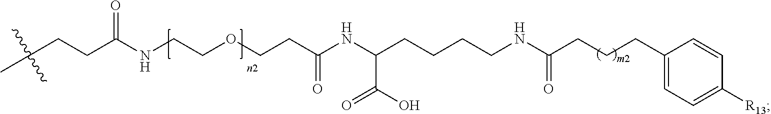
n2 is 2, 3, 4, 5, 6, 7, 8, 9, 10, 11, 12, 13, 14, 15, or 16;
m2 is 1, 2, or 3;
R¹³ is H, halogen, —CH₃, —OCH₃, or —OCH₂CH₃;
R¹¹ is H or —COOH;
R¹⁰ is 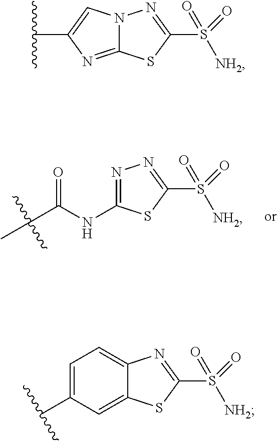 [SMILES: *c1cn2nc(S(N)(=O)=O)sc2n1 ; *C(=O)Nc1nnc(S(N)(=O)=O)s1 ; "or" ; *c1ccc2nc(S(N)(=O)=O)sc2c1]
and
R⁹ comprises a metal chelator (CL).
